(19) United States
(12) Patent Application Publication
(10) Pub. No.: US 2025/0263367 A1
(43) Pub. Date: Aug. 21, 2025

(54) A FACILE AND ODOR-FREE APPROACH TO CONVERT SULFONYL UREA DERIVATIVES TO CHALCOGENIDE SULFONYL UREA DERIVATIVES

(71) Applicant: THE UNITED STATES OF AMERICA, as represented by the Secretary, Department of Health and Human Serice, Rockville, MD (US)

(72) Inventor: Malliga Iyer, Rockville, MD (US)

(21) Appl. No.: 18/561,068

(22) PCT Filed: May 12, 2022
(86) PCT No.: PCT/US2022/028942

§ 371 Date: Nov. 15, 2023

Related U.S. Application Data
(60) Provisional application No. 63/189,375, filed on May 17, 2021.

(51) Int. Cl. C07C 315/04; C07D 231/06; C07D 401/12

(52) U.S. Cl. CPC C07C 315/04; C07D 231/06; C07D 401/12

(57) ABSTRACT

In one aspect, the application relates to sulfonyl chalcogenide urea derivatives of Formula II, including sulfonyl thioureas and sulfonyl selenoureas, and methods of making the same, wherein the methods can be carried out under mild conditions and do not require the use of malodorous reagents. In one aspect, the methods are one-pot, two-step reactions wherein an intermediate imidoyl chloride is treated with a compound having formula M₂X₂O₃ or M₂XJO₃ (where X and J can independently be Se, S, and/or Te) or a hydrate thereof, forming a Bunte salt which quickly decomposes to form the sulfonyl chalcogenide urea derivatives. The application also relates to substituted sulfonyl chalcogenide urea derivatives of Formula V and a method for their preparation from sulfonyl chalcogenide urea derivatives of Formula II, to a method for synthesizing a substituted guanidine from a sulfonyl chalcogenide urea of Formula II as well as to substituted guanidine derivatives thus prepared.

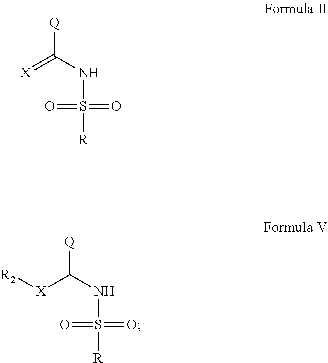

### CROSS REFERENCE TO RELATED APPLICATIONS

This application claims the benefit of U.S. Provisional Application No. 63/189,375 filed on May 17, 2021, which is incorporated herein by reference in its entirety.

### BACKGROUND

Chalcogenide elements of the periodic table, particularly sulfur in its various oxidation states, are proving to be indispensable in modern medicinal and environmental applications. Interrogating biological problems and treating complex diseases require new approaches to the generation of novel chemical probes and drug-like molecules often in scalable quantities.

Sulfonylureas (SUs) have long been used as antidiabetic agents and impact a range of biological pathways. They are reported to have anti-aging properties and proposed as auxiliary therapy for Alzheimer's disease. They have a role in ameliorating inflammatory pathways by acting as inflammasome inhibitors and are believed to inhibit interleukin-1 activity. SUs also act as potassium ATP channel modulators and acyl CoA: cholesterol O-acyl transferases (ACAT) inhibitors. (FIG. 1A). SUs are also used widely as herbicides, antihelminthics, and acaricides. Antimicrobial and fungicidal activity are also attributed to sulfonylurea compounds, while sulfonylthioureas (STUs) are purported to have insecticidal activity and anti-convulsant properties.

Many studies of sulfonyl ureas test a limited number of these analogs, possibly due to the intractable routes to access the corresponding chalcogenide ureas. While sulfonyl thioureas can be expected to have similar valuable properties to their oxygen counterparts, little is known about the sulfonyl selenoureas. One important utility of thiourea analogs has been demonstrated in the synthesis of therapeutically important guanidine compounds in the presence of HgCl₂. Generation of Se/S-ureas would also enable the synthesis of iso-“chalcogenide”-urea derivatives, which may have novel properties and applications.

Current methods to convert urea to thiourea containing derivatives involve use of Lawesson's reagents, P₂S₅, thiourea, NaHS, H₂S, and other toxic and malodourous reagents under higher reflux temperatures. Woollins reagent has been used to convert ureas to selenoureas. Such conversions, however, are not suitable for obtaining sulfonyl thioureas or selenoureas from sulfonyl ureas as the reactions do not proceed or proceed in low isolated yields. A commonly used process for synthesis of thioureas involve unstable isothiocyanates (FIGS. 1A-1F).

Organic thiosulfates (RSSO₃M, M=Na, K)) are commonly known as Bunte salts and are known to be used as “sulfur surrogates.” Alkali metal S-alkylthiosulfates and S-aryl thiosulfate are known as Bunte salts after Hans Bunte, who first reported sodium S-ethyl thiosulfates. When sodium S-ethyl thiosulfate is hydrolyzed in the presence of an acid, only ethyl thiol is formed. Similarly, alkyl selenosulfates or “seleno Bunte salts” have been prepared where one selenium atom replaces one sulfur atom in Bunte salts.

A direct synthesis of thiols from halo-heterocycles using Na₂S₂O₃ has been reported. Recently it was shown that 2-chloro-4,5-dihydroimidazole hemisulfate was subjected to a reaction with a 2-fold excess of sodium thiosulfate in aqueous solution at room temperature. An exothermic nucleophilic substitution reaction has also been reported, followed by a vigorous evolution of sulfur trioxide possibly forming the internal Bunte salt which upon immediate decomposition formed imidazolidine-2-thione in quantitative yields.

Many applications explore a limited number of sulfonylthioureas (STUs), possibly due to the lack of robust routes to generate them. Thiourea analogs have important utility in the synthesis of therapeutically important guanidine compounds in the presence of HgCl₂. Generation of STUs would also enable the synthesis of isothiourea derivatives which can have novel properties and applications.

Despite advances in synthesis of sulfonyl ureas and chalcogenide ureas, there is still a scarcity of methods using mild conditions and non-malodorous reagents that provide high yields with little decomposition of products and/or important synthetic intermediates. There is additionally a scarcity of robust routes for synthesizing sulfonylthioureas. Ideally, such methods would be compatible with alkylation of the resulting compounds, further extending the potential applications of the methods. It would additionally be desirable to generate novel, non-traditional chemotypes with chalcogenide-bearing stereocenters, which are one of the underexplored pharmacophores in the biological realm (FIGS. 1B-1C). These needs and other needs are satisfied by the present disclosure.

### SUMMARY

In accordance with the purpose(s) of the present disclosure, as embodied and broadly described herein, the disclosure, in one aspect, relates to sulfonyl chalcogenide ureas, including sulfonyl thioureas and sulfonyl selenoureas, and methods of making the same, wherein the methods can be carried out under mild conditions and do not require the use of malodorous reagents. In one aspect, the methods are one-pot, two-step reactions wherein an intermediate imidoyl chloride is treated with a compound having formula M₂X₂O₃ or M₂XJO₃ (where X and J can independently be Se, S, and/or Te) or a hydrate thereof, forming a Bunte salt which quickly decomposes to form the disclosed sulfonyl chalcogenide ureas. Also provided herein are alkylated sulfonyl chalcogenide ureas and methods for making the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F show objectives and obstacles for the disclosed chalcogenide sulfonylurea augmentation strategy. FIG. 1A: Sulfonylurea-derived (SU) motif in biologically active molecules. FIG. 1B: Underexplored chalcogenide sulfonyl entities. FIG. 1C: Current obstacles in the chalcogenide augmentation process. FIG. 1D: General approaches for thiourea synthesis. FIG. 1E: Sulfur surrogate/Bunte Salt approach for simple thiourea synthesis. FIG. 1F: A general strategy for chalcogenide augmentation from SU: present work.

FIGS. 2A-2E show synthesis of sulfonylthioureas (STU) from SU including optimization of reaction conditions FIG. 2A: Streamlined process for sulfonylthiourea synthesis FIG. 2B: Optimization of Conditions for thiosulfate displacement. FIG. 2C: LCMS chromatogram of reaction products (Conditions #3) FIG. 2D: Synthesis of “Thio” chlorpropamide FIG. 2E: Synthesis of “Thio” tolbutamide.

FIG. 3 shows diversity and functional group compatibility for the SU to STU conversion.

FIGS. 4A-4B show application and modifications for diversity-oriented synthesis. FIG. 4A: Building block diversity in 3-step, one-pot sequential augmentation based on sulfur. FIG. 4B: Entry into seleno-augmentation of sulfonylurea products.

FIG. 5 shows a crystal structure for compound 3m disclosed herein.

FIG. 6 shows a crystal structure for compound 31 13 disclosed herein.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a reaction of the sulfonyl ureas with a source of chloride to give intermediate imidoyl chlorides which, upon treatment with Bunte agents, forming Bunte intermediates that decompose to the sulfonyl chalcogenide ureas, including sulfonyl thioureas and sulfonyl selenoureas in excellent yields. In one aspect, this one-pot, two-step protocol is a vast improvement over previous attempts to generate sulfonyl thioureas, such as from refluxing xylenes in the presence of P₂S₅ and under microwave conditions. In a further aspect, this methodology is expected to have wide applicability in generation of thiourea and isothiourea analogs.

Simple SUs include FDA approved agents like chlorpropamide and tolbutamide, which are commercially available and could support feasibility studies. 3,4-diarylpyrazoline sulfonylureas and thioureas are purported to have insecticidal activity. They also serve as precursors to potent cannabinoid-1 (CB1) receptor blockers which are currently being explored for ameliorating a range of health conditions. Initially, the generation of 3,4-diarylpyrazoline sulfonylthioureas directly from the sulfonylurea intermediate precursors was explored.

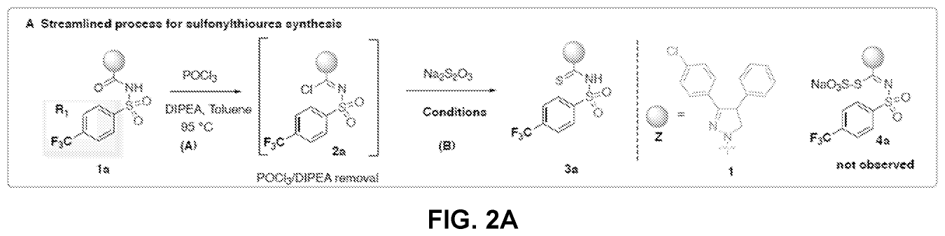

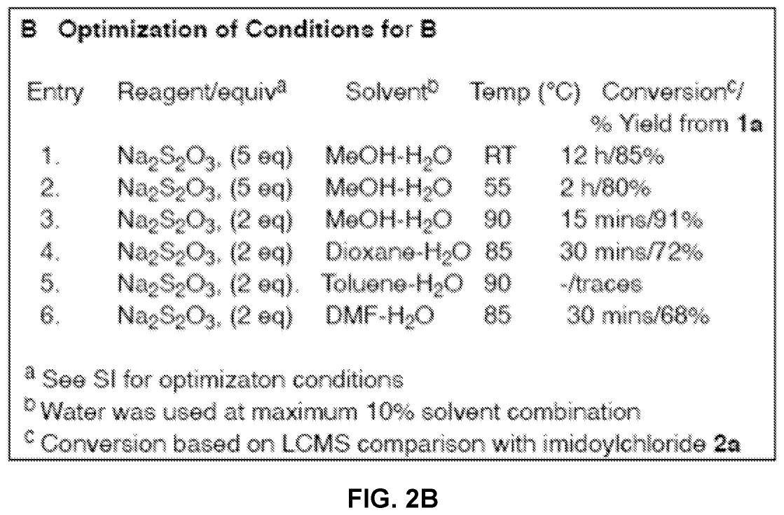

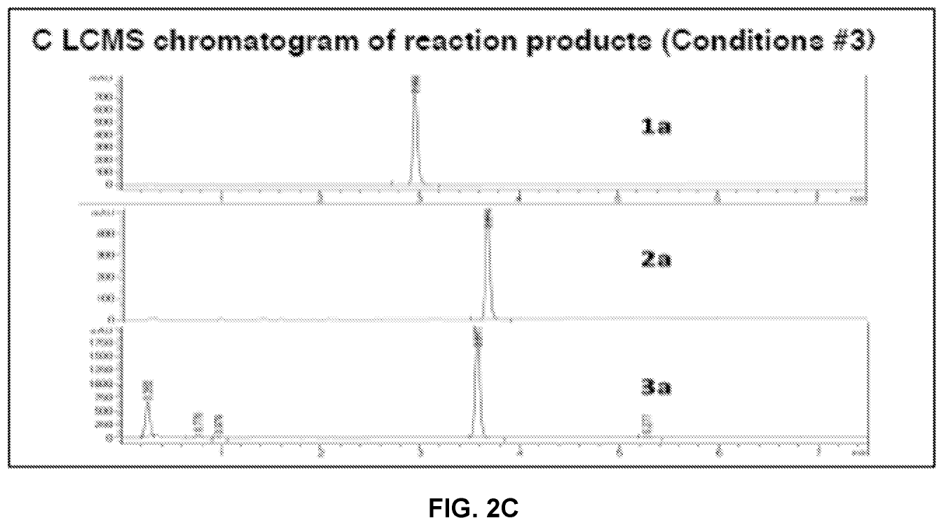

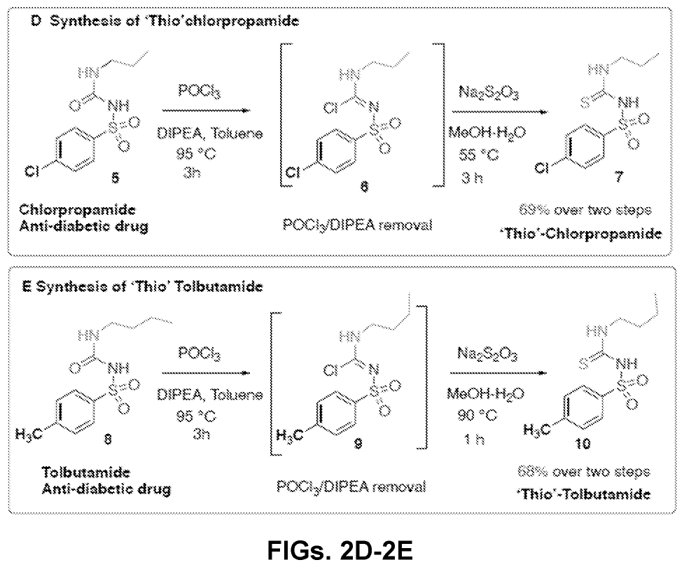

Additionally, a robust way of generating sulfonylthioureas would help practicing chemists to assay the properties of SU and STU in parallel. In an optimized approach, herein is shown the successful execution of the reaction depicted in FIGS. 2A-2E. Disclosed herein is a protocol where an inexpensive, odor free and safe, inorganic thiosulfate can displace a SU imidoylchloride to deliver clean, isolable sulfonyl containing thioureas. Using 1a as the model system, imidoylchloride 2a could be obtained cleanly from substrate 1a by treatment with POCl₃/DIPEA at 95° C. Upon subsequent treatment with Na₂S₂O₃ ($0.05/g) the putative formation of a Bunte intermediate (not detected) led to the generation of carbothioamide analogs in excellent yield (FIGS. 2A-2B). This one-pot, two-step protocol with or without the isolation of the intermediate imidoylchloride bypasses the need for sensitive isothiocyanate precursors. The imidoylchloride intermediate could also be generated from PCl₅ in refluxing chlorobenzene although decomposition side products were also seen. The nucleophilic displacement of imidoylchloride 2a by the thiosulfate ion worked best in methanol/H₂O at 90° C. and the thiourea analog could be generated within 20 mins. The nucleophilic displacement also worked well in methanol/H₂O at 55° C. with excess (5 eq) Na₂S₂O₃ over a period of 2 h. The reaction worked sluggishly at room temperature and proceeded to give 3a over extended reaction times (˜12 h). DMF/H₂O, ethanol/H₂O, acetonitrile-water were acceptable solvents for the reaction. The reaction also proceeded in aqueous dioxane although somewhat slower, and only traces of product could be obtained in aqueous toluene. Water was deemed essential for solubilization of the inorganic reactant, albeit the reaction did proceed in alcoholic solvents likely due to adventitious water. In general, it was observed that with substrate 1a, the reaction had a large range of temperature flexibility where the STU product 3a could be obtained without significant loss of yield.

With the optimized protocol in hand, it was tested whether the antidiabetic drugs chlorpropamide and tolbutamide would undergo the oxo-edits. Indeed, on a gram-scale, both compounds underwent the imidolylchloride conversion and the subsequent thiosulfate displacement leading to clean corresponding thiourea products in yields greater than 65%.

### Reaction Scope

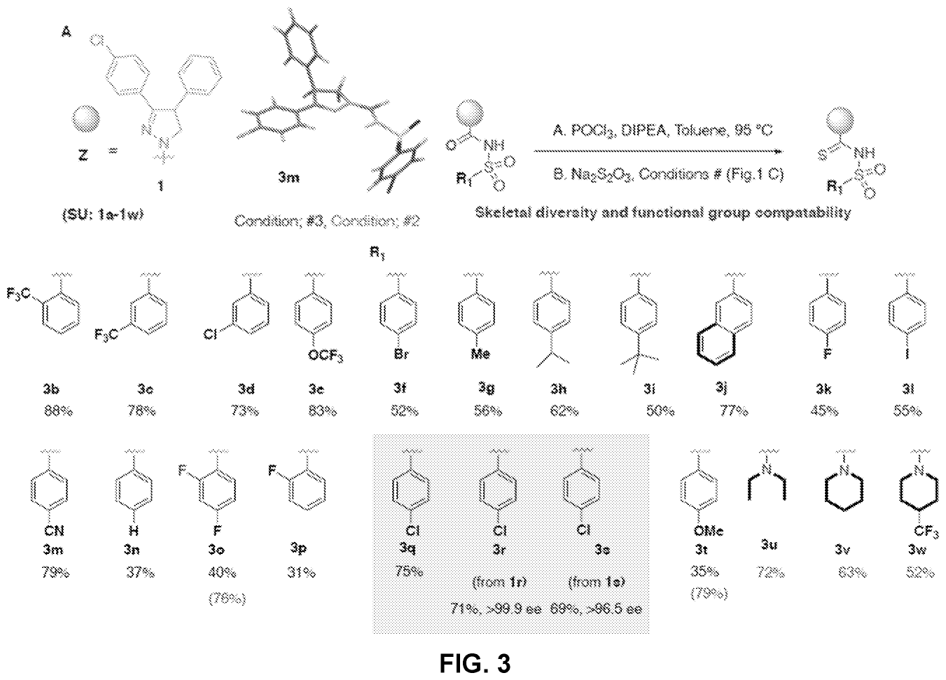

Having ready access to a range of 3,4-diarylpyrazoline SU products, the scope of conversion of 3,4-dihydropyrazoline sulfonylureas precursors to substituents that could be tolerated on the arylsulfonyl part of the molecule was tested. Many of the commonly used groups for structure-activity relationship studies (SAR) in biological applications (1a-1t) were very well tolerated under the optimized reaction conditions. As seen in FIG. 3, a wide variety of substituted sulfonylurea precursors40 proceeded to provide the STU in good to excellent yields.

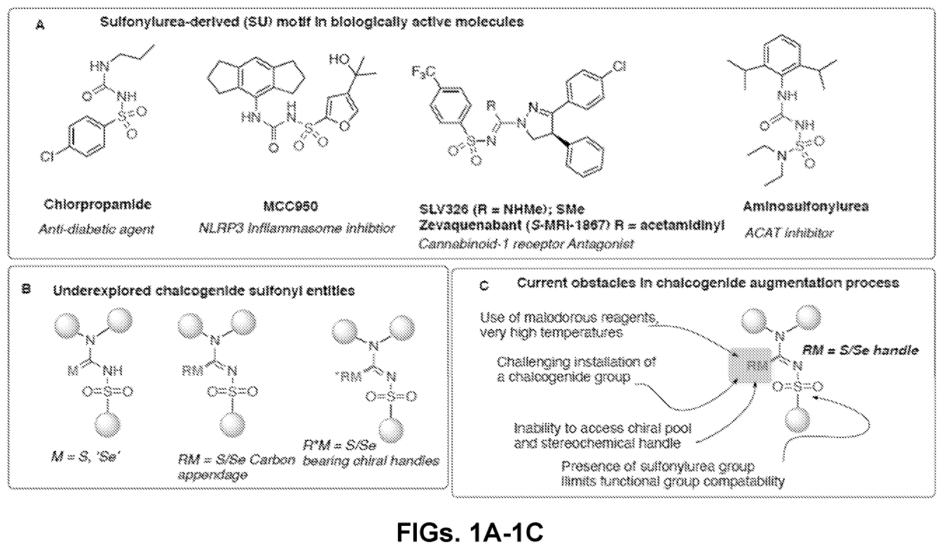

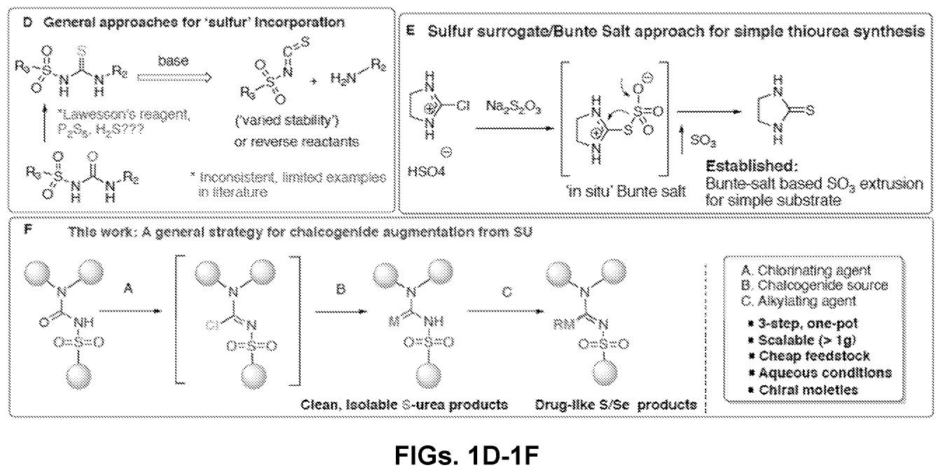

3,4-Dihydropyrazoline compounds of the type 1/3 bear a stereocenter at the C4 pyrazoline ring. Therefore, in case of example 1q (4-chlorophenyl substituent) the racemic, R as well as S enantiomer of the SU precursor were used to generate the corresponding thiourea products. Gratifyingly, it was seen that the two-step, one-pot protocol proceeded to give the thio-products with no erosion of chirality as documented by chiral HPLC. Replacement of the aryl groups at the sulfonyl end with dialkyl or cyclic amino pendants in the 3,4-diarylpyrazoline SU precursors (1u-1w) also provided the sulfonylthioureas (3u-3w) in yields above 50%. Conditions 2 in FIG. 1B were deemed to be optimal for substrates 1u-1w. It was also observed that yields of the products were largely unaffected by the electronics on the sulfonyl end of the molecule. The scope of the substituents at the non-sulfonyl end of the molecule was then tested.

Various amino precursors could be utilized at this end of the molecule. Replacement of the pyrazoline core with various attachments (e.g., pyridazinyl, cyclopentyl, cyclohexyl, and the like) also led to the conversion of urea to thiourea sulfonyl moieties. Subtle differences in temperature to affect the thiosulfate displacement and use of aqueous dioxane in lieu of methanol was required to obtain the thiourea products in such cases.

### Modification Approaches

To maximize the utility of this new protocol, it was posited that the Bunte-salts can be alkylated ‘in situ’ to generate sulfonylisothiourea analogs. Treatment of 3a reaction mixture containing the preformed ‘masked STU’ underwent facile alkylation in the same pot upon reaction with methyl iodide, resulting in a clean conversion to an S-methylated analog. It should be noted that the reaction required a slight excess of the alkylating precursor for complete transformation. A clear hint that the reaction occurs on the putative Bunte-salt 4 with concomitant alkylation along with SO₃ extrusion was seen with faster reaction times of the ‘in-situ’ alkylation as opposed to the alkylation in methanol after thiourea isolation.

That this reaction could proceed smoothly under wet alcoholic conditions gave us hope that other alkylation agents/STU may also be amenable to this transform, yielding novel isothiosulfonyl molecular scaffolds. Importantly, unprotected alkylating precursors like butynyl bromide (18/23) bromo-acetic acid and bromoethanol were used to yield chemotypes 22 and 25 respectively on different STU scaffolds which could be primed for downstream coupling manipulations. It became clear to us that chiral alkylating agents could also be used to drive this synthesis towards stereoselective products. Expanding on those lines, the strategy was successfully executed by the reaction of racemic 1a with methyl(S)-3-bromo-2-methylpropanoate to uneventfully yield 19 as a diastereomeric mixture. Similarly, racemic 1q could be turned into a diastereomeric mixture 20 using a chiral alkylating agent ((S)-1-bromo-2-methylbutane).

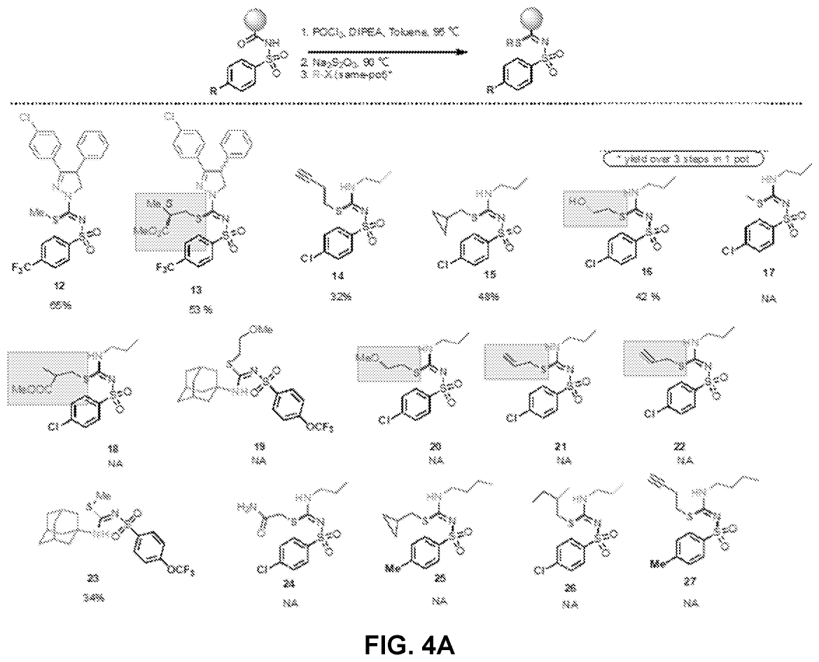

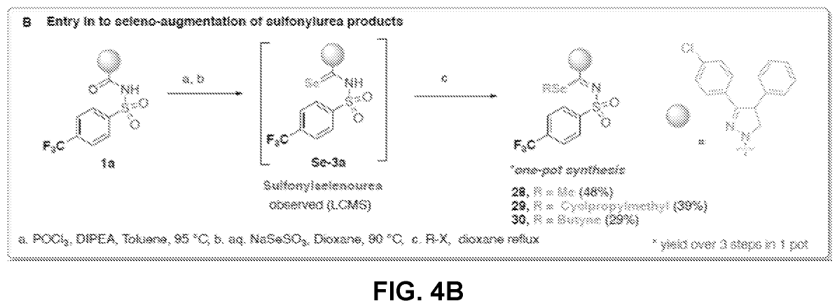

We also briefly evaluated the utility of this process in accessing isoselenosulfonyl urea analogs where the sulfonylurea oxygen is replaced by a selenium appendage. In examples shown in FIG. 4B, preformed NaSeSO₃ treatment of the imidoylchloride followed by an alkyl halide quench afforded the novel seleno analogs. The selenosulfonyl urea compound was too labile to allow clean isolation, but the existence of selenosulfonyl urea (and selenosulfonyl Bunte salt) could be confirmed by its prompt ‘in situ’ trapping to afford selenoalkylated analogs. Dioxane was preferred as the solvent over methanol to afford a facile conversion. Thus, this protocol would open avenues for the generation of novel seleno-mimics and warheads. With the myriad of combinations available as building blocks for amino, sulfonyl and alkylating agents one could envision constructing a diverse chalcogenide screening library based on this synthetic platform.

Finally, this methodology can be exploited in the preparation of substituted 4,5-dihydro-1H-pyrazoles/pyridazolines that are useful as potent cannabinoid receptor antagonists. Recently, such scaffolds have been employed to generate novel, selective agents that have potential in treating fibrosis as well as obesity and related metabolic disorders. Thus, there remains a need for an efficient, high-yielding, and scalable synthetic approach to provide substantial amounts of enantiomerically pure active pharmaceutical ingredients (APIs) in the chiral pyrazoline series for biological evaluation. The newly developed protocol was applied by utilizing an isothiourea-based chiral auxiliary/decoy intermediate to generate diastereomeric isothiourea precursors, which could be displaced by amino pendants to give a chiral supercritical chromatography (SFC)-free approach to generate enantiomeric APIs.

Compounds of the type 1a have a singular quaternary carbon stereocenter at the C4 position of the pyrazoline ring. Previous methods have used chiral HPLC/SFC separation of the final racemic mixture to yield the biologically active S-enantiomer. Since the disclosed methodology was amenable to multi-gram scale synthesis of STU precursor 3a (71% yield on a standalone 2.0 g scale), the precursor of type 13 (via 19) was synthesized through an in situ chiral alkylating agent bearing a known stereocenter. This ensured access to a separable diastereomeric mixture which could afford a single diastereomer of the type 13. Displacement of the thioalkyl group under controlled basic conditions can then deliver the requisite enantiomeric API. The diastereomeric mixture 19 thus enabled the generation of single diastereomer 13 through a preferential recrystallization. Chiral HPLC analysis showed that the crystals were indeed enriched in one of the diastereomeric forms (S, S). The stereochemistry of the diastereomer was assigned by separation on R,R-Whelk-O chiral column and X-ray crystallography. Knowing the stereochemistry at the S-alkylation center, the S,S-diastereomer was treated individually with methyl amine in a DCM:MeOH/Et₃N mixture to obtain SLV326. Compounds including but not limited to Ibipinabant, JD5047, Zevaquenabant, and other similar pyrazoline/pyridazinyl attachment-containing compounds can also be synthesized using this decoy intermediate approach.

### Definitions

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems.

Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃. The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, (haloR^{●}), —(CH₂)₀₋₂OH, —(CH₂)₀₋₂OR^{●}, —(CH₂)₀₋₂CH(OR)₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂)₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR), —OH, —OR*, —O(haloR*), —CN, —C(O)OH, —C(O)OR*, —NH₂, —NHR′, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†}) S(O)₂R^{†}; wherein each R^{†} is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR′, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

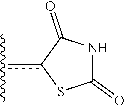

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms, and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

A “Bunte agent” as used herein refers to a salt useful for reacting with imidoyl chlorides which, upon treatment with the Bunte agents, forming Bunte intermediates that decompose to the sulfonyl chalcogenide ureas. In one aspect, a Bunte agent can have the formula M₂X₂O₃·mH₂O or M₂XJO₃·mH₂O, wherein M can be an alkali metal (e.g., Li, Na, K) and wherein X and J can independently be the same or a different chalcogenide (e.g., S, Se, Te). The Bunte agent can be a hydrate (e.g. where m is from 1 to 6) or can be anhydrous (e.g. where m is 0). In one aspect, Bunte agents useful herein include Na₂S₂O₃·5H₂O, Na₂S₂O₃, K₂S₂O₃, K₂Se₂O₃, Na₂SeSO3₃, related compounds, and combinations thereof.

Compounds described herein can contain one or more double bonds and thus, potentially give rise to cis/trans (E/Z) isomers, as well as other conformational isomers. Unless stated to the contrary, the invention includes all such possible isomers and tautomers (e.g., thiol/thiourea), as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or(S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, and chlorine, such as ²H, ³H, ¹³C, ¹⁴C, ¹⁵N, ¹⁸O, ¹⁷O, ³⁵S, ⁷⁴Se, ⁷⁶Se, ⁷⁷Se, ⁷⁸Se, ⁸⁰Se, ¹²²Te, ¹²³Te, ¹²⁴Te, ¹²⁵Te, ¹²⁶Te, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-¹⁴, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

It is also appreciated that certain compounds described herein can be present as an equilibrium of tautomers. For example, ketones with an α-hydrogen can exist in an equilibrium of the keto form and the enol form.

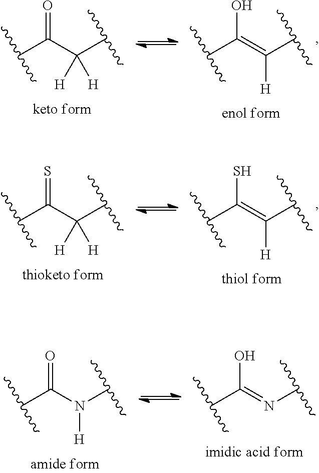 [SMILES: *CC(*)=O ; */C=C(/*)O ; *CC(*)=S ; */C=C(/*)S ; *NC(*)=O ; */N=C(/*)O]

Likewise, amides with an N-hydrogen can exist in an equilibrium of the amide form and the imidic acid form, and thiols can exist in a thiol/thioketo equilibrium, and the like. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

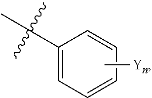

which is understood to be equivalent to a formula:

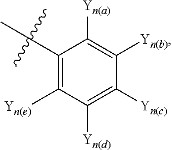

wherein n is typically an integer. That is, Yₙ is understood to represent five independent substituents, Yₙ₍ₐ₎, Y_{n(b)}, Y_{n(c)}, Y_{n(d)}, and Yₙ₍ₑ₎. By “independent substituents,” it is meant that each Y substituent can be independently defined. For example, if in one instance Yₙ₍ₐ₎ is halogen, then Y_{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a Bunte agent,” “a solvent,” or “a source of chloride,” includes, but is not limited to, mixtures or combinations of two or more such Bunte agents, solvents, or sources of chloride, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Methods for Synthesizing Sulfonyl Chalcogenide Ureas

Utilization of Lawesson's reagent in lieu of P₂S₅ resulted in inconsistent yields and in many cases the reaction proceeding to complete decomposition. A modified approach of the protocol using P₂S₅ saw some improvement under microwave conditions at 170° C. in xylenes. However, this protocol required the use of excess precautionary measures to contain the stench of sulfur reagents. Furthermore, the application of Woollins reagent in similar conditions did not yield the selenourea. Additional attempts using LiAlHSH or LiAlHSeH (Ishihara reagent) were unsuccessful.

Since 3,4-diaryl pyrazoline based scaffolds have numerous potential applications in the pharmaceutical and other industries, generating 3,4-diarylpyrazoline sulfonyl thioureas and selenoureas directly from sulfonyl urea intermediate precursors is desirable. Disclosed herein is a synthesis of sulfonyl thioureas and sulfonyl selenoureas from sulfonyl ureas under refluxing toluene in the presence of a chloride source.

In one aspect, disclosed herein is a method for synthesizing a sulfonyl chalcogenide urea, the method including at least the following steps:
(a) admixing a sulfonyl urea with a source of a leaving group to produce a first composition; and
(b) admixing the first composition with a compound having the formula M₂X₂O₃·mH₂O or M₂XJO₃·mH₂O to produce the sulfonyl chalcogenide urea;
wherein M is selected from K, Na, or Li;
wherein X and J are independently selected from S, Se, or Te; and
wherein m is from 0 to 6.

In one aspect, the leaving group can be a halide or another leaving group such as, for example, tosyl, triflyl, mesyl, or the like. In another aspect, the halide can be chloride, bromide, or iodide.

In another aspect, disclosed herein is a method for synthesizing a sulfonyl chalcogenide urea, the method including at least the following steps:
(a) admixing a compound of Formula I with a source of a leaving group to form a first composition:

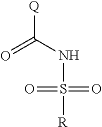

and
(b) admixing the first composition with the compound having the formula M₂X₂O₃·mH₂O or M₂XJO₃·mH₂O to form the sulfonyl chalcogenide urea having Formula II:

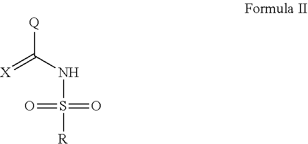

wherein X and J are independently selected from sulfur, selenium, or tellurium;
wherein Q is selected from substituted or unsubstituted amine, substituted or unsubstituted heterocycloalkyl group, or a substituted or unsubstituted heteroaryl group, or any combination thereof; and
wherein R is selected from a substituted or unsubstituted amine, a substituted or unsubstituted alkyl group, cycloalkyl group, heterocycloalkyl group, or a substituted or unsubstituted aromatic group, or any combination thereof.

In one aspect, the source of leaving group can be a halogenating agent. In another aspect, many halogenating agents are known in the art and can be used in the practice of the disclosed methods. In another aspect, these include, but are not limited to, dialkyl and/or diaryl chloroiminium ion compounds, phosgene, oxalyl dichloride, thionyl chloride, phosphorus pentachloride, phosphorous trichloride, phosphorus oxychloride, carbonyl dibromide, oxalyl bromide, thionyl bromide, phosphorous bromide, and phosphorus oxybromide. In another aspect, halogenating agents can be used alone or in combination.

In one aspect, the halogenating agent can be a phosphorus halide having the formula PW₃, POW₃, PW₅, and wherein W is selected from Cl, Br, or I. In one aspect, the source of leaving group can be a phosphorus chloride compound such as, for example, PCl₅ or POCl₃. In some aspects, step (a) further includes admixing a base with the compound of Formula I and the source of a leaving group. In one aspect, the base can be N,N-diisopropylethylamine (DIPEA), triethylamine, imidazole, benzimidazole, guanidine, ammonium hydroxide, pyridine, sodium hydroxide, potassium hydroxide, 4-dimethylaminopyridine (DMAP), 1,8-diazabicyclo[5.4.0]undec-7-ene (DBU), or any combination thereof.

In any of these aspects, step (a) can be carried out in a solvent such as, for example, toluene, chlorobenzene, dimethylformamide, dichloromethane, xylenes, or any combination thereof.

In an aspect, the compound having the formula M₂X₂O₃·mH₂O can be Na₂S₂O₃19 5H₂O, Na₂S₂O₃, K₂S₂O₃, K₂Se₂O₃, or Na₂SeSO₃. In one aspect, X can be sulfur and the compound having the formula M₂X₂O₃·mH₂O can be Na₂S₂O₃ or K₂S₂O₃. In another aspect, X can be selenium and the compound having the formula M₂X₂O₃·mH₂O or M₂XJO₃·mH₂O can be K₂Se₂O₃ or Na₂SeSO₃.

In some aspects, step (b) can further include admixing an additive with the compound of Formula II and the compound having the formula M₂X₂O₃·mH₂O or M₂XJO₃·mH₂O. In one aspect, the additive can be an organic or inorganic base including, but not limited to, triethylamine, Li₂CO₃, Na₂CO₃, K₂CO₃, MgCO₃, CaCO₃, BaCO₃, LiHCO₃, NaHCO₃, KHCO₃, Mg(HCO₃)₂, Ca(HCO₃)₂, tetrabutylammonium bromide, tetramethylammonium chloride, or any combination thereof. In another aspect, step (b) can be carried out in a solvent such as, for example, methanol, water, ethanol, dioxane, tetrahydrofuran, dimethylformamide, acetone, acetonitrile, DMSO, toluene, isopropyl alcohol, xylenes, ethylene glycol, or any combination thereof.

In any of these aspects, the compound having the formula M₂X₂O₃·mH₂O or M₂XJO₃·mH₂O, the sulfonyl chalcogenide urea, and/or any other reactant or product are not malodorous. In one aspect, either or both steps of the method can be carried out at a temperature of from about room temperature to about 140° C., or at about 20, 25, 30, 35, 40, 45, 50, 55, 60, 65, 70, 75, 80, 85, 90, 95, 100, 105, 110, 115, 120, 125, 130, 135, or about 140° C.

In one aspect, Q can be selected from

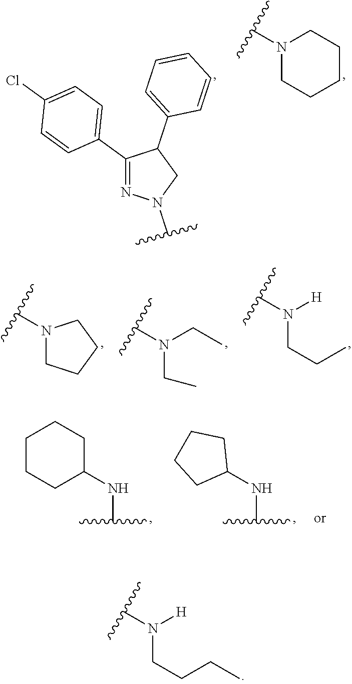

In another aspect, R can be selected from

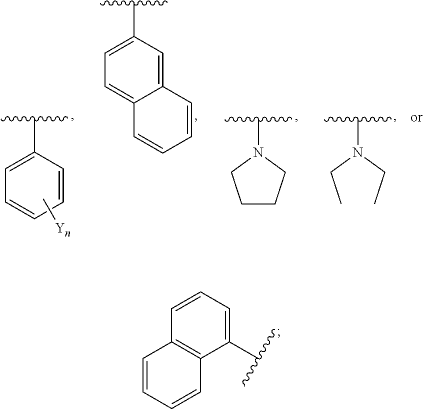

wherein each Y is independently selected from hydrogen, substituted or unsubstituted linear or branched C1-C10 alkyl, nitro, substituted or unsubstituted amino, halogen, cyano, alkoxy, thiol, phosphonate, haloalkyl, or haloalkoxy;
and wherein n is an integer from 1 to 5.

In some aspects, prior to step (a), a sulfonyl urea starting material can be produced in situ by reacting a sulfonyl carbamate or an isocyanate with a substituted or unsubstituted amine.

In some aspects, the compound of Formula I can be formed in situ by the reaction of a an isocyanate or a compound of Formula III or IIIa and a compound having formula Q-H:

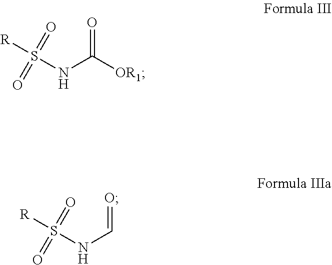

wherein R₁, if present, can be a substituted or unsubstituted linear or branched alkyl, aryl, or aralkyl group; and
wherein R is selected from a substituted or unsubstituted amine, a substituted or unsubstituted alkyl group, cycloalkyl group, heterocycloalkyl group, or a substituted or unsubstituted aromatic group, or any combination thereof.

In one aspect, R₁ can be methyl, tert-butyl, or phenyl.

Also disclosed are sulfonyl chalcogenide ureas produced by the disclosed methods.

### Sulfonyl Chalcogenide Ureas

In one aspect, disclosed herein are sulfonyl chalcogenide ureas having Formula II:

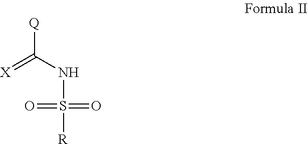

wherein X is sulfur, selenium, or tellurium;
wherein Q is selected from substituted or unsubstituted amine, substituted or unsubstituted heterocycloalkyl group, or a substituted or unsubstituted heteroaryl group, or any combination thereof; and
wherein R is selected from a substituted or unsubstituted amine, a substituted or unsubstituted alkyl group, cycloalkyl group, heterocycloalkyl group, or a substituted or unsubstituted aromatic group, or any combination thereof.

In one aspect, Q, R, and X are as defined above.

In still another aspect, the compound of Formula II can be selected from

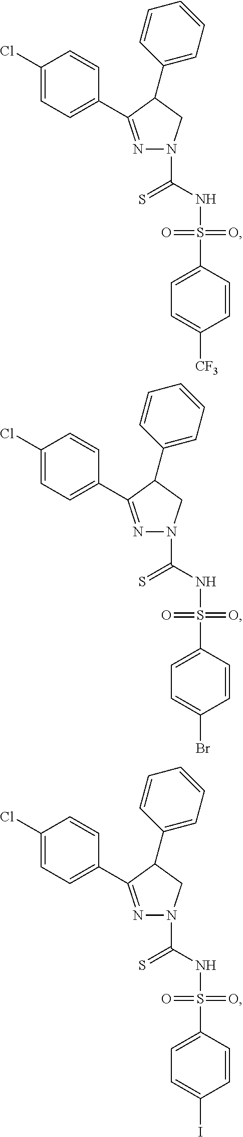

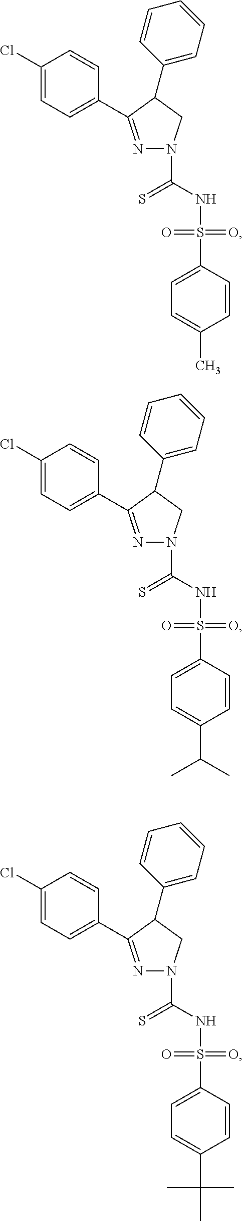

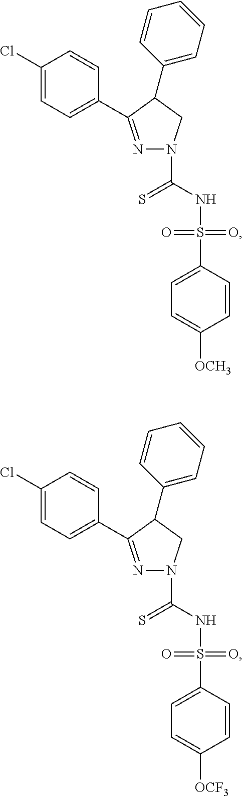

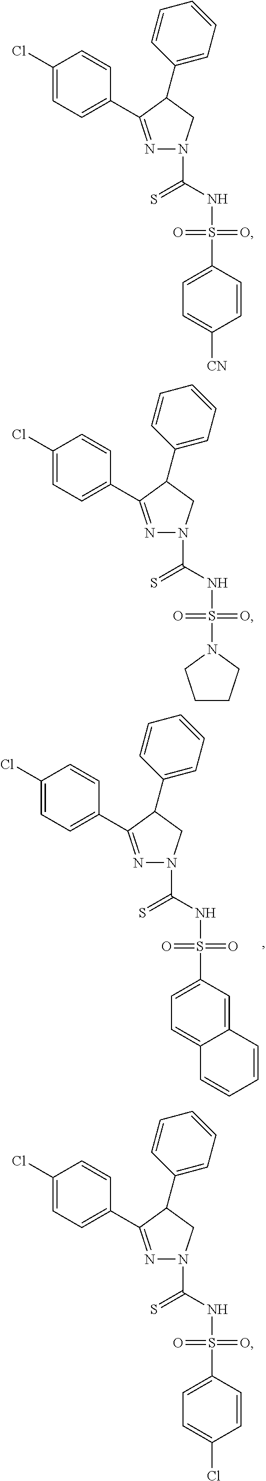

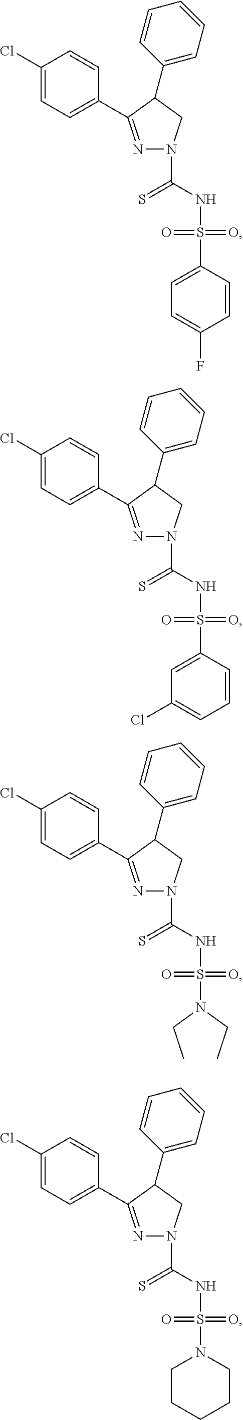

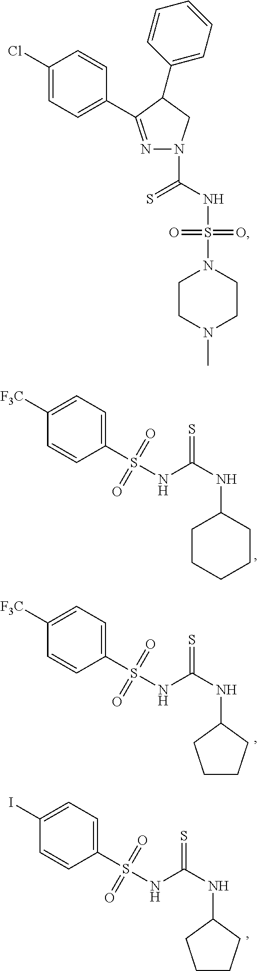

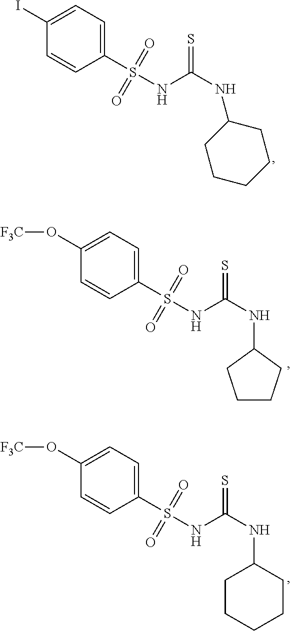

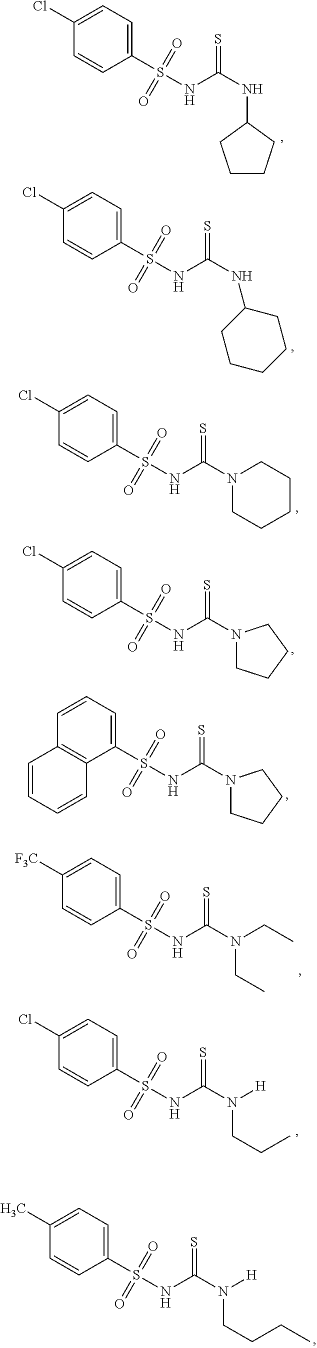

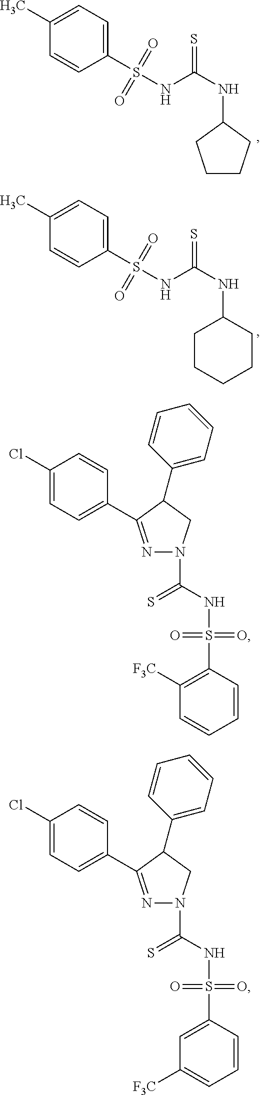

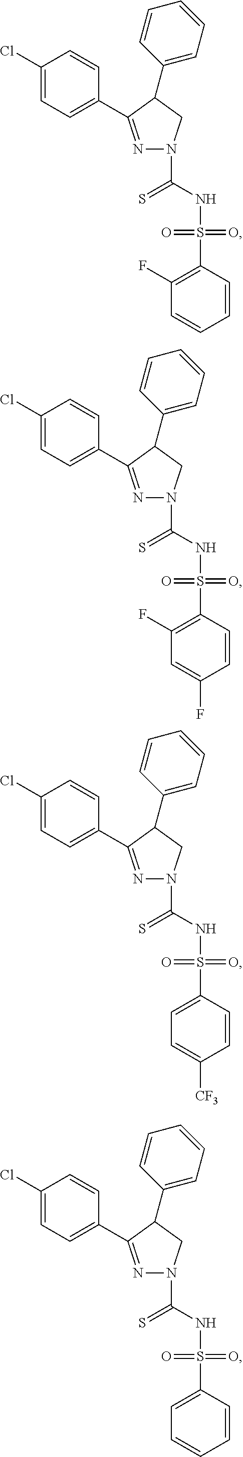

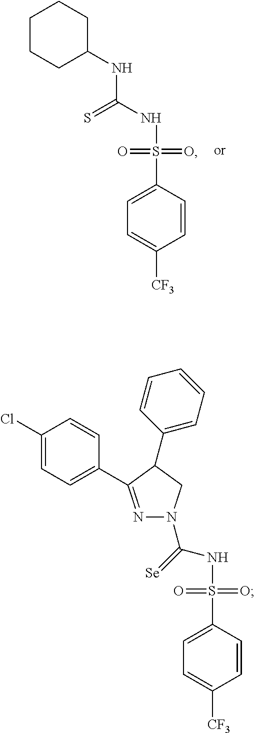

wherein, when the compound has one or more stereocenters, the compound of Formula II can be present as an (R) enantiomer at the one or more stereocenters, an (S) enantiomer at the one or more stereocenters, a mixture of (R) and (S) enantiomers at the one or more stereocenters, or any combination thereof.

### Method for Making an Alkylated Sulfonyl Chalcogenide Urea

Also disclosed herein is a method for making an alkylated sulfonyl chalcogenide urea, the method including at least the steps of:
(a) contacting a sulfonyl chalcogenide urea according to any one of aspects 25-29 with an alkylating agent having Formula IV;

R₃—Z Formula IV;
wherein Z is selected from OTf, Oms, OTs, Br, Cl, and I; and
wherein R₃ is a substituted or unsubstituted linear or branched C₁-C₁₀ alkyl or cycloalkyl, C₃-C₁₀ substituted or unsubstituted aryl or heteroaryl, or a modified or unmodified amino acid; and
(b) refluxing the sulfonyl chalcogenide urea with the alkylating agent.

In one aspect, the alkylating agent can be 1-butynyl bromide, methyl(S)-3-bromo-2-methylpropanoate, (S)-1-bromo-2-methylbutane, bromoacetamide, bromoacetic acid, methyl iodide, (2-bromoacetamido)methyl acetate,

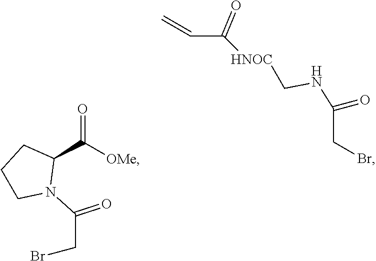

or any combination thereof. Also disclosed herein are alkylated sulfonyl chalcogenide ureas produced by the disclosed methods.

### Alkylated Sulfonyl Chalcogenide Ureas

In one aspect, disclosed herein is an alkylated sulfonyl chalcogenide urea having Formula V:

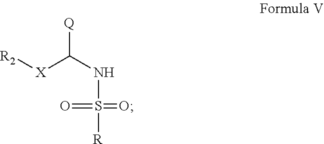

wherein R₂ is a substituted or unsubstituted linear or branched C₁-C₁₀ alkyl or cycloalkyl, C₃-C₁₀ substituted or unsubstituted aryl or heteroaryl, or a modified or unmodified amino acid;
wherein X is O, sulfur, SO, SO₂, selenium, SeO, SeO₂, or tellurium;
wherein Q is selected from a substituted or unsubstituted amine, substituted or unsubstituted heterocycloalkyl group, or a substituted or unsubstituted heteroaryl group,
or any combination thereof, provided Q is not

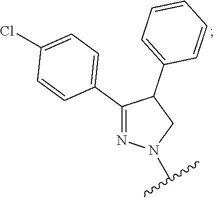

wherein R is selected from a substituted or unsubstituted amine, a substituted or unsubstituted alkyl group, cycloalkyl group, heterocycloalkyl group, or a substituted or unsubstituted aromatic group, or any combination thereof.

In another aspect, Q, R, and X are as defined above, and R₂ can be selected from:

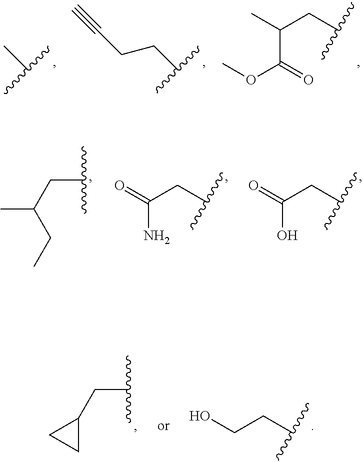

In still another aspect, the alkylated sulfonyl chalcogenide urea of Formula V can be selected from

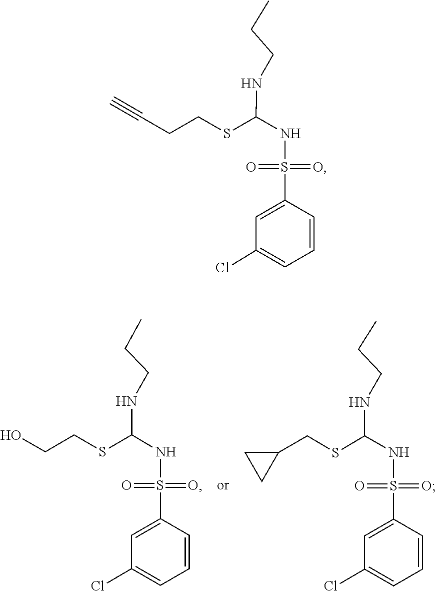 [SMILES: C#CCCSC(NCCC)NS(=O)(=O)c1cccc(Cl)c1 ; CCCNC(NS(=O)(=O)c1cccc(Cl)c1)SCCO ; "or" ; CCCNC(NS(=O)(=O)c1cccc(Cl)c1)SCC1CC1]

and
wherein, when the compound has one or more stereocenters, the compound of Formula II can be present as an (R) enantiomer at the one or more stereocenters, an (S) enantiomer at the one or more stereocenters, a mixture of (R) and (S) enantiomers at the one or more stereocenters, or any combination thereof.

In one aspect, disclosed herein is a method for synthesizing a substituted guanidine from a sulfonyl chalcogenide urea, the method including at least the step of contacting a sulfonyl chalcogenide urea as described herein with a compound having Formula VI

R₄—NH₂ Formula VI;
wherein R₄ is a substituted or unsubstituted linear or branched C1-C10 alkyl or cycloalkyl, C3-C10 substituted or unsubstituted aryl or heteroaryl, or a modified or unmodified amino acid.

Also disclosed herein are substituted guanidines prepared according to the disclosed methods.

### Exemplary Syntheses and Compounds

Scheme 1 shows a generic scheme for producing, in one exemplary aspect, 3,4-diarylpyrazoline sulfonyl thioureas according to the present disclosure.

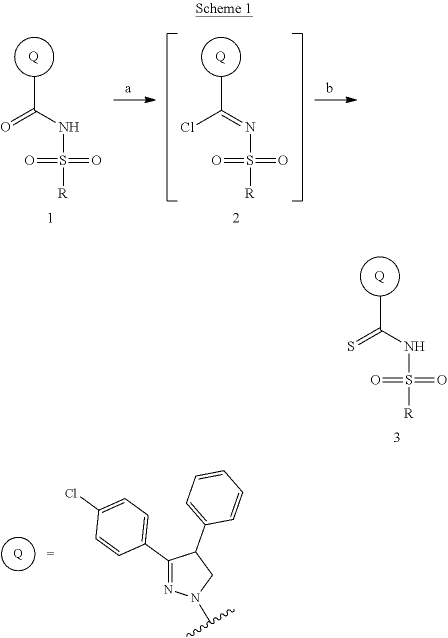

In some aspects, Q can have a chiral center. In a further aspect, the chiral center retains its arrangement of substituents during the reactions disclosed herein. In another aspect, reagents useful in performing step (a) can include POCl₃, N,N-diisopropylethylamine (DIPEA), and toluene under reflux conditions.

In another aspect, reagents useful in pursuing step (b) can include a solvent, a “Bunte” agent, and, in some aspects, an additive. In one aspect, the solvent can be methanol, water, ethanol, dioxane, toluene, dimethylformamide, and combinations thereof. In another aspect, the “Bunte” agent can be Na₂S₂O₃ or K₂S₂O₃. In still another aspect, the additive can be triethylamine. In any of these aspects, the reaction is carried out for about 3 hours.

Scheme 2 shows a specific example of a synthesis according to the present disclosure, where reagents for steps (a) and (b) are as described above.

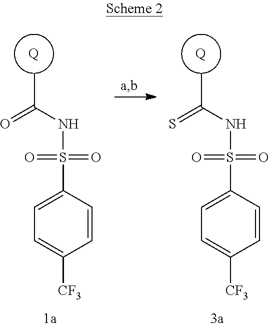

Scheme 3 shows a generic scheme for producing, in one exemplary aspect, general sulfonyl thioureas according to the present disclosure.

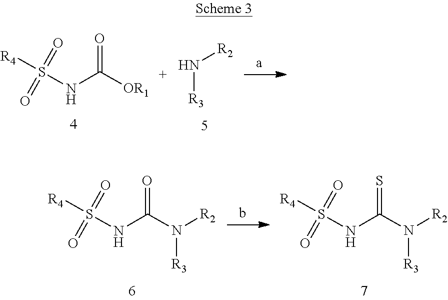

In one aspect, reagents useful in performing step (a) can include POCl₃, N,N-diisopropylethylamine (DIPEA), and toluene under reflux conditions.

In another aspect, reagents useful in pursuing step (b) can include a solvent, a “Bunte” agent, and, in some aspects, an additive. In one aspect, the solvent can be methanol, water, ethanol, dioxane, toluene, dimethylformamide, and combinations thereof. In another aspect, the “Bunte” agent can be Na₂S₂O₃ or K₂S₂O₃. In still another aspect, the additive can be triethylamine. In any of these aspects, the reaction is carried out for about 3 hours.

In any of these aspects, R₁ can be an alkyl group such as, for example, methyl or t-butyl. Additional exemplary compounds and non-limiting examples of R₂, R₃, and R₄ substituents are provided in Table 1 below:

TABLE 1
Exemplary Sulfonyl Ureas

| | |
|---|---|
| 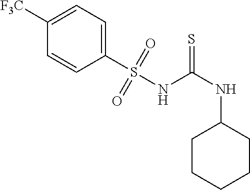 [SMILES: O=S(=O)(NC(=S)NC1CCCCC1)c1ccc(C(F)(F)F)cc1] | 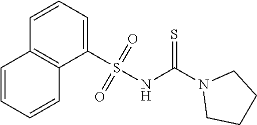 [SMILES: O=S(=O)(NC(=S)N1CCCC1)c1cccc2ccccc12] |
| 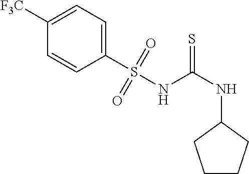 [SMILES: O=S(=O)(NC(=S)NC1CCCC1)c1ccc(C(F)(F)F)cc1] | 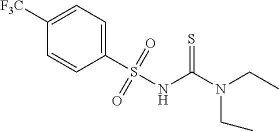 [SMILES: CCN(CC)C(=S)NS(=O)(=O)c1ccc(C(F)(F)F)cc1] |
| 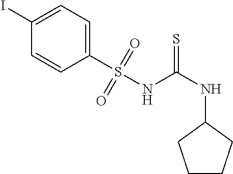 [SMILES: O=S(=O)(NC(=S)NC1CCCC1)c1ccc(I)cc1] | 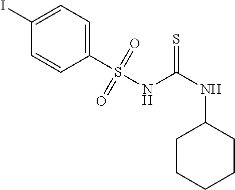 [SMILES: O=S(=O)(NC(=S)NC1CCCCC1)c1ccc(I)cc1] |
| 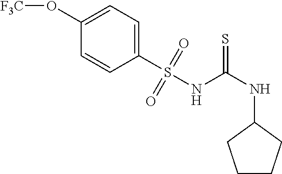 [SMILES: O=S(=O)(NC(=S)NC1CCCC1)c1ccc(OC(F)(F)F)cc1] | 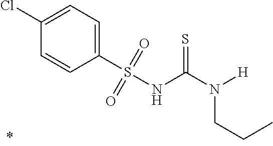 [SMILES: CCCNC(=S)NS(=O)(=O)c1ccc(Cl)cc1] |
| 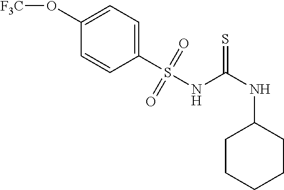 [SMILES: O=S(=O)(NC(=S)NC1CCCCC1)c1ccc(OC(F)(F)F)cc1] | 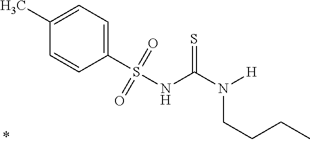 [SMILES: CCCCNC(=S)NS(=O)(=O)c1ccc(C)cc1] |
| 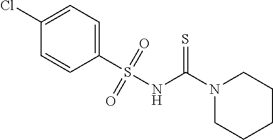 [SMILES: O=S(=O)(NC(=S)N1CCCCC1)c1ccc(Cl)cc1] | 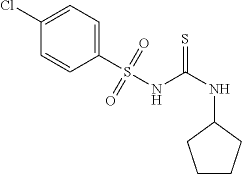 [SMILES: O=S(=O)(NC(=S)NC1CCCC1)c1ccc(Cl)cc1] |
| 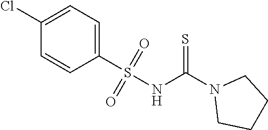 [SMILES: O=S(=O)(NC(=S)N1CCCC1)c1ccc(Cl)cc1] | 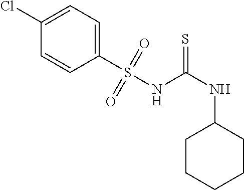 [SMILES: O=S(=O)(NC(=S)NC1CCCCC1)c1ccc(Cl)cc1] |
| 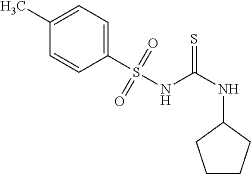 [SMILES: Cc1ccc(S(=O)(=O)NC(=S)NC2CCCC2)cc1] | 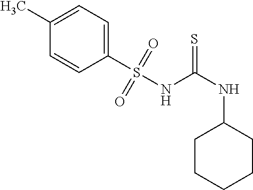 [SMILES: Cc1ccc(S(=O)(=O)NC(=S)NC2CCCCC2)cc1] |

Compounds denoted by an asterisk in Table 1 represent commercially available sulfonylurea drugs chlorpropamide and tolbutamide, respectively. Thus, in one aspect, disclosed herein is a new method for synthesizing these and related compounds, wherein the new method proceeds under mild conditions and using reagents that are not malodorous.

In another aspect, disclosed herein is a method for synthesizing sulfonyl selenourea compounds. Scheme 4 shows a generic scheme for producing, in one exemplary aspect, general sulfonyl thioureas according to the present disclosure.

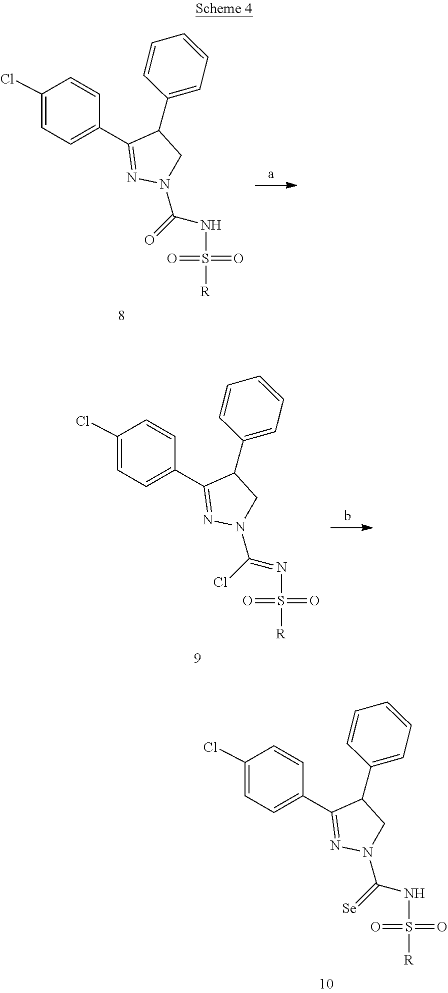

In one aspect, reagents useful in performing step (a) can include POCl₃, N,N-diisopropylethylamine (DIPEA), (POCl₃/DMAP in CH₂Cl₂ reflux) and, optionally, toluene or another solvent under reflux conditions.

In another aspect, reagents useful in pursuing step (b) can include a solvent, a “seleno Bunte” agent, and, in some aspects, an additive. In one aspect, the solvent can be methanol, water, ethanol, dioxane, toluene, dimethylformamide, and combinations thereof. In another aspect, the “seleno Bunte” agent can be K₂Se₂O₃ or Na₂SeSO₃. In still another aspect, the additive can be triethylamine. In any of these aspects, the reaction is carried out for about 3 hours.

In some aspects, R can be 4-trifluoromethylphenyl or another R group as disclosed herein.

In one aspect, provided herein is a reaction of the sulfonyl ureas (1) with PCl₅ or POCl₃/DIPEA giving intermediate imidoyl chlorides which, upon treatment with Na₂S₂O₃, gave the putative Bunte intermediates that promptly decomposed to the sulfonylthioureas in excellent yields (Scheme 1). In another aspect, the disclosed one-pot, two-step protocol represents an improvement over previous attempts to generate sulfonyl thioureas from refluxing xylenes in presence of P₂S₅ and under microwave conditions. In still another aspect, this methodology has wide applicability in generation of thiourea and isothiourea analogs.

In some aspects, the chalcogenide sulfonylureas disclosed herein can have one or more stereocenters. In one aspect, when one or more stereocenters are present, the one or more stereocenters can have an (R) configuration, an (S) configuration, or can be a mixture of (R) and (S) enantiomers at the one or more stereocenters, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.
Aspect 1. A method for synthesizing a sulfonyl chalcogenide urea, the method comprising
(a) admixing a sulfonyl urea with a source of a leaving group to produce a first composition; and
(b) admixing the first composition with a compound having the formula M₂X₂O₃·mH₂O or M₂XJO₃·mH₂O to produce the sulfonyl chalcogenide urea, wherein M is selected from K, Na, or Li;
wherein X and J are independently selected from S, Se, or Te; and wherein
m is from 0 to 6.
Aspect 2. The method of aspect 1, wherein the leaving group comprises OTs, OTf, OMs, or a halide.
Aspect 3. The method of aspect 2, wherein the halide is chloride, bromide, or iodide.
Aspect 4. The method of any of aspects 1-3 comprising:
admixing a sulfonyl urea of Formula I with a source of a leaving group to form the first composition:

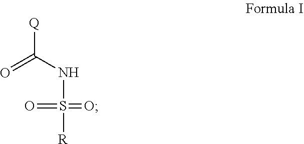

and
admixing the first composition with the compound having the formula M₂X₂O₃·mH₂O or M₂XJO₃·mH₂O to form the sulfonyl chalcogenide urea having Formula II:

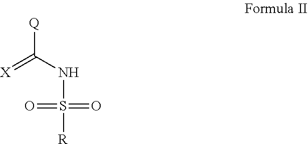

wherein X and J are independently sulfur, selenium, or tellurium;
wherein Q is selected from a substituted or unsubstituted amine, substituted or unsubstituted heterocycloalkyl group, or a substituted or unsubstituted heteroaryl group, or any combination thereof; and
wherein R is selected from a substituted or unsubstituted amine, a substituted or unsubstituted alkyl group, cycloalkyl group, heterocycloalkyl group, or a substituted or unsubstituted aromatic group, or any combination thereof.
Aspect 5. The method of any one of the preceding aspects, wherein the source of leaving group comprises a halogenating agent.
Aspect 6. The method of aspect 5, wherein the halogenating agent comprises a phosphorus halide, a dialkyl chloroiminium salt, a diaryl chloroiminium salt, an oxalyl halide, a carbonyl dihalide, or any combination thereof.
Aspect 7. The method of aspect 6, wherein the phosphorus halide has the formula PW₃, POW₃, or PW₅, wherein W is selected from Cl, Br, or I.
Aspect 8. The method of any one of the preceding aspects, wherein step (a) further comprises admixing a base with the sulfonyl urea and the source of the leaving group.
Aspect 9. The method of aspect 8, wherein the base comprises N,N-diisopropylethylamine (DIPEA), triethylamine, 4-dimethylaminopyridine (DMAP), imidazole, benzimidazole, guanidine, ammonium hydroxide, pyridine, lutidine, sodium hydroxide, potassium hydroxide, 1,8-diazabicyclo[5.4.0]undec-7-ene (DBU), or any combination thereof.
Aspect 10. The method of any one of the preceding aspects, wherein step (a) is carried out in a solvent.
Aspect 11. The method of aspect 10, wherein the solvent comprises toluene, chlorobenzene, dimethylformamide, dichloromethane, dichloroethane, xylenes, or any combination thereof.
Aspect 12. The method of any one of the preceding aspects, wherein the compound having the formula M₂X₂O₃·mH₂O or M₂XJO₃·mH₂O comprises Na₂S₂O₃19 5H₂O, Na₂S₂O₃, K₂S₂O₃, K₂Se₂O₃, or Na₂SeSO₃.
Aspect 13. The method of aspect 12, wherein X is sulfur and the compound having the formula M₂X₂O₃·mH₂O comprises Na₂S₂O₃ or K₂S₂O₃.
Aspect 14. The method of aspect 12, wherein X is selenium and the compound having the formula M₂X₂O₃·mH₂O or M₂XJO₃·mH₂O comprises K₂Se₂O₃ or Na₂SeSO₃.
Aspect 15. The method of any one of the preceding aspects, wherein step (b) further comprises admixing an additive with the first composition and the compound having the formula M₂X₂O₃·mH₂O or M₂XJO₃·mH₂O.
Aspect 16. The method of aspect 15, wherein the additive comprises triethylamine, Li₂CO₃, Na₂CO₃, K₂CO₃, MgCO₃, CaCO₃, BaCO₃, LiHCO₃, NaHCO₃, KHCO₃, Mg(HCO₃)₂, Ca(HCO₃)₂, tetrabutylammonium bromide, tetramethylammonium chloride, or any combination thereof.
Aspect 17. The method of any one of the preceding aspects, wherein step (b) is carried out in a solvent.
Aspect 18. The method of aspect 17, wherein the solvent comprises methanol, water, ethanol, dioxane, tetrahydrofuran, dimethylformamide, acetone, acetonitrile, DMSO, toluene, isopropyl alcohol, xylenes, ethylene glycol, chlorobenzene, or any combination thereof.
Aspect 19. The method of any one of the preceding aspects, wherein the method is carried out at a temperature of from room temperature to 140° C.
Aspect 20. The method of any one of the preceding aspects, wherein Q is selected from:

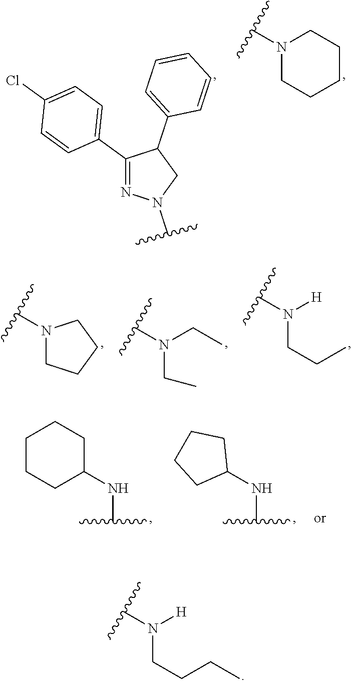

Aspect 21. The method of any one of the preceding aspects, wherein R is selected from:

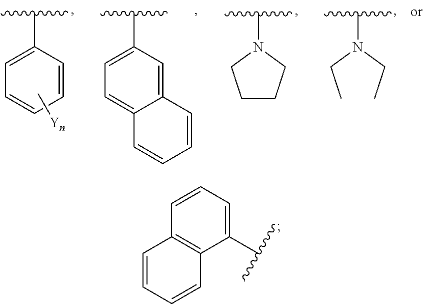

wherein each Y is independently selected from hydrogen, substituted or unsubstituted linear or branched C1-C10 alkyl, nitro, substituted or unsubstituted amino, halogen, cyano, alkoxy, thiol, phosphonate, haloalkyl, or haloalkoxy;
and wherein n is an integer from 1 to 5.
Aspect 22. The method of any one of the preceding aspects, wherein prior to step (a), the sulfonyl urea is produced in situ by reacting a sulfonyl carbamate or an isocyanate with a substituted or unsubstituted amine.
Aspect 23. The method of aspect 22, wherein the sulfonyl carbamate is a compound of Formula III or Formula IIIa and the substituted or unsubstituted amine is a compound having formula Q-H:

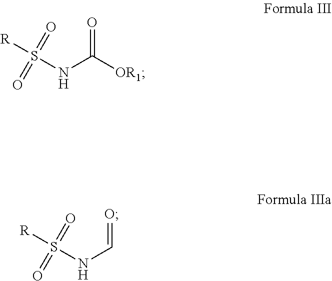

wherein R₁, when present, comprises a substituted or unsubstituted linear or branched alkyl, aryl, or aralkyl group; and
wherein R is selected from a substituted or unsubstituted amine, a substituted or unsubstituted alkyl group, cycloalkyl group, heterocycloalkyl group, or a substituted or unsubstituted aromatic group, or any combination thereof.
Aspect 24. The method of aspect 23, wherein R₁ is methyl, tert-butyl, or phenyl.
Aspect 25. A sulfonyl chalcogenide urea produced by the method of any one of aspects 1-24.
Aspect 26. A sulfonyl chalcogenide urea having Formula II:

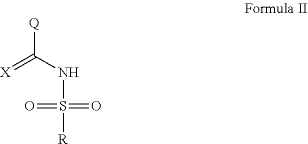

wherein X is sulfur, selenium, or tellurium;
wherein Q is selected from a substituted or unsubstituted amine, substituted or unsubstituted heterocycloalkyl group, or a substituted or unsubstituted heteroaryl group, or any combination thereof;
wherein R is selected from a substituted or unsubstituted amine, a substituted or unsubstituted alkyl group, cycloalkyl group, heterocycloalkyl group, or a substituted or unsubstituted aromatic group, or any combination thereof; and
wherein, when the compound has one or more stereocenters, the compound of Formula II can be present as an (R) enantiomer at the one or more stereocenters, an (S) enantiomer at the one or more stereocenters, a mixture of (R) and (S) enantiomers at the one or more stereocenters, or any combination thereof.
Aspect 27. The sulfonyl chalcogenide urea of aspect 26, wherein Q is selected from:

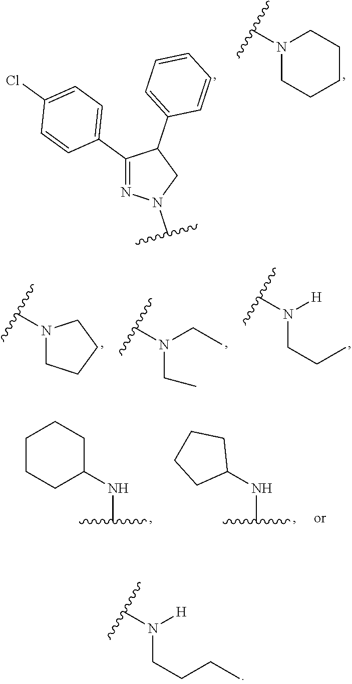

Aspect 28. The sulfonyl chalcogenide urea of aspect 26 or 27, wherein R is selected from:

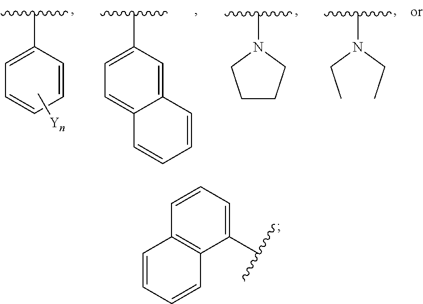

wherein each Y is independently selected from hydrogen, substituted or unsubstituted linear or branched C1-C10 alkyl, nitro, substituted or unsubstituted amino, halogen, cyano, alkoxy, thiol, phosphonate, haloalkyl, or haloalkoxy; and
wherein n is an integer from 1 to 5.
Aspect 29. The sulfonyl chalcogenide urea of any one of aspects 26-28, wherein the compound of Formula II is selected from:

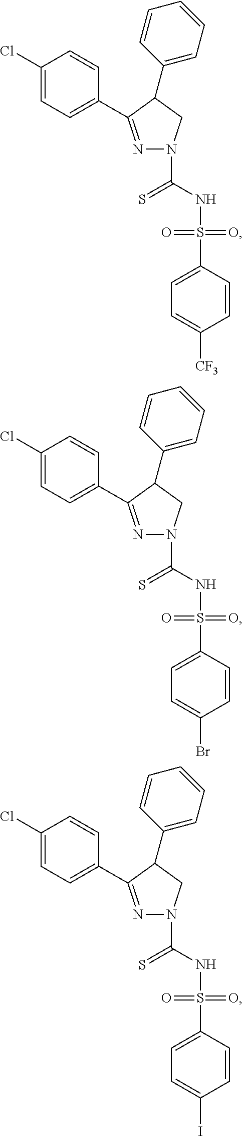

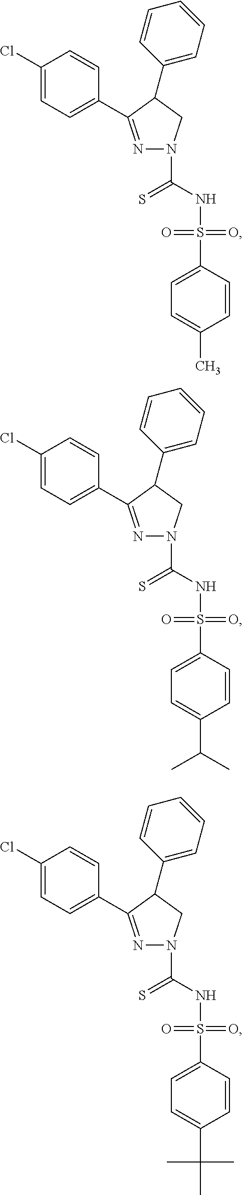

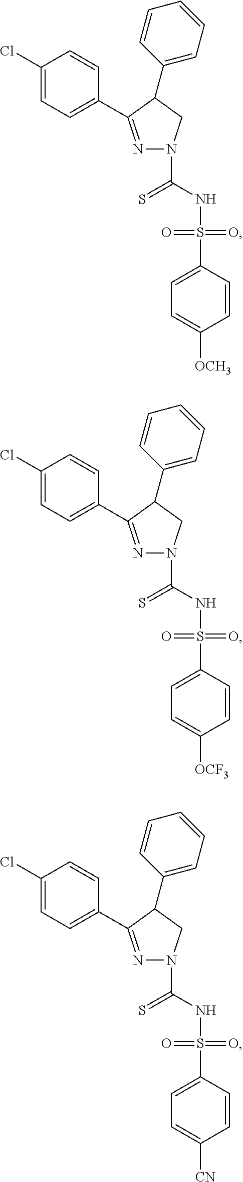

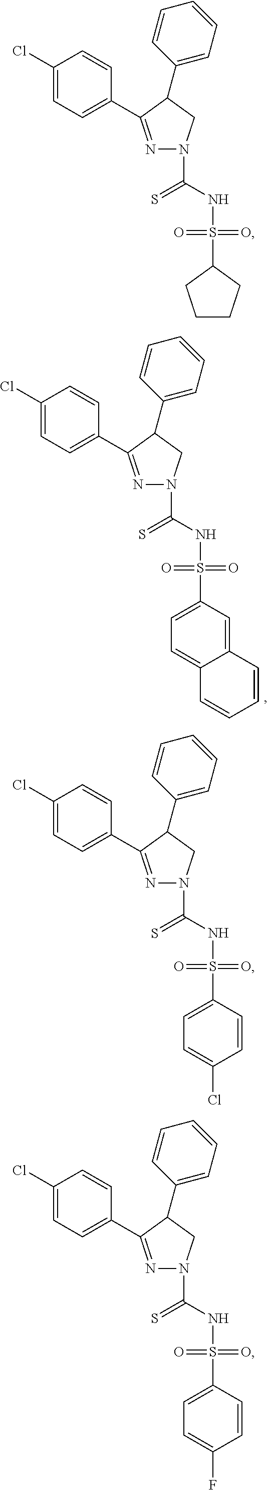

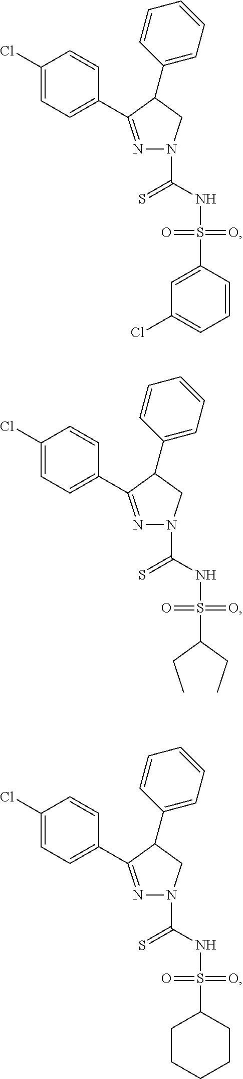

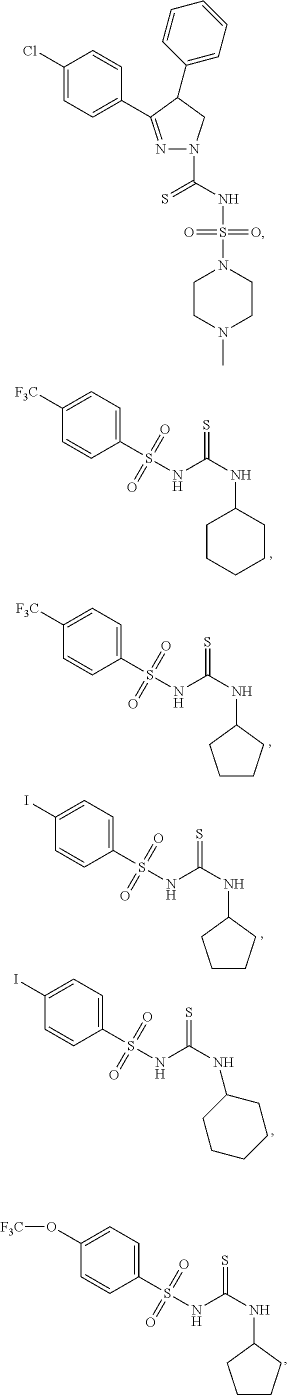

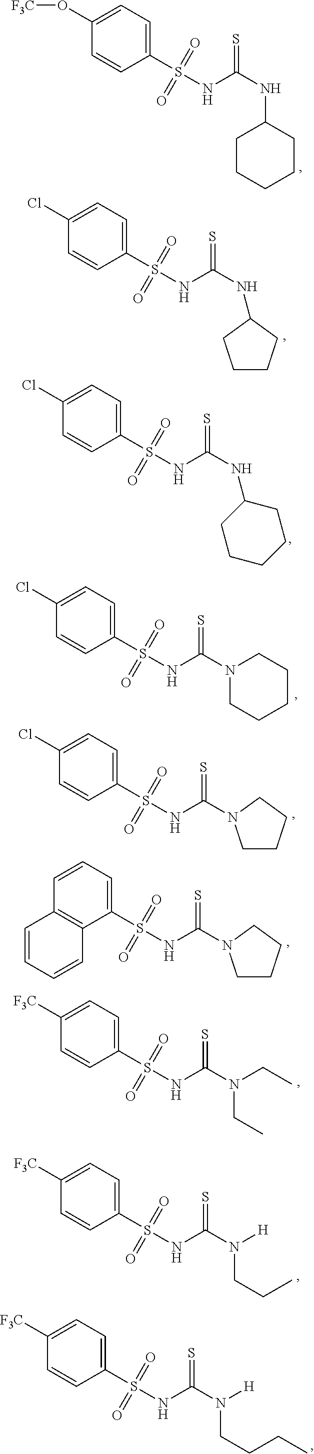

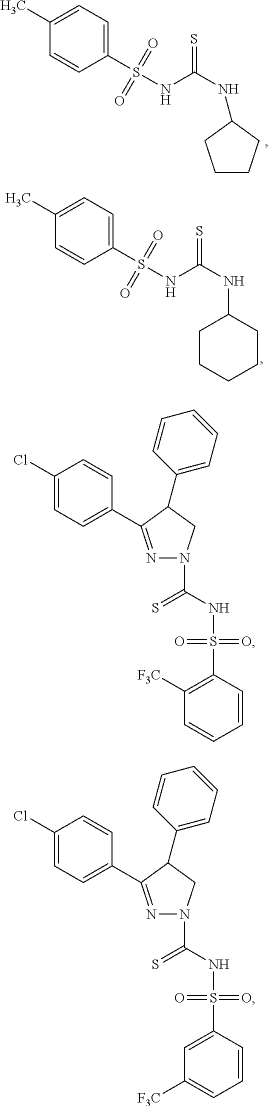

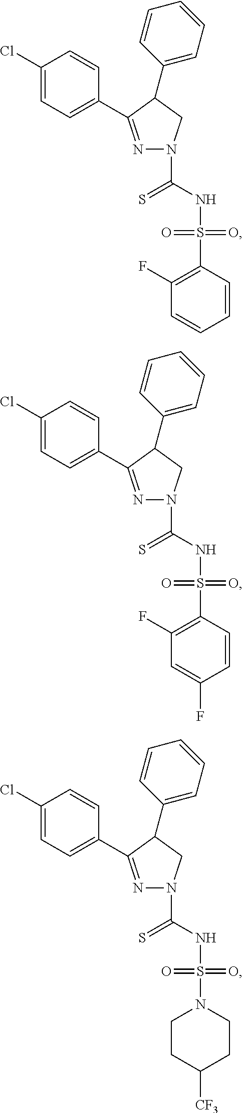

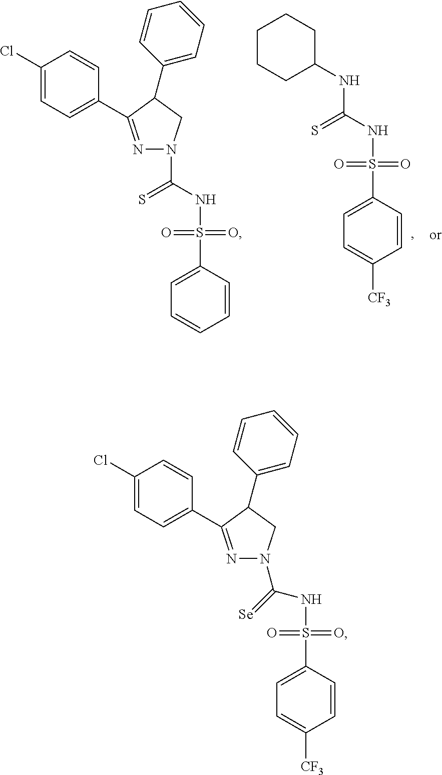

wherein, when the compound has one or more stereocenters, the compound of Formula II can be present as an (R) enantiomer at the one or more stereocenters, an (S) enantiomer at the one or more stereocenters, a mixture of (R) and (S) enantiomers at the one or more stereocenters, or any combination thereof.
Aspect 30. A method for making an alkylated sulfonyl chalcogenide urea, the method comprising:
(a) contacting a sulfonyl chalcogenide urea according to any one of aspects 25-29 with an alkylating agent having Formula IV;

R₃—Z Formula IV;
wherein Z is selected from OTf, Oms, OTs, Br, Cl, and I; and
wherein R₃ is a substituted or unsubstituted linear or branched C₁-C₁₀ alkyl or cycloalkyl, C₃-C₁₀ substituted or unsubstituted aryl or heteroaryl, or a modified or unmodified amino acid; and
(b) refluxing the sulfonyl chalcogenide urea with the alkylating agent.
Aspect 31. The method of aspect 30, wherein the alkylating agent comprises 1-butynyl bromide, methyl(S)-3-bromo-2-methylpropanoate, (S)-1-bromo-2-methylbutane, bromoacetamide, bromoacetic acid, methyl iodide, (2-bromoacetamido)methyl acetate,

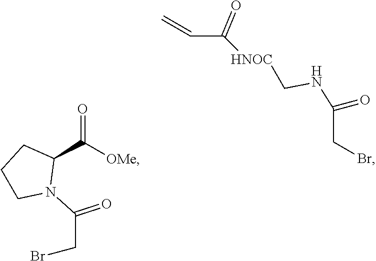

or any combination thereof.
Aspect 32. An alkylated sulfonyl chalcogenide urea produced by the method of aspect 30 or 31.
Aspect 33. An alkylated sulfonyl chalcogenide urea having Formula V:

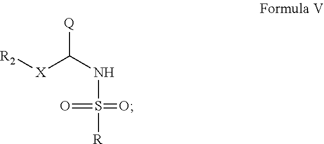

wherein R₂ is a substituted or unsubstituted linear or branched C1-C10 alkyl or cycloalkyl, C3-C10 substituted or unsubstituted aryl or heteroaryl, or a modified or unmodified amino acid;
wherein X is O, sulfur, SO, SO₂, selenium, SeO, SeO₂, or tellurium;
wherein Q is selected from a substituted or unsubstituted amine, substituted or unsubstituted heterocycloalkyl group, or a substituted or unsubstituted heteroaryl group,
or any combination thereof, provided Q is not

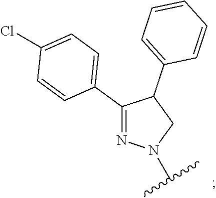

wherein R is selected from a substituted or unsubstituted amine, a substituted or unsubstituted alkyl group, cycloalkyl group, heterocycloalkyl group, or a substituted or unsubstituted aromatic group, or any combination thereof; and
wherein, when the compound has one or more stereocenters, the compound of Formula V can be present as an (R) enantiomer at the one or more stereocenters, an (S) enantiomer at the one or more stereocenters, a mixture of (R) and (S) enantiomers at the one or more stereocenters, or any combination thereof.
Aspect 34. The alkylated sulfonyl chalcogenide urea of aspect 33, wherein Q is selected from:

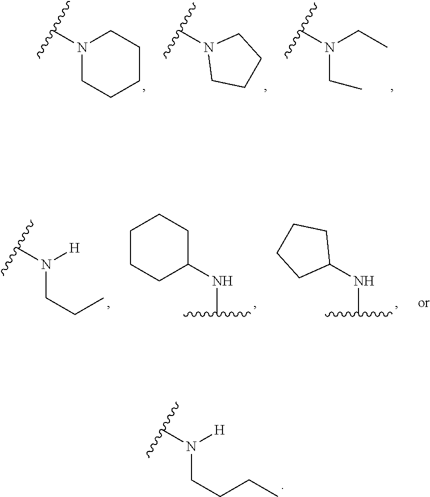

Aspect 35. The alkylated sulfonyl chalcogenide urea of aspect 33 or 34, wherein R is selected from:

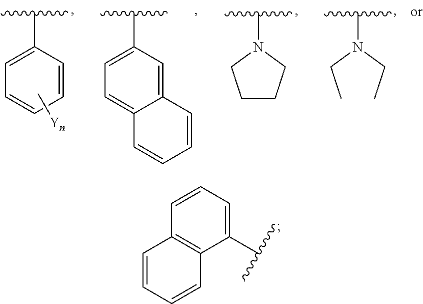

wherein each Y is independently selected from hydrogen, substituted or unsubstituted linear or branched C1-C10 alkyl, nitro, substituted or unsubstituted amino, halogen, cyano, alkoxy, thiol, phosphonate, haloalkyl, or haloalkoxy; and
wherein n is an integer from 1 to 5.
Aspect 36. The alkylated sulfonyl chalcogenide urea of any one of aspects 33-35, wherein R₂ is selected from:

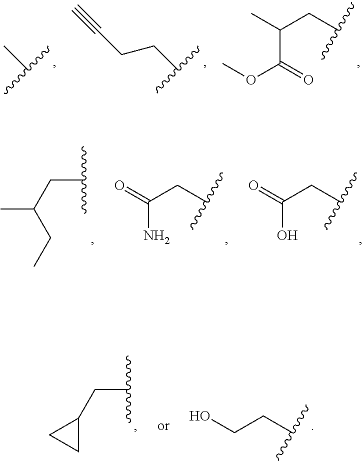

Aspect 37. The alkylated sulfonyl chalcogenide urea of any one of aspects 33-36, wherein the compound of Formula V is selected from:

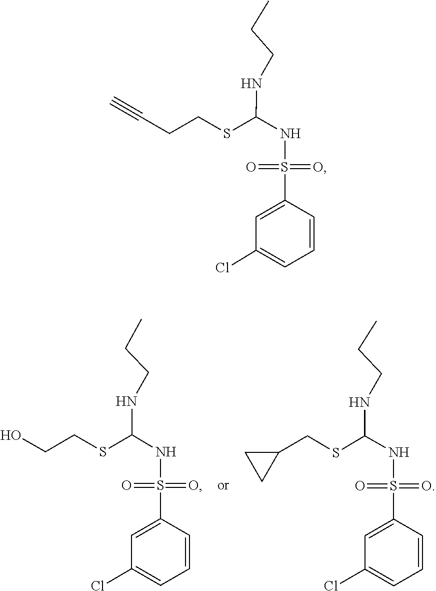 [SMILES: C#CCCSC(NCCC)NS(=O)(=O)c1cccc(Cl)c1 ; CCCNC(NS(=O)(=O)c1cccc(Cl)c1)SCCO ; "or" ; CCCNC(NS(=O)(=O)c1cccc(Cl)c1)SCC1CC1]

Aspect 38. A method for synthesizing a substituted guanidine from a sulfonyl chalcogenide urea, the method comprising contacting a sulfonyl chalcogenide urea according to any one of aspects 25-29 with a compound having Formula VI

R₄—NH₂ Formula VI;

wherein R₄ is a substituted or unsubstituted linear or branched C1-C10 alkyl or cycloalkyl, C3-C10 substituted or unsubstituted aryl or heteroaryl, or a modified or unmodified amino acid.
Aspect 39. A substituted guanidine prepared according to the method of aspect 38.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

Reagents available commercially were purchased and used as is. Starting materials not available commercially were synthesized based on published procedures. Melting points were determined on an Optimelt (SRS) instrument and are uncorrected. Proton (1H NMR) spectra were recorded on a Bruker 500 or 800 MHZ spectrometer in CDCl₃ or DMSO-d₆ (unless otherwise noted) with the values given in ppm (TMS as internal standard) and J (Hz) assignments of 1H resonance coupling. Mass spectra (HRMS) were recorded on a VG 7070E spectrometer or a JEOL SX102a mass spectrometer. Thin layer chromatography (TLC) analyses were carried out on silica gel (Miles Scientific or Analtech) GHLF 0.25 mm plates using various gradients of CHCl₃/MeOH containing 1% NH₄OH or gradients of EtOAc:n-hexane (30-50%). Visualization was accomplished under UV light or by staining in an iodine chamber. Flash column chromatography was performed on a Teledyne ISCO Combiflash system with and without Purlon mass detector. Product yields are unoptimized unless indicated.

All compounds isolated after purification had ≥95% purity unless indicated. Purity and structural confirmation were analyzed by a combination of TLC, 1H-NMR, 13C-NMR (where available), LC/MS, high-resolution mass spectrometry, and X-ray crystallography (where indicated). LC-MS detection was carried out on Agilent 1200 using Luna C18 3 μm (3×75 mm) or Poroshell 120 EC-C18 (3×50 mm/2.7 μm particle size). The mobile phase was 4% to 100% acetonitrile (0.05% TFA) standard gradient or, in some experiments, 50% to 98% acetonitrile (1% formic acid) over 8 minutes with a standard gradient. The LC-MS chromatogram showed the correct molecular (MH⁺) ion as well as a single UV peak (254 nm). Chiral HPLC was carried out using a Whelko (R, R) chiral column using the method as indicated for specific compounds. X-ray results were obtained using the X-ray facility at Georgetown University.

### Example 2: Reaction Conditions

The synthesis of 3,4-diarylpyrazolines carbothioamide analog 3a was first explored using starting material 1a for optimizing the reaction conditions. In a step wise protocol, treatment of carboxamide compound (1a, 1 eq)) POCl₃/DIPEA (1.3 eq/1.4 eq) (or PCIs) led to formation of the intermediate imidoyl chloride. Upon subsequent treatment with Na₂S₂O₃ (2 eq) provided the carbothioamide analogs in excellent yields. (Scheme 1). Methanol/H₂O at 90° C. worked best and the thiourea analog could be generated with 3 h at 1 mmol scale. DMF/H₂O and ethanol/H₂O were also acceptable solvents for the reaction. The reaction proceeded slowly in aqueous toluene and dioxane. Water was deemed essential for solubilization of the inorganic reactant, although the reaction did proceed in wet methanol likely due to adventitious water.

The in situ Bunte salts underwent immediate decomposition to the thiol/thiourea compounds and the 3,4-dihydropyrazoline Bunte salt was not isolated. Evaporation of methanol followed by extraction of the organic compound into dichloromethane and subsequent trituration in isopropyl alcohol gave the pure thiourea in excellent yield.

For initial syntheses of 3,4-diarylpyrazoline sulfonyl thioureas, certain reaction conditions (e.g., solvent, Bunte agent, additive, and reaction time) were optimized; these are presented in Table 2 below:

TABLE 2
Reaction Optimization

| Solvent | Bunte Agent | Additive | Reaction Time (h) |
|---|---|---|---|
| Methanol | Na₂S₂O₃ | — | 3 |
| Methanol:H₂O | Na₂S₂O₃ | — | 3 |
| Ethanol:H₂O | Na₂S₂O₃ | — | 3 |
| Dioxane:H₂O | Na₂S₂O₃ | — | 3 |
| Toluene:H₂O | Na₂S₂O₃ | — | 3 |
| DMF:H₂O | Na₂S₂O₃ | — | 3 |
| Methanol:H₂O | K₂S₂O₃ | — | 3 |
| Methanol:H₂O | Na₂S₂O₃ | Triethylamine | 3 |

Additional reaction conditions were screened using compound 1a on a 1 mmol scale; results are provided in Table 3 below:

TABLE 3
Reaction Optimization

| | | | Temperature | |
|---|---|---|---|---|
| Entry | Reagent | Solvent^{a} | (° C.) | Time/conversion^{b} |
| 1 | POCl₃/DIPEA | Toluene | 110 | 1.5 h/complete (, 5% |
| | | | | decomposition) |
| 2 | POCl₃/DIPEA | Toluene | 95 | 2 h/complete, clean (>95%) |
| 3 | POBr₃/DIPEA | Toluene | 110 | 1.5 h/complete with ~50% |
| | | | | decomposition |
| 4 | POBr₃/DIPEA | Toluene | 85 | 2 h/complete with ~30% |
| | | | | decomposition |
| 5 | PCl₅ | Chlorobenzene | 140 | 1.5 h/up to 15% decomposition |

^{a}Water was used at maximum 10% solvent combination.
^{b}Conversion checked by LCMS.

As described in Table 3, the conversion of 1a to 3a proceeds under flexible temperature conditions and decreasing equivalents of Na₂S₂O₃ at higher temperatures without a significant loss of yield. The alkylation conditions are optimum at 90-95° C. with thiosulfate displacement carried out under 90-95° C. 2 eq of Na₂S₂O₃.

In general, for substrates 1a-1w, chlorpropamide, tolbutamide and 15, aq. Methanol is the solvent of choice. For SUs, chlorpropamide, tolbutamide and 15<10% of the products arising from MeOH displacement is seen (LCMS) at 90° C. with 2 eq of Na₂S₂O₃. For the alkylation procedure, in situ trapping of Bunte salt without thiourea isolation is the method of choice. The alkylation of thiourea of the type 3a proceeds to give products in good yields even under aq. methanol or dioxane. When the thiourea was isolated and alkylation attempted in methanol/95° C. the reaction proceeded sluggishly (Mel) or barely traces of product are seen in case of alkylating agents like above.

Further reaction conditions were screened using compound 1a on a 100 mg/l mmol scale; results are provided in Table 4 below:

TABLE 4
Reaction Optimization

| | Reagent/ | | Temperature | Time to complete |
|---|---|---|---|---|
| Entry | Equivalents | Solvent^{a} | (° C.) | conversion^{b}/% Yield from 1a^{c, d} |
| 1 | Na₂S₂O₃ (5 eq) | MeOH-H₂O | RT | 12 h/^{c} 85% |
| 2 | Na₂S₂O₃ (5 eq) | MeOH-H₂O | 55 | 2 h/^{c} 80% |
| 3 | Na₂S₂O₃ (3 eq) | MeOH-H₂O | 65 | 2 h/NA |
| 4 | Na₂S₂O₃ (2 eq) | MeOH-H₂O | 85 | 30 min/^{c} 78% |
| 5 | K₂S₂O₃ (2 eq) | MeOH-H₂O | 85 | 20 min/^{c} NA |
| 6 | Na₂S₂O₃ (2 eq) | MeOH-H₂O | 90 | 20 min/^{d} 91% |
| 7 | Na₂S₂O₃ (5 eq) | MeOH | 55 | 3 h/^{c} 70% |
| 8 | Na₂S₂O₃ (2 eq) | MeOH | 85 | 1 h/^{c} 84% |
| 9 | Na₂S₂O₃ (2 eq) | IPA-H₂O | 90 | 30 min/^{c} NA |
| 10 | Na₂S₂O₃ (2 eq) | EtOH-H₂O | 90 | 20 min/^{c} NA |
| 11 | Na₂S₂O₃ (5 eq) | Dioxane-H₂O | 55 | 3 h/^{c} NA |
| 12 | Na₂S₂O₃ (2 eq) | Dioxane-H₂O | 85 | 30 min/^{d} 72% |
| 13 | Na₂S₂O₃ (2 eq) | Toluene-H₂O | 90 | —/traces |
| 14 | Na₂S₂O₃ (2 eq) | DMF-H₂O | 85 | 30 min/^{d} 68% |
| 15 | Na₂S₂O₃ (2 eq) | Acetonitrile-H₂O | 85 | 1 h/^{c} 68% |
| 16 | Na₂S₂O₃ (2 eq) | H₂O | 95 | Solubility issues/NA |

^{a}Water was used at maximum 10% solvent combination.
^{b}Conversion based on LCMS comparison with intermediate imidylchloride 1b.
^{c}Yield based on work-up/MeOH-IPA (1:1) trituration.
^{d}Yield based on work-up/flash chromatography (40% hexanes/EtOAc).

Conversion of 1a to Se-3a proceeds under conditions of dioxane with aq. Na₂SeSO₃ (pre-formed) temperatures at 90° C. The ‘Seleno-sulfonyl urea’ could not be isolated cleanly but it was observed in LCMS. Prompt trapping by reagents like methyl iodide allowed the clean isolation of seleno-adducts. The alkylation conditions were optimum at 90-95° C. with thiosulfate displacement yielding new seleno-analogs.

### Example 3: Synthesis and Characterization of Sulfonylureas

Sulfonylureas (SU) were synthesized according to General Procedure B and as reported in the literature or procured commercially when available.

General Procedure B: To a mixture of compound 1a (100 mg, 0.20 mmol) in toluene (5 mL), POCl₃ (0.03 mL, 1.5 mmol), was added, followed by the addition of N,N-diisopropylethylamine (DIPEA) (0.03 mL, 1.5 mmol) and the mixture was refluxed for 1.5 h under N₂ atmosphere. The reaction mixture was then cooled, and the excess reagents in toluene were evaporated in vacuo. The imidoyl chloride intermediate was dissolved in methanol (5 mL), to this solution was added dropwise, sodium thiosulfate (5 eq, 158 mg, 1 mmol) dissolved in 0.5 mL water and the reaction was heated to 55° C. for 2 h. Upon completion of reaction as seen by TLC, the reaction mixture was cooled to room temperature and methanol was evaporated. The organic mixture was the extracted into dichloromethane, washed with brine, and dried over Na₂SO₄. The sticky solid was triturated with (50-50/mix of MeOH/IPA) to give compound 3a as a pale white powder (82 mg, 80% yield).

The following sulfonylureas were synthesized according to literature procedures: 3-(4-chlorophenyl)-4-phenyl-N-((4-(trifluoromethyl)phenyl) sulfonyl)-4,5-dihydro-1Hpyrazole-1-carboxamide (1a), 3-(4-chlorophenyl)-4-phenyl-N-((3-(trifluoromethyl)phenyl) sulfonyl)-4,5-dihydro-1Hpyrazole-1-carboxamide (1c), 3-(4-chlorophenyl)-N-((3-chlorophenyl) sulfonyl)-4-phenyl-4,5-dihydro-1H-pyrazole-1-carboxamide (1d), 3-(4-chlorophenyl)-4-phenyl-N-((4-(trifluoromethoxy)phenyl) sulfonyl)-4,5-dihydro-1Hpyrazole-1-carboxamide (1e), N-((4-bromophenyl) sulfonyl)-3-(4-chlorophenyl)-4-phenyl-4,5-dihydro-1H-pyrazole-1-carboxamide (1f), 3-(4-chlorophenyl)-4-phenyl-N-tosyl-4,5-dihydro-1H-pyrazole-1-carboxamide (3g), 3-(4-chlorophenyl)-N-((4-isopropylphenyl) sulfonyl)-4-phenyl-4,5-dihydro-1H-pyrazole-1-carboxamide (1h), N-((4-(tert-butyl)phenyl) sulfonyl)-3-(4-chlorophenyl)-4-phenyl-4,5-dihydro-1H-pyrazole-1-carboxamide (1i), 3-(4-chlorophenyl)-N-(naphthalen-2-ylsulfonyl)-4-phenyl-4,5-dihydro-1H-pyrazole-1-carboxamide (1j), 3-(4-chlorophenyl)-N-((4-fluorophenyl) sulfonyl)-4-phenyl-4,5-dihydro-1H-pyrazole-1-carboxamide (1k), 3-(4-chlorophenyl)-N-((4-iodophenyl) sulfonyl)-4-phenyl-4,5-dihydro-1H-pyrazole-1-carboxamide (11), 3-(4-chlorophenyl)-N-((4-cyanophenyl) sulfonyl)-4-phenyl-4,5-dihydro-1H-pyrazole-1-carboxamide (1m), 3-(4-chlorophenyl)-N-(phenyl) sulfonyl)-4-phenyl-4,5-dihydro-1H-pyrazole-1-carboxamide (1n), 3-(4-chlorophenyl)-N-((2,4-difluorophenyl) sulfonyl)-4-phenyl-4,5-dihydro-1H-pyrazole-1-carboxamide (10), 3-(4-chlorophenyl)-N-((4-chlorophenyl) sulfonyl)-4-phenyl-4,5-dihydro-1Hpyrazole-

1-carboxamide (1q), 3-(4-chlorophenyl)-N-((4-methoxyphenyl) sulfonyl)-4-phenyl-4,5-dihydro-1H-pyrazole-1-carboxamide (1t), 3-(4-chlorophenyl)-N—(N,N-diethylsulfamoyl)-4-phenyl-4,5-dihydro-1H-pyrazole-1-carboxamide (1u), 3-(4-chlorophenyl)-4-phenyl-N-(piperidin-1-ylsulfonyl)-4,5-dihydro-1H-pyrazole-1-carboxamide (1v), 3-(4-chlorophenyl)-4-phenyl-N-((4-(trifluoromethyl) piperidin-1-yl) sulfonyl)-4,5-dihydro-1H-pyrazole-1-carboxamide (1w), 3-(4-chlorophenyl)-N-((4-chlorophenyl) sulfonyl)-4-phenyl-5,6-dihydropyridazine-1 (4H)-carboxamide (11c), N-(Adamantan-1-yl) carbamoyl)benzenesulfonamide (13b), and N-(-Adamantan-1-yl) carbamoyl)-4-methoxybenzenesulfonamide (13c).

General Procedure D for Synthesis of Sulfonylureas: To a solution of appropriate sulfonyl carbamate (1.2 eq) in toluene was added amino component (1 eq) and the resulting slurry was refluxed for 4 h. After cooling to room temperature, the toluene solution was evaporated, and the slurry was triturated with isopropyl alcohol to obtain white slurry. The solution was filtered and washed with a mixture of cold IPA and hexanes (1:1) to give sulfonyl urea compounds as a white solid/powder.

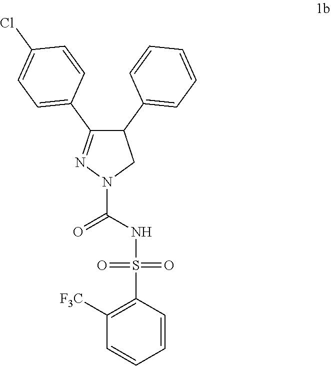 [SMILES: O=C(NS(=O)(=O)c1ccccc1C(F)(F)F)N1CC(c2ccccc2)C(c2ccc(Cl)cc2)=N1]

3-(4-chlorophenyl)-4-phenyl-N-((2-(trifluoromethyl)phenyl) sulfonyl)-4,5-dihydro-1Hpyrazole-1-carboxamide (1b): Using General Procedure D, ethyl ((2-trifluoromethylphenyl) sulfonyl) carbamate (1.2 eq) and 3-(4-chlorophenyl)-4-phenyl-4,5-dihydro-1H-pyrazole (2.56 g, 10.0 mmol) gave 1b as a white solid (3.7 g, 73%). 1H-NMR (800 MHZ, CDCl₃): δ 8.91 (s, 1H), 8.57 (d, J=7.3 Hz, 1H), 7.90 (d, J=6.9 Hz, 1H), 7.78 (t, J=7.5 Hz, 2H), 7.52 (d, J=8.1 Hz, 2H), 7.29 (td, J=17.3, 9.4 Hz, 5H), 7.11 (d, J=7.2 Hz, 2H), 4.71 (dd, J=11.2, 4.9 Hz, 1H), 4.28 (t, J=11.5 Hz, 1H), 3.87 (dd, J=10.8, 4.8 Hz, 1H). 13C-NMR (201 MHZ, CDCl₃): δ 156.6, 147.6, 139.1, 137.4, 136.9, 134.3, 133.9, 132.5, 129.7, 129.2, 128.8, 128.35, 128.29, 128.17, 127.3, 54.1, 51.7. LRMS 452.1.

3-(4-chlorophenyl)-N-((2-fluorophenyl) sulfonyl)-4-phenyl-4,5-dihydro-1H-pyrazole-1-carboxamide (1p): Using General Procedure D, methyl ((2-fluorophenyl) sulfonyl) carbamate (1.3 eq) and 3-(4-chlorophenyl)-4-phenyl-4,5-dihydro-1H-pyrazole (1.0 g, 3.9 mmol) gave 1p as a (720 mg, 40%) white solid. 1H-NMR (800 MHZ, CDCl₃): δ 8.94 (s, 1H), 8.15 (t, J=7.1 Hz, 1H), 7.64 (d, J=4.8 Hz, 1H), 7.55 (d, J=8.2 Hz, 2H), 7.34 (t, J=7.5 Hz, 1H), 7.30 (td, J=16.0, 8.1 Hz, 4H), 7.23 (d, J=9.0 Hz, 2H), 7.12 (d, J=7.3 Hz, 2H), 4.71 (dd, J=11.5, 5.3 Hz, 1H), 4.30 (t, J=11.6 Hz, 1H), 3.89 (dd, J=11.5, 5.4 Hz, 1H). 13C-NMR (201 MHZ, CDCl₃): δ 160.1, 158.7, 156.9, 147.8, 139.2, 136.9, 136.3, 132.2, 129.3, 128.8, 128.2, 127.4, 124.7, 117.3, 117.1, 54.1, 51.7. LRMS 452.1.

R-3-(4-chlorophenyl)-N-((4-chlorophenyl) sulfonyl)-4-phenyl-4,5-dihydro-1H-pyrazole-1-carboxamide (1r): 1H-NMR (800 MHZ; CDCl₃): δ 8.09 (d, J=7.4 Hz, 2H), 7.52 (d, J=7.9 Hz, 4H), 7.29 (d, J=6.9 Hz, 2H), 7.26 (d, J=7.3 Hz, 3H), 7.10 (d, J=7.4 Hz, 2H), 4.70-4.69 (m, 1H), 4.30-4.28 (m, 1H), 3.89-3.88 (m, 1H) (known parent racemic compound).

S-3-(4-chlorophenyl)-N-((4-chlorophenyl) sulfonyl)-4-phenyl-4,5-dihydro-1H-pyrazole-1-carboxamide (1s): 1H-NMR (800 MHZ; CDCl₃): δ 8.10 (d, J=8.4 Hz, 2H), 7.53 (d, J=8.4 Hz, 5H), 7.30 (t, J=7.4 Hz, 6H), 7.11 (d, J=7.5 Hz, 2H), 4.71 (dd, J=11.5, 5.4 Hz, 1H), 4.31 (t, J=11.6 Hz, 1H), 3.90 (dt, J=14.2, 3.9 Hz, 1H) (known parent racemic compound).

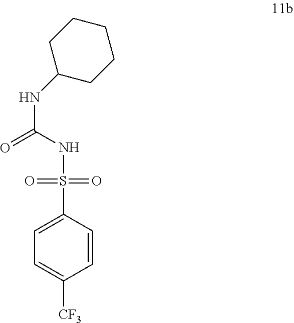 [SMILES: O=C(NC1CCCCC1)NS(=O)(=O)c1ccc(C(F)(F)F)cc1]

N-(Cyclohexylcarbamoyl)-4-(trifluoromethyl)benzenesulfonamide (11b): Utilizing General procedure D, methyl ((4-trifluormethyl) sulfonyl) carbamate (2.9 g 10.3 mmol) and cyclohexylamine (853 mg, 8.8 mmol) compound 15 was obtained as a white solid (1.5 g, 51%). 1H NMR (800 MHZ; CDCl₃): δ 8.03 (d, J=8.1 Hz, 3H), 7.82 (d, J=8.3 Hz, 2H), 6.41 (s, 1H), 3.61 (s, 1H), 1.86-1.85 (m, 2H), 1.69 (t, J=0.3 Hz, 2H), 1.61-1.57 (m, 5H), 1.34 (s, 2H), 1.22-1.20 (m, 3H). ¹³C NMR (201 MHz; CDCl₃): δ 150.7, 143.2, 135.5, 127.7, 126.6, 49.4, 33.0, 25.4, 24.6; LRMS 351.1, HRMS (C₁₄H₁₈N₂O₃SF₃) [M+H]⁺ found m/z 351.0987, calcd 351.0990.

### Example 4: Synthesis and Characterization of Sulfonyl Thioureas

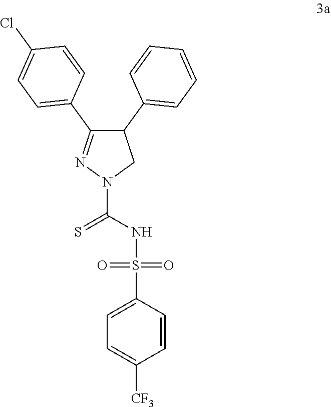 [SMILES: O=S(=O)(NC(=S)N1CC(c2ccccc2)C(c2ccc(Cl)cc2)=N1)c1ccc(C(F)(F)F)cc1]

3-(4-chlorophenyl)-4-phenyl-N-((4-(trifluoromethyl)phenyl) sulfonyl)-4,5-dihydro-1H-pyrazole-1-carbothioamide (3a): General Procedure A: To a mixture of compound 1a (100 mg, 0.2 mmol) in toluene (5 mL), POCl₃ (0.03 mL, 1.5 mmol), was added, followed by the addition of N,N-diisopropylethylamine (DIPEA) (0.03 mL, 1.5 mmol) and the mixture was refluxed for 1.5 h under N₂ atmosphere. The reaction mixture was then cooled, and the excess reagents in toluene were evaporated in vacuo. The imidoyl chloride intermediate was dissolved in methanol (10 mL), to this solution was added dropwise, sodium thiosulfate (2 eq, 63 mg, 0.4 mmol) dissolved in 0.5 mL water and the reaction was heated to 90° C. Upon completion of reaction as seen by TLC, the reaction mixture was cooled to room temperature and methanol was evaporated. The organic mixture was the extracted into dichloromethane, washed with brine, and dried over Na₂SO₄. The sticky solid was purified by flash chromatography (40% hexanes in EtOAc) to compound 3a as a pale white powder (94 mg, 91% yield). 1H-NMR (800 MHZ, CDCl₃): δ 9.62 (s, 1H), 8.30 (d, J=7.6 Hz, 2H), 7.81 (d, J=7.8 Hz, 2H), 7.60-7.59 (m, 2H), 7.32-7.28 (m, 5H), 7.11-7.10 (m, 2H), 4.78-4.74 (m, 1H), 4.60-4.56 (m, 1H), 4.20-4.17 (m, 1H). 13C-NMR (201 MHZ, CDCl₃): δ 169.3, 159.5, 142.2, 138.8, 137.8, 135.49, 135.35, 130.1, 129.8, 129.41, 129.30, 128.5, 127.7, 127.4, 125.9, 58.1, 51.4. LRMS 524.1, HRMS (C₂₃H₁₈ClN₃O₂F₃S₂) [M+H]⁺ found m/z 524.0480, calcd 524.0481.

Gram Scale Synthesis of 3a: To a mixture of compound 1a (2.02 g, 4.00 mmol) in toluene (20 mL), POCl₃ (0.56 mL, 6 mmol), was added, followed by the addition of N,N-diisopropylethylamine (DIPEA) (1.05 mL, 6 mmol) and the mixture was refluxed for 2 h under N₂ atmosphere. The reaction mixture was then cooled, and the excess reagents in toluene were chased off in vacuo. The pale yellow imidoyl chloride intermediate (LCMS showing no traces of urea) was dissolved in methanol (20 mL), to this solution was added dropwise, sodium thiosulfate (2 eq, 1.3 g, 8 mmol) dissolved in 2 mL water and the reaction was heated to 90° C. Upon completion of the reaction as seen by TLC, the reaction mixture was cooled to room temperature and methanol was evaporated. The organic mixture was then extracted into dichloromethane (30 mL) washed with brine and dried over Na₂SO₄. The solvent removed in vacuo and dried thoroughly to afford 3a as a yellow sticky solid (up to 90% pure). The sticky solid was run through a filter silica column and washed with 50% hexanes/ethyl acetate to give compound 3a as an off-white powder (1.49 g, 71% yield).

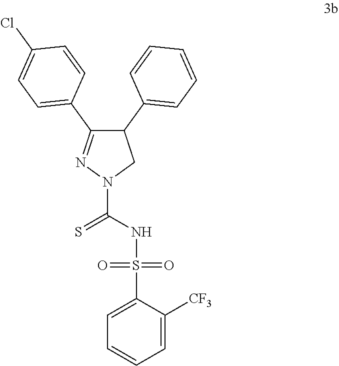 [SMILES: O=S(=O)(NC(=S)N1CC(c2ccccc2)C(c2ccc(Cl)cc2)=N1)c1ccccc1C(F)(F)F]

3-(4-Chlorophenyl)-4-phenyl-N-((2-(trifluoromethyl)phenyl) sulfonyl)-4,5-dihydro-1Hpyrazole-1-carbothioamide (3b): Following General procedure A, Compound (urea) 1b (220 mg, 0.43 mmol) was converted in a two-step, one-pot protocol to title compound thiourea 3b (184 mg, 88% yield) as a pale white solid. ¹H NMR (800 MHZ, CDCl₃): δ 9.78 (d, J=0.5 Hz, 1H), 8.63 (d, J=7.6 Hz, 1H), 7.88 (d, J=7.3 Hz, 1H), 7.77 (q, J=7.9 Hz, 2H), 7.58 (d, J=8.4 Hz, 2H), 7.33-7.30 (m, 4H), 7.27 (d, J=7.3 Hz, 1H), 7.10 (d, J=7.7 Hz, 2H), 4.74 (dd, J=11.3, 5.4 Hz, 1H), 4.56 (t, J=12.0 Hz, 1H), 4.19 (dd, J=12.7, 5.3 Hz, 1H). ¹³C NMR (201 MHZ, CDCl₃): δ 169.1, 159.3, 138.9, 137.7, 136.8, 135.9, 133.9, 131.9, 129.8, 129.4, 129.3, 128.5, 127.4, 58.0, 51.4. LRMS 524.1, HRMS (C₂₃H₁₈ClN₃O₂F₃S₂) [M+H]⁺ found m/z 524.0480, calcd 524.0481.

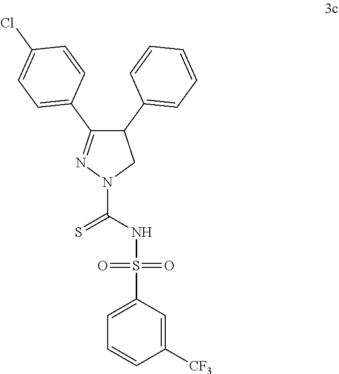 [SMILES: O=S(=O)(NC(=S)N1CC(c2ccccc2)C(c2ccc(Cl)cc2)=N1)c1cccc(C(F)(F)F)c1]

3-(4-Chlorophenyl)-4-phenyl-N-((3-(trifluoromethyl)phenyl) sulfonyl)-4,5-dihydro-1Hpyrazole-1-carbothioamide (3c): Following General procedure A, Compound (urea) 1c (136 mg, 0.27 mmol) was converted in a two-step, one-pot protocol to title compound thiourea 3c (110 mg, 78% yield) as an off-white solid. ¹H NMR (800 MHZ, CDCl₃): δ 9.62 (s, 1H), 8.42-8.40 (m, 2H), 7.89 (d, J=7.8 Hz, 1H), 7.70 (t, J=7.8 Hz, 1H), 7.60 (d, J=8.6 Hz, 2H), 7.33-7.27 (m, 5H), 7.11 (d, J=7.4 Hz, 2H), 4.76 (dd, J=11.4, 5.6 Hz, 1H), 4.59 (t, J=12.1 Hz, 1H), 4.20 (dd, J=12.8, 5.6 Hz, 1H). ¹³C NMR (201 MHz, CDCl₃): δ 169.3, 159.4, 139.9, 138.9, 137.8, 133.1, 130.5, 129.9, 129.4, 129.3, 128.5, 127.7, 127.4, 126.6, 58.1, 51.4. LRMS 524.1, HRMS (C₂₃H₁₈ClN₃O₂F₃S₂) [M+H]⁺ found m/z 524.0480, calcd 524.0481.

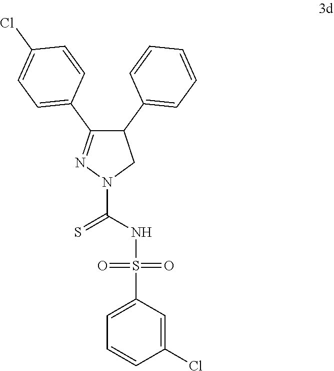 [SMILES: O=S(=O)(NC(=S)N1CC(c2ccccc2)C(c2ccc(Cl)cc2)=N1)c1cccc(Cl)c1]

3-(4-Chlorophenyl)-N-((3-chlorophenyl) sulfonyl)-4-phenyl-4,5-dihydro-1H-pyrazole-1-carbothioamide (3d): Following General procedure A, Compound (urea) 1d (104 mg, 0.22 mmol) was converted in a two-step, one-pot protocol to title compound thiourea 3d (78 mg, 73% yield) as a pale solid. ¹H NMR (800 MHZ, CDCl₃): δ 9.62 (s, 1H), 8.14 (d, J=1.2 Hz, 1H), 8.08 (d, J=7.9 Hz, 1H), 7.60-7.59 (m, 3H), 7.49 (s, 1H), 7.30 (td, J=18.5, 8.1 Hz, 5H), 7.12 (d, J=7.5 Hz, 2H), 4.76 (dd, J=11.1, 5.1 Hz, 1H), 4.61-4.58 (m, 1H), 4.21-4.19 (m, 1H). ¹³C NMR (201 MHZ, CDCl₃): 169.3, 159.3, 140.2, 138.9, 137.7, 134.9, 134.0, 129.95, 129.81, 129.3, 128.4, 127.84, 127.73, 127.4, 58.1, 51.3 LRMS 490.1, HRMS (C₂₂H₁₈Cl₂N₃O₂S₂) [M+H]⁺ found m/z 490.0222, calcd 490.0217.

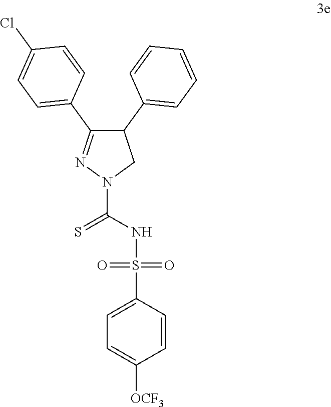 [SMILES: O=S(=O)(NC(=S)N1CC(c2ccccc2)C(c2ccc(Cl)cc2)=N1)c1ccc(OC(F)(F)F)cc1]

3-(4-Chlorophenyl)-4-phenyl-N-((4-(trifluoromethoxy)phenyl) sulfonyl)-4,5-dihydro-1Hpyrazole-1-carbothioamide (3e): Following General procedure A, Compound (urea) 1e (300 mg, 0.57 mmol) was converted in a two-step, one-pot protocol to title compound thiourea 3e (257 mg, 83% yield) as a pale solid. ¹H NMR (800 MHZ, CDCl₃): δ 9.60 (s, 1H), 8.23 (d, J=8.2 Hz, 2H), 7.59 (d, J=8.0 Hz, 2H), 7.35 (d, J=8.3 Hz, 2H), 7.30 (q, J=9.1 Hz, 5H), 7.11 (d, J=7.4 Hz, 2H), 4.75 (dd, J=11.2, 5.4 Hz, 1H), 4.59 (t, J=12.1 Hz, 1H), 4.20 (dd, J=12.6, 5.3 Hz, 1H). ¹³C NMR (201 MHz, CDCl₃): δ 169.5, 159.3, 153.1, 138.9, 137.8, 136.8, 131.9, 129.9, 129.4, 129.3, 128.5, 127.4, 120.32, 58.1, 51.3. LRMS 540.1, HRMS (C₂₃H₁₈ClN₃O₃F₃S₂) [M+H]⁺ found m/z 540.0425, calcd 540.0430.

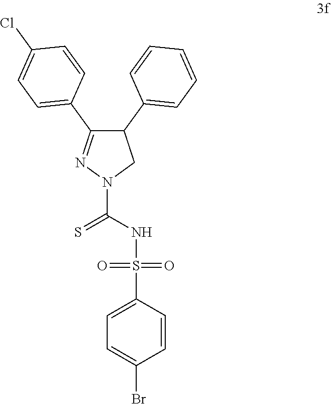 [SMILES: O=S(=O)(NC(=S)N1CC(c2ccccc2)C(c2ccc(Cl)cc2)=N1)c1ccc(Br)cc1]

N-((4-Bromophenyl) sulfonyl)-3-(4-chlorophenyl)-4-phenyl-4,5-dihydro-1H-pyrazole-1-carbothioamide (3f): Following General procedure A, Compound (urea) 1f (96 mg, 0.18 mmol) was converted in a two-step, one-pot protocol to title compound thiourea 3f (62 mg, 52% yield) as a pale white solid. ¹H NMR (800 MHz; CDCl₃): δ 9.59 (s, 1H), 8.04 (s, 2H), 7.68 (d, J=8.3 Hz, 2H), 7.59 (d, J=8.3 Hz, 2H), 7.31 (dt, J=16.8, 9.0 Hz, 6H), 7.11 (d, J=7.4 Hz, 2H), 4.76 (t, J=5.7 Hz, 1H), 4.59 (t, J=12.0 Hz, 1H), 4.20 (dd, J=12.7, 5.4 Hz, 1H). ¹³C NMR (201 MHz, CDCl₃): δ 159.3, 154.8, 138.9, 137.8, 132.1, 131.1, 130.3, 130.0, 129.9, 129.3, 129.2, 128.5, 127.8, 127.4, 126.55, 118.9, 58.1, 51.3. LRMS 534.0, HRMS (C₂₂H₁₈ClBrN₃O₂S₂) [M+H]⁺ found m/z 533.9718, calcd 533.9712.

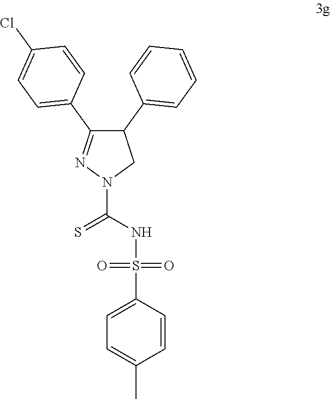 [SMILES: Cc1ccc(S(=O)(=O)NC(=S)N2CC(c3ccccc3)C(c3ccc(Cl)cc3)=N2)cc1]

3-(4-Chlorophenyl)-4-phenyl-N-tosyl-4,5-dihydro-1H-pyrazole-1-carbothioamide (3g): Following General procedure A, Compound (urea) 1g (240 mg, 0.53 mmol) was converted in a two-step, one-pot protocol to title compound thiourea 3g (109 mg, 56% yield) as a pale solid. 1H NMR (800 MHZ, CDCl₃): δ 9.59 (s, 1H), 8.05 (d, J=8.1 Hz, 2H), 7.59 (d, J=8.3 Hz, 2H), 7.34 (d, J=8.0 Hz, 2H), 7.31 (d, J=5.6 Hz, 5H), 7.10 (d, J=7.5 Hz, 2H), 4.74 (dd, J=11.4, 5.5 Hz, 1H), 4.58 (t, J=12.1 Hz, 1H), 4.20 (dd, J=12.6, 5.5 Hz, 1H), 2.44 (s, 3H). ¹³C NMR (201 MHz, CDCl₃): δ 169.8, 158.9, 145.0, 139.0, 137.6, 135.7, 129.8, 129.6, 129.33, 129.26, 128.4, 127.9, 127.4, 58.1, 51.2, 21.9. LRMS 470.1, HRMS (C₂₃H₂₁ClN₃O₂S₂) [M+H]⁺ found m/z 470.0769, calcd 470.0764.

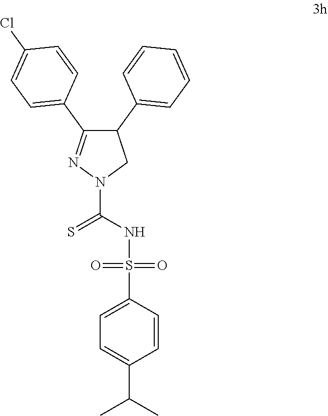 [SMILES: CC(C)c1ccc(S(=O)(=O)NC(=S)N2CC(c3ccccc3)C(c3ccc(Cl)cc3)=N2)cc1]

3-(4-chlorophenyl)-N-((4-isopropylphenyl) sulfonyl)-4-phenyl-4,5-dihydro-1H-pyrazole-1-carbothioamide (3h): Following General procedure A, Compound (urea) 1h (300 mg, 0.62 mmol) was converted in a two-step, one-pot protocol to title compound thiourea 3h (191 mg, 62% yield) as a pale solid. 1H-NMR (800 MHZ; CDCl₃): δ 9.61 (s, 1H), 8.08 (d, J=7.8 Hz, 2H), 7.59 (d, J=8.0 Hz, 2H), 7.39 (d, J=7.8 Hz, 2H), 7.30 (t, J=8.6 Hz, 4H), 7.11 (d, J=7.2 Hz, 2H), 4.74 (dd, J=11.2, 5.2 Hz, 1H), 4.59 (t, J=12.1 Hz, 1H), 4.20 (dd, J=12.5, 5.1 Hz, 1H), 2.99 (t, J=6.7 Hz, 1H), 1.28 (d, J=6.7 Hz, 6H). ¹³C NMR (201 MHz, CDCl₃): δ 169.8, 158.9, 155.5, 138.9, 137.5, 135.9, 129.8, 129.6, 129.2, 128.4, 127.9, 127.4, 126.9, 58.1, 51.2, 34.4, 23.7. LRMS 498.1, HRMS (C₂₅H₂₅ClN₃O₂S₂) [M+H]⁺ found m/z 498.1083, calcd 498.1077.

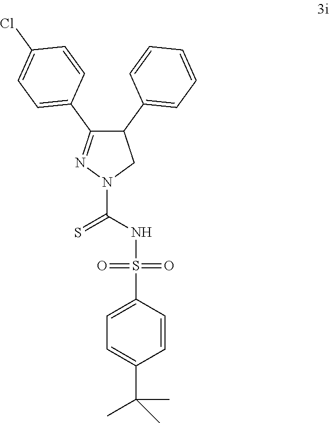 [SMILES: CC(C)(C)c1ccc(S(=O)(=O)NC(=S)N2CC(c3ccccc3)C(c3ccc(Cl)cc3)=N2)cc1]

N-((4-(tert-butyl)phenyl) sulfonyl)-3-(4-chlorophenyl)-4-phenyl-4,5-dihydro-1H-pyrazole-1-carbothioamide (3i): Following General procedure A, Compound (urea) 1i (100 mg, 0.20 mmol) was converted in a two-step, one-pot protocol to title compound thiourea 3i (52 mg, 50% yield) as a pale solid. 1H-NMR (800 MHZ; CDCl₃): δ 9.61 (s, 1H), 8.09 (d, J=8.4 Hz, 2H), 7.59 (d, J=8.5 Hz, 2H), 7.55 (d, J=8.4 Hz, 2H), 7.30 (t, J=8.5 Hz, 4H), 7.27 (s, 1H), 7.11 (d, J=7.7 Hz, 2H), 4.74 (dd, J=11.4, 5.5 Hz, 1H), 4.59 (t, J=12.1 Hz, 1H), 4.21 (dd, J=12.6, 5.5 Hz, 1H), 1.35 (s, 9H). ¹³C NMR (201 MHz, CDCl₃): δ 169.8, 158.8, 157.8, 139.0, 137.5, 135.6, 129.8, 129.2, 127.4, 125.8, 58.1, 51.2, 51.1, 31.2. LRMS 512.1, HRMS (C₂₆H₂₇ClN₃O₂S₂) [M+H]⁺ found m/z 512.1234, calcd 512.1233.

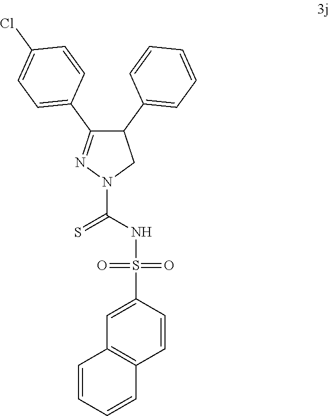 [SMILES: O=S(=O)(NC(=S)N1CC(c2ccccc2)C(c2ccc(Cl)cc2)=N1)c1ccc2ccccc2c1]

3-(4-chlorophenyl)-N-(naphthalen-2-ylsulfonyl)-4-phenyl-4,5-dihydro-1H-pyrazole-1-carbothioamide (3j): Following General procedure A, Compound (urea) 1j (90 mg, 0.18 mmol) was converted in a two-step, one-pot protocol to title compound thiourea 3j (72 mg, 77% yield) as a pale solid. 1H-NMR (800 MHZ; CDCl₃): δ 9.60 (s, 1H), 8.11 (d, J=7.2 Hz, 2H), 7.60-7.59 (m, 2H), 7.51 (d, J=7.6 Hz, 2H), 7.31 (t, J=8.2 Hz, 4H), 7.28 (d, J=7.6 Hz, 1H), 7.11 (d, J=7.3 Hz, 2H), 4.75 (dd, J=11.3, 5.4 Hz, 1H), 4.59 (d, J=11.9 Hz, 1H), 4.19 (dd, J=12.6, 5.3 Hz, 1H). ¹³C NMR (201 MHz, CDCl₃): δ 169.7, 159.0, 145.98, 138.9, 132.0, 131.9, 129.8, 129.5, 129.4, 129.3, 128.9, 128.4, 128.1, 127.7, 127.4, 123.7, 58.1, 51.3. LRMS 506.1, HRMS (C₂₆H₂₁ClN₃O₃S₂) [M+H]⁺ found m/z 506.0769, calcd 506.0764.

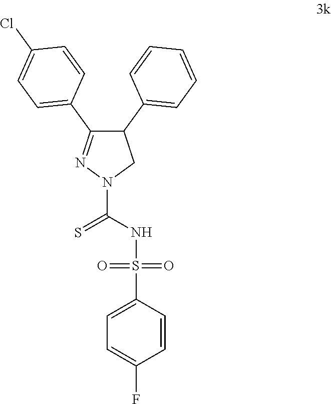 [SMILES: O=S(=O)(NC(=S)N1CC(c2ccccc2)C(c2ccc(Cl)cc2)=N1)c1ccc(F)cc1]

3-(4-Chlorophenyl)-N-((4-fluorophenyl) sulfonyl)-4-phenyl-4,5-dihydro-1H-pyrazole-1-carbothioamide (3k): Following General procedure A, Compound (urea) 1k (32 mg, 0.07 mmol) was converted in a two-step, one-pot protocol to title compound thiourea 3k (15 mg, 45% yield) as an off-white solid. ¹H NMR (800 MHZ, CDCl₃): δ 9.58 (s, 1H), 8.21-8.19 (m, 2H), 7.60-7.59 (m, 2H), 7.33-7.29 (m, 4H), 7.28 (d, J=7.4 Hz, 1H), 7.21 (t, J=8.6 Hz, 2H), 7.11 (d, J=7.2 Hz, 2H), 4.75 (dd, J=11.5, 5.6 Hz, 1H), 4.59 (t, J=12.1 Hz, 1H), 4.20 (dd, J=12.7, 5.6 Hz, 1H). ¹³C NMR (201 MHz, CDCl₃): δ 169.6, 159.2, 138.9, 137.7, 132.6, 129.8, 129.37, 129.26, 128.5, 127.4, 116.10, 115.96, 58.1, 51.3. LRMS 474.1, HRMS (C₂₂H₁₈ClFN₃O₂S₂) [M+H]⁺ found m/z 474.0520, calcd 474.0513.

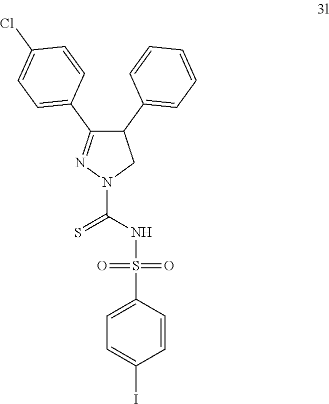 [SMILES: O=S(=O)(NC(=S)N1CC(c2ccccc2)C(c2ccc(Cl)cc2)=N1)c1ccc(I)cc1]

3-(4-Chlorophenyl)-N-((4-iodophenyl) sulfonyl)-4-phenyl-4,5-dihydro-1H-pyrazole-1-carbothioamide (31): Following General procedure A, Compound (urea) 11 (85 mg, 0.15 mmol) was converted in a two-step, one-pot protocol to title compound thiourea 31 (48 mg, 55% yield) as a pale solid. ¹H NMR (800 MHZ, CDCl₃): δ 9.59 (s, 1H), 7.91-7.87 (m, 4H), 7.59 (dd, J=8.6, 2.1 Hz, 2H), 7.31 (dd, J=8.5, 7.0 Hz, 4H), 7.28 (s, 1H), 7.11 (d, J=7.5 Hz, 2H), 4.77-4.75 (m, 1H), 4.60-4.57 (m, 1H), 4.20 (ddd, J=12.7, 5.5, 2.0 Hz, 1H). ¹³C NMR (201 MHz, CDCl₃): δ 169.3, 159.2, 138.8, 138.51, 138.37, 138.26, 138.0, 130.8, 129.8, 129.37, 129.26, 128.4, 127.7, 127.4, 58.1, 51.3. LRMS 582.0, HRMS (C₂₂H₁₈ClIN₃O₂S₂) [M+H]⁺ found m/z 581.9570, calcd 581.9574.

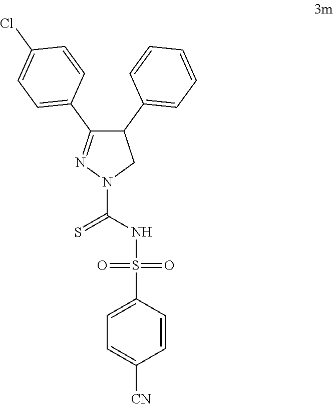 [SMILES: N#Cc1ccc(S(=O)(=O)NC(=S)N2CC(c3ccccc3)C(c3ccc(Cl)cc3)=N2)cc1]

3-(4-Chlorophenyl)-N-((4-cyanophenyl) sulfonyl)-4-phenyl-4,5-dihydro-1H-pyrazole-1-carbothioamide (3m): Following General procedure A, Compound (urea) 1m (1.0 g, 2.2 mmol) was converted in a two-step, one-pot protocol to title compound thiourea 3m (820 mg, 79% yield) as a pale yellow solid. ¹H NMR (800 MHZ, CDCl₃): δ 8.28 (d, J=8.1 Hz, 2H), 7.83 (d, J=8.2 Hz, 2H), 7.60 (d, J=8.5 Hz, 2H), 7.32 (dd, J=13.6, 7.7 Hz, 5H), 7.28 (d, J=7.3 Hz, 1H), 7.11-7.10 (m, 2H), 4.77 (dd, J=11.3, 5.4 Hz, 1H), 4.58 (t, J=12.1 Hz, 1H), 4.19 (dd, J=12.7, 5.6 Hz, 1H). ¹³C NMR (201 MHz, CDCl₃): δ 169.0, 159.7, 142.7, 138.7, 132.5, 130.2, 129.8, 129.41, 129.30, 128.5, 127.4, 117.4, 58.1, 51.3. LRMS 481.1, HRMS (C₂₃H₁₈ClN₄O₂S₂) [M+H]⁺ found m/z 481.0557, calcd 481.0560.

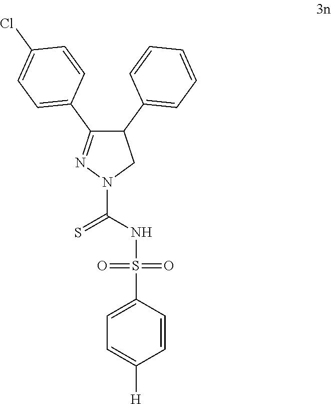 [SMILES: O=S(=O)(NC(=S)N1CC(c2ccccc2)C(c2ccc(Cl)cc2)=N1)c1ccccc1]

3-(4-Chlorophenyl)-N-(phenyl) sulfonyl)-4-phenyl-4,5-dihydro-1H-pyrazole-1-carbothioamide (3n): Following General procedure A, Compound (urea) 1n (150 mg, 0.34 mmol) was converted in a two-step, one-pot protocol to title compound thiourea 3n (58 mg, 37% yield) as a pale solid. ¹H NMR (800 MHZ, CDCl₃): δ 8.18-8.17 (m, 2H), 7.65-7.63 (m, 1H), 7.59 (d, J=8.1 Hz, 2H), 7.56-7.54 (m, 2H), 7.32-7.28 (m, 5H), 7.11-7.10 (m, 2H), 4.75-4.73 (m, 1H), 4.60-4.57 (m, 1H), 4.21-4.19 (m, 1H). ¹³C NMR (201 MHz, CDCl₃): δ 169.7, 159.0, 139.0, 138.7, 133.9, 129.8, 129.48, 129.36, 129.26, 128.8, 128.4, 127.4, 58.1, 51.3. LRMS 456.1, HRMS (C₂₂H₁₉ClN₃O₂S₂) [M+H]⁺ found m/z 456.0609, calcd 456.0607.

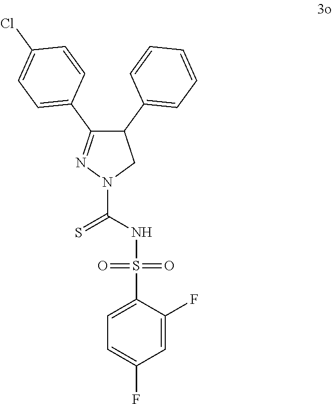 [SMILES: O=S(=O)(NC(=S)N1CC(c2ccccc2)C(c2ccc(Cl)cc2)=N1)c1ccc(F)cc1F]

3-(4-Chlorophenyl)-N-((2,4-Difluorophenyl) sulfonyl)-4-phenyl-4,5-dihydro-1H-pyrazole-1-carbothioamide (30): Following General procedure A, Compound (urea) 10 (200 mg, 0.42 mmol) was converted in a two-step, one-pot protocol to title compound thiourea 30 (82 mg, 40% yield) as a pale solid. Following General procedure B, Compound (urea) 10 (100 mg, 0.21 mmol) was converted in a two-step, one-pot protocol to title compound thiourea 30 (78 mg, 76% yield) as a pale solid. ¹H NMR (800 MHZ, CDCl₃): δ 8.20 (q, J=7.3 Hz, 1H), 7.61 (d, J=8.6 Hz, 2H), 7.32 (dd, J=16.8, 8.0 Hz, 5H), 7.28 (d, J=7.4 Hz, 2H), 7.12 (d, J=7.1 Hz, 2H), 7.07-7.05 (m, 1H), 6.96-6.94 (m, 1H), 4.77 (dd, J=11.4, 5.5 Hz, 1H), 4.59 (dd, J=12.5, 11.6 Hz, 1H), 4.20 (dd, J=12.7, 5.5 Hz, 1H). ¹³C NMR (201 MHZ, CDCl₃): δ 169.2, 159.5, 135.6, 129.8, 129.35, 129.28, 128.5, 127.7, 127.4, 111.92, 111.81, 105.6, 58.0, 51.3. LRMS 492.1, HRMS (C₂₂H₁₇ClN₃O₂F₂S₂) [M+H]⁺ found m/z 492.0415, calcd 492.0419.

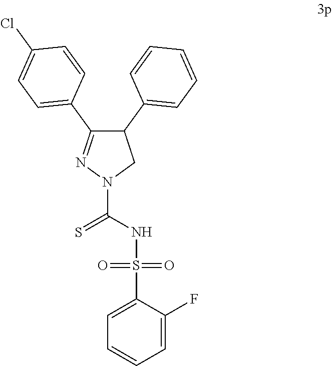 [SMILES: O=S(=O)(NC(=S)N1CC(c2ccccc2)C(c2ccc(Cl)cc2)=N1)c1ccccc1F]

3-(4-Chlorophenyl)-N-((2-fluorophenyl) sulfonyl)-4-phenyl-4,5-dihydro-1H-pyrazole-1-carbothioamide (3p): Following General procedure A, Compound (urea) 1p (50 mg, 0.11 mmol) was converted in a two-step, one-pot protocol to title compound thiourea 3p (16 mg, 31% yield) as a pale solid. ¹H NMR (800 MHz, CDCl₃): δ 8.19 (t, J=7.3 Hz, 1H), 7.65-7.62 (m, 3H), 7.34 (dd, J=15.9, 8.1 Hz, 8H), 7.14 (d, J=7.6 Hz, 2H), 4.78-4.76 (m, 1H), 4.61 (d, J=12.5 Hz, 1H), 4.22 (dd, J=12.3, 5.1 Hz, 1H). ¹³C NMR (201 MHz, CDCl₃): δ 169.4, 158.5, 138.9, 137.7, 136.2, 133.6, 129.8, 129.4, 128.5, 128.1, 127.8, 127.4, 124.3, 120.3, 117.01, 116.95, 58.1, 51.3. LRMS 474.1, HRMS (C₂₂H₁₈ClFN₃O₂S₂) [M+H]⁺ found m/z 474.0520, calcd 474.0513.

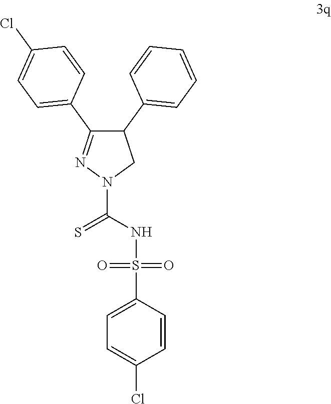 [SMILES: O=S(=O)(NC(=S)N1CC(c2ccccc2)C(c2ccc(Cl)cc2)=N1)c1ccc(Cl)cc1]

3-(4-Chlorophenyl)-N-((4-chlorophenyl) sulfonyl)-4-phenyl-4,5-dihydro-1H-pyrazole-1-carbothioamide (3g): Following General procedure A, Compound (urea) 1q (500 mg, 1.05 mmol) was converted in a two-step, one-pot protocol to title compound thiourea 3q (385 mg, 75% yield) as a pale white solid. ¹H NMR (800 MHZ; CDCl₃): δ 9.55 (s, 1H), 8.06-8.05 (m, 2H), 7.54 (d, J=8.0 Hz, 2H), 7.46-7.45 (m, 2H), 7.26-7.24 (m, 3H), 7.21-7.20 (m, 2H), 7.05 (d, J=6.8 Hz, 2H), 4.70 (dq, J=8.6, 2.7 Hz, 1H), 4.53 (t, J=12.1 Hz, 1H), 4.14 (dq, J=9.7, 2.9 Hz, 1H). ¹³C NMR (201 MHz, CDCl₃): δ 169.4, 159.3, 140.6, 138.9, 137.7, 137.0, 131.0, 129.8, 129.4, 129.3, 129.0, 128.4, 127.4, 58.1, 51.3. LRMS 490.1, HRMS (C₂₂H₁₈Cl₂N₃O₂S₂) [M+H]⁺ found m/z 490.0222, calcd 490.0217. Analytical Chiral HPLC(R,R)-Whelk-O® 1 chiral column (250 mm×4.6 mm/5 μm, available from Regis Technologies, Inc., Morton Grove, IL): 40% hexanes: 60% CH₂Cl₂ (254 nm) Peak 1=4.2 min. Peak 2=5.5 min.

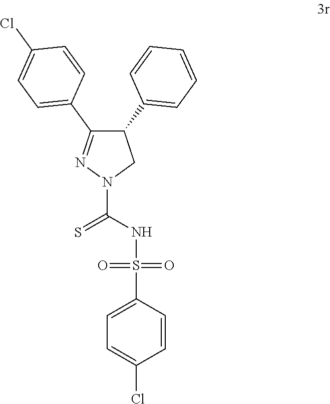 [SMILES: O=S(=O)(NC(=S)N1C[C@@H](c2ccccc2)C(c2ccc(Cl)cc2)=N1)c1ccc(Cl)cc1]

R-3-(4-Chlorophenyl)-N-((4-chlorophenyl) sulfonyl)-4-phenyl-4,5-dihydro-1H-pyrazole-1-carbothioamide (3r): Following General procedure A, Compound (urea) 1r (50 mg, 0.10 mmol) was converted in a two-step, one-pot protocol to title compound thiourea 3r (37 mg, 71% yield) as a pale white solid. ¹H NMR (800 MHZ, CDCl₃): δ 9.59 (d, J=0.5 Hz, 1H), 8.11 (d, J=7.3 Hz, 2H), 7.60 (t, J=8.5 Hz, 3H), 7.52 (d, J=7.4 Hz, 2H), 7.34-7.29 (m, 5H), 7.13-7.10 (m, 2H), 4.77-4.74 (m, 1H), 4.61-4.57 (m, 1H), 4.21-4.18 (m, 1H). LRMS 490.1, HRMS (C₂₂H₁₈Cl₂N₃O₂S₂) [M+H]⁺ found m/z 490.0222, calcd 490.0217. Analytical Chiral HPLC(R,R)-Whelk-O® 1 chiral column (250 mm×4.6 mm/5 μm, available from Regis Technologies, Inc., Morton Grove, IL): 40% hexanes: 60% CH₂Cl₂ (254 nm) Peak 1=NA. Peak 2=5.7 min (ee>99.99%).

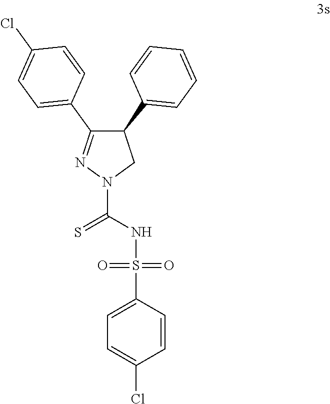 [SMILES: O=S(=O)(NC(=S)N1C[C@H](c2ccccc2)C(c2ccc(Cl)cc2)=N1)c1ccc(Cl)cc1]

S-3-(4-Chlorophenyl)-N-((4-chlorophenyl) sulfonyl)-4-phenyl-4,5-dihydro-1H-pyrazole-1-carbothioamide (3s): Following General procedure A, Compound (urea) 1s (60 mg, 0.13 mmol) was converted in a two-step, one-pot protocol to title compound thiourea 3s (43 mg, 69% yield) as a pale solid. ¹H NMR (800 MHZ, CDCl₃): δ 8.11 (dd, J=8.9, 2.4 Hz, 2H), 7.60-7.58 (m, 2H), 7.51 (dd, J=8.9, 2.3 Hz, 2H), 7.32-7.30 (m, 6H), 7.11-7.10 (m, 2H), 4.75-4.74 (m, 1H), 4.59-4.58 (m, 1H), 4.20 (d, J=12.7 Hz, 1H). LRMS 490.1, HRMS (C₂₂H₁₈Cl₂N₃O₂S₂) [M+H]⁺ found m/z 490.0222, calcd 490.0217. Analytical Chiral HPLC(R,R)-Whelk-O® 1 chiral column (250 mm×4.6 mm/5 μm, available from Regis Technologies, Inc., Morton Grove, IL): 40% hexanes: 60% CH₂Cl₂ (254 nm) Peak 1=4.3 min. Peak 2=5.6 min (ee>97%).

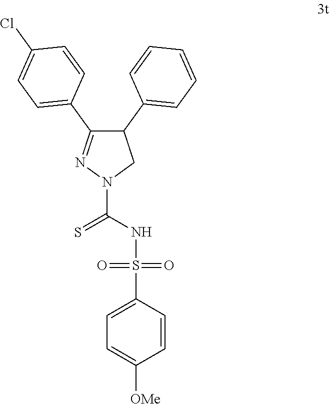 [SMILES: COc1ccc(S(=O)(=O)NC(=S)N2CC(c3ccccc3)C(c3ccc(Cl)cc3)=N2)cc1]

3-(4-Chlorophenyl)-N-((4-methoxyphenyl) sulfonyl)-4-phenyl-4,5-dihydro-1H-pyrazole-1-carbothioamide (3t): Following General procedure A, Compound (urea) 1t (110 mg, 0.23 mmol) was converted in a two-step, one-pot protocol to title compound thiourea 3t (40 mg, 35% yield) as a pale solid. Following General procedure B, Compound (urea) 1t (50 mg, 0.11 mmol) was converted in a two-step, one-pot protocol to title compound thiourea 3t (41 mg, 79% yield) as a white solid. ¹H NMR (800 MHZ, CDCl₃): δ 8.11-8.10 (m, 2H), 7.59-7.58 (m, 2H), 7.32-7.29 (m, 5H), 7.10 (d, J=7.7 Hz, 2H), 7.00 (dd, J=7.2, 1.3 Hz, 2H), 4.74-4.72 (m, 1H), 4.60-4.57 (m, 1H), 4.22-4.19 (m, 1H), 3.90-3.87 (m, 3H). ¹³C NMR (201 MHZ, CDCl₃): δ 169.8, 163.9, 158.8, 139.0, 137.5, 131.9, 129.91, 129.76, 129.2, 128.4, 127.9, 127.4, 113.9, 58.1, 55.8, 51.1. LRMS 486.1, HRMS (C₂₃H₂₁ClN₃O₂S₂) [M+H]⁺ found m/z 486.0710, calcd 486.0713.

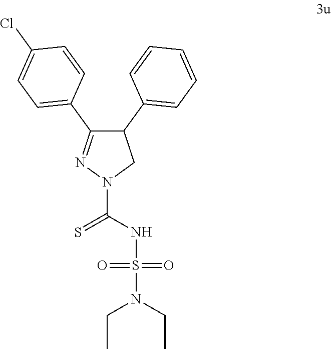 [SMILES: CCN(CC)S(=O)(=O)NC(=S)N1CC(c2ccccc2)C(c2ccc(Cl)cc2)=N1]

3-(4-Chlorophenyl)-N—(N,N-diethylsulfamoyl)-4-phenyl-4,5-dihydro-1H-pyrazole-1-carbothioamide (3u): Following General procedure B, Compound (urea) 1u (63 mg, 0.15 mmol) was converted in a two-step, one-pot protocol to title compound thiourea 3u (47_mg, 72% yield) as a pale solid. ¹H NMR (800 MHZ, CDCl₃): δ 9.43 (s, 1H), 7.58-7.57 (m, 2H), 7.33 (t, J=7.5 Hz, 2H), 7.28 (d, J=8.4 Hz, 3H), 7.13 (d, J=8.0 Hz, 2H), 4.75 (s, 1H), 4.67 (s, 1H), 4.29 (dd, J=12.4, 5.5 Hz, 1H), 3.55 (td, J=15.3, 7.9 Hz, 4H), 1.27 (t, J=7.1 Hz, 6H). ¹³C NMR (201 MHZ, CDCl₃): δ 170.9, 158.5, 139.1, 137.4, 129.8, 129.3, 129.2, 128.4, 128.1, 127.4, 58.2, 51.2, 44.3, 14.3. LRMS 451.1, HRMS (C₂₀H₂₄ClN₄O₂S₂) [M+H]⁺ found m/z 451.1030, calcd 451.1029.

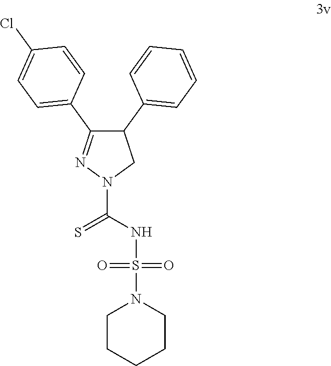 [SMILES: O=S(=O)(NC(=S)N1CC(c2ccccc2)C(c2ccc(Cl)cc2)=N1)N1CCCCC1]

3-(4-Chlorophenyl)-4-phenyl-N-(piperidin-1-ylsulfonyl)-4,5-dihydro-1H-pyrazole-1-carbothioamide (3v): Following General procedure B, Compound (urea) 1v (55 mg, 0.12 mmol) was converted in a two-step, one-pot protocol to title compound thiourea 3v (36 mg, 63% yield) as a pale solid. ¹H NMR (800 MHZ, CDCl₃): δ 9.38 (s, 1H), 7.57 (d, J=8.5 Hz, 2H), 7.33 (t, J=7.3 Hz, 2H), 7.29-7.28 (m, 3H), 7.14 (d, J=7.8 Hz, 2H), 4.77-4.75 (m, 1H), 4.68 (t, J=12.0 Hz, 1H), 4.31-4.28 (m, 1H), 3.53 (s, 4H), 1.69 (d, J=2.9 Hz, 4H), 1.61-1.59 (m, 2H). ¹³C NMR (201 MHz, CDCl₃): 170.9, 158.7, 139.1, 137.4, 129.8, 129.30, 129.15, 128.3, 127.9, 127.4, 58.2, 51.18, 51.07, 48.2, 25.7, 23.9. LRMS 463.1, HRMS (C₂₁H₂₄ClN₄O₂S₂) [M+H]⁺ found m/z 463.1033, calcd 463.1029.

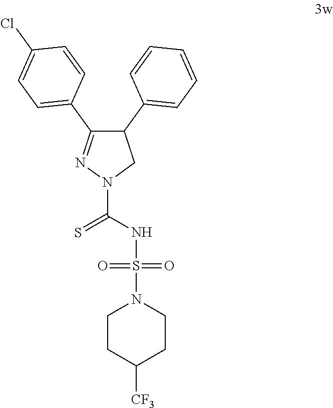 [SMILES: O=S(=O)(NC(=S)N1CC(c2ccccc2)C(c2ccc(Cl)cc2)=N1)N1CCC(C(F)(F)F)CC1]

3-(4-Chlorophenyl)-4-phenyl-N-((4-(trifluoromethyl) piperidin-1-yl) sulfonyl)-4,5-dihydro-1H-pyrazole-1-carbothioamide (3w): Following General procedure B, Compound (urea) 1w (75 mg, 0.15 mmol) was converted in a two-step, one-pot protocol to title compound thiourea 3w (40 mg, 52% yield) as a pale solid. ¹H NMR (800 MHZ, CDCl₃): δ 9.41 (s, 1H), 7.58-7.57 (m, 2H), 7.34-7.30 (m, 3H), 7.29 (d, J=8.4 Hz, 3H), 7.14 (d, J=7.3 Hz, 2H), 4.78 (dd, J=11.4, 5.4 Hz, 1H), 4.67 (t, J=12.0 Hz, 1H), 4.28 (dd, J=12.5, 5.5 Hz, 1H), 4.08 (t, J=15.1 Hz, 2H), 3.16 (dt, J=38.9, 12.7 Hz, 2H), 2.18 (s, 1H), 1.96 (d, J=11.3 Hz, 2H), 1.77-1.74 (m, 2H). ¹³C NMR (201 MHz, CDCl₃): δ 160.2, 138.8, 137.4, 130.2, 129.96, 129.80, 129.2, 128.6, 127.99, 127.84, 127.4, 58.3, 52.0, 46.0, 40.10, 39.96, 39.85, 23.8. LRMS 531.1, HRMS (C₂₂H₂₃ClN₄O₂F₃S₂) [M+H]⁺ found m/z 531.0903, calcd 531.0903.

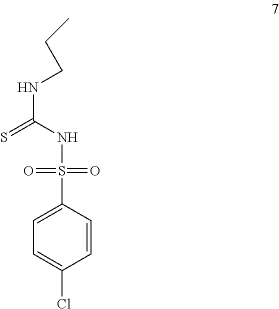 [SMILES: CCCNC(=S)NS(=O)(=O)c1ccc(Cl)cc1]

4-Chloro-N-(propylcarbamothioyl)benzenesulfonamide (7): Following General procedure B, chlorpropamide (1.0 g, 3.6 mmol) was converted in a two-step, one-pot protocol to title compound thiourea 7 (735 mg, 69% yield) as a pale solid. ¹H NMR (800 MHZ; CDCl₃): δ 8.13 (s, 1H), 7.81 (d, J=6.7 Hz, 2H), 7.54-7.53 (m, 2H), 3.54 (d, J=5.4 Hz, 2H), 1.63 (d, J=7.2 Hz, 2H), 0.95-0.93 (m, 3H). ¹³C NMR (201 MHz, CDCl₃): δ 177.9, 141.2, 137.1, 130.1, 128.5, 47.9, 21.8, 11.4. LRMS 293.0, HRMS (C₁₀H₁₄ClN₂O₂S₂) [M+H]⁺ found m/z 293.0190, calcd 293.0185.

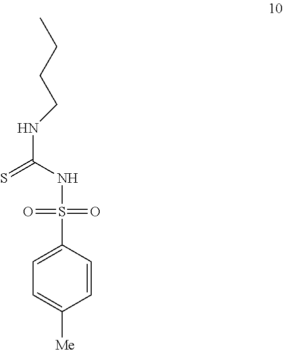 [SMILES: CCCCNC(=S)NS(=O)(=O)c1ccc(C)cc1]

N-(Butylcarbamothioyl)-4-methylbenzenesulfonamide (10): Following General procedure A, Tolbutamide (1.0 g, 3.7 mmol) was converted in a two-step, one-pot protocol to title compound thiourea 10 (695 mg, 66% yield) as a pale solid. ¹H NMR (800 MHZ, CDCl₃): δ 8.11 (s, 1H), 7.75 (d, J=8.3 Hz, 1H), 7.36 (d, J=8.3 Hz, 1H), 3.57-3.57 (m, 1H), 2.45 (s, 2H), 1.57 (t, J=7.4 Hz, 1H), 1.33 (q, J=7.5 Hz, 1H), 0.93 (s, 2H). ¹³C NMR (201 MHz, CDCl₃): δ 178.0, 145.7, 135.7, 130.4, 127.1, 46.0, 30.5, 21.9, 20.1, 13.8. LRMS 287.1, HRMS (C₁₂H₁₉N₂O₂S₂) [M+H]⁺ found m/z 287.0893, calcd 287.0888.

### Example 5: Synthesis and Characterization of Additional Sulfonyl Thioureas

General Procedure C for the Synthesis of Sulfonyl Thioureas: Carboxamide Compound (1 equiv.) was dissolved in dry toluene (8 mL). To the solution was added N,N-diisopropylethylamine (1.5 equiv.) under N₂ atmosphere. POCl₃ (1.5 equiv.) was added to the reaction mixture under ice cold condition, and it was refluxed for 1 hour. Completion of the reaction was confirmed by thin layer chromatography. Toluene was evaporated and intermediate was taken to the next step as is. A solution of sodium thiosulfate (2 equiv.) dissolved in a 9 mL mixture of dioxane and water (8:1) was added dropwise to the reaction mixture. The reaction mixture was heated at 85° C. for 2 hours. The solvent was removed under vacuum, extracted with DCM, and washed with brine solution. The residue was purified by silica gel column chromatography to provide compounds as white solid.

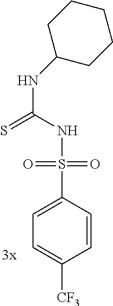 [SMILES: O=S(=O)(NC(=S)NC1CCCCC1)c1ccc(C(F)(F)F)cc1]

N-(Cyclohexylcarbamothioyl)-4-(trifluoromethyl)benzenesulfonamide (3x): Following General procedure A, (100 mg, 0.37 mmol) compound 15 was converted in a two-step, one-pot protocol to title compound thiourea 16 (55 mg, 52% yield) as a pale white solid. ¹H NMR (800 MHz, CDCl₃): δ 8.01 (d, J=8.2 Hz, 4H), 7.85 (d, J=8.2 Hz, 2H), 4.14-4.10 (m, 1H), 1.97-1.96 (m, 2H), 1.71 (dd, J=9.4, 4.0 Hz, 2H), 1.63 (dt, J=8.5, 4.1 Hz, 1H), 1.39 (dd, J=11.0, 2.5 Hz, 2H), 1.28-1.26 (m, 3H). ¹³C NMR (200 MHZ; CDCl₃): δ 176.0, 142.1, 136.2, 127.7, 126.9, 54.9, 31.8, 25.4, 24.5. LRMS 367.1, HRMS (C₁₄H₁₈N₂O₂F₃S₂) [M+H]⁺ found m/z 367.0764, calcd 367.0762.

### Example 6: Synthesis and Characterization of Modified Sulfonyl Thioureas

General Procedure E for the Alkylation of Sulfonyl Thioureas: To a mixture of sulfonylurea compound (e,g 1a) (1 eq) in toluene (8 mL), POCl₃ (1.5 eq), was added, followed by the addition of N,N-diisopropylethylamine (DIPEA) (1.5 eq) and the mixture was heated to 95° C. for 1.5 h under N₂ atmosphere. The reaction mixture was then cooled, and the excess reagents in toluene were evaporated in vacuo. The imidoyl chloride intermediate was dissolved in methanol (10 mL), to this solution was added dropwise, sodium thiosulfate (2 eq, dissolved in 0.5 ml water and the reaction was heated to 90° C. Upon completion of reaction as seen by TLC/LCMS, alkylating agent was added dropwise to the reaction mixture and reaction continued until all the thiourea is consumed as seen by TLC/LCMS. The reaction was cooled to room temperature and methanol was evaporated. The organic mixture was the extracted into dichloromethane, washed with brine, and dried over Na₂SO₄. The solvent was removed in vacuo and dried thoroughly to afford a sticky solid. The sticky solid was purified by flash chromatography (20% hexanes in EtOAc) to give thio-alkylated sulfonyl compounds.

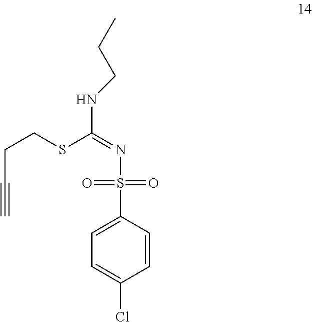 [SMILES: C#CCCS/C(=N\S(=O)(=O)c1ccc(Cl)cc1)NCCC]

But-3-yn-1-yl (Z)—N′-((4-chlorophenyl) sulfonyl)-N-propylcarbamimidothioate (14): Following General procedure E, chlorpropamide (50 mg, 0.18 mmol) was converted in a three-step, one-pot protocol using methyl iodide (2 eq) as the alkylating agent to afford title compound 23 (20 mg, 32% yield) as a pale white solid. ¹H NMR (800 MHZ, CDCl₃): δ 8.19 (s, 1H), 7.84 (d, J=7.9 Hz, 2H), 7.45 (d, J=8.1 Hz, 2H), 3.26 (t, J=5.4 Hz, 2H), 3.12 (d, J=6.6 Hz, 2H), 2.45 (d, J=5.5 Hz, 2H), 2.01 (s, 1H), 1.66 (d, J=6.8 Hz, 2H), 0.98 (d, J=7.1 Hz, 3H). ¹³C NMR (200 MHz; CDCl₃): δ 168.6, 141.1, 138.6, 129.2, 127.7, 81.7, 70.2, 46.2, 30.3, 22.6, 19.4, 11.3. LRMS 344.1.

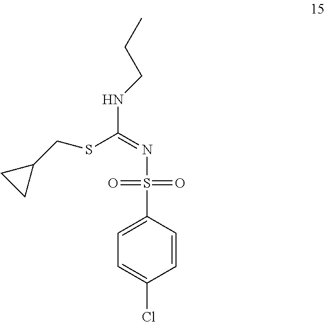 [SMILES: CCCN/C(=N/S(=O)(=O)c1ccc(Cl)cc1)SCC1CC1]

Cyclopropylmethyl N′-((4-chlorophenyl) sulfonyl)-N-propylcarbamimidothioate (15): Following General procedure E, Chlorpropamide (100 mg, 0.36 mmol) was converted in a three-step, one-pot protocol to title compound 24 (60 mg, 48% yield) as an off-white solid. ¹H NMR (800 MHz, CDCl₃): δ 8.18 (s, 1H), 7.83 (d, J=8.3 Hz, 2H), 7.44 (d, J=8.5 Hz, 2H), 3.26 (t, J=6.2 Hz, 2H), 2.93 (d, J=7.3 Hz, 2H), 1.66 (d, J=7.2 Hz, 2H), 0.98 (t, J=7.3 Hz, 4H), 0.55 (d, J=7.2 Hz, 2H). ¹³C NMR (200 MHZ; CDCl₃): δ 169.9, 141.5, 138.5, 129.1, 127.8, 46.2, 37.7, 29.8, 22.7, 11.3, 10.1, 6.0. LRMS 347.1, HRMS (C₁₄H₂₀ClN₂O₂S₂) [M+H]⁺ found m/z 347.0659, calcd 347.0655.

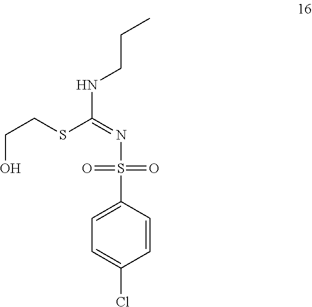 [SMILES: CCCN/C(=N/S(=O)(=O)c1ccc(Cl)cc1)SCCO]

2-Hydroxyethyl N′-((4-chlorophenyl) sulfonyl)-N-propylcarbamimidothioate (16): Following General procedure E, chlorpropamide (100 mg, 0.36 mmol) was converted in a three-step, one-pot protocol to yield title compound 25 (51 mg, 42% yield) as a sticky solid. ¹H NMR (800 MHZ, CDCl₃): δ 8.23 (s, 1H), 7.82 (d, J=4.4 Hz, 2H), 7.45 (d, J=4.3 Hz, 2H), 3.75 (d, J=5.2 Hz, 2H), 3.28 (d, J=4.9 Hz, 2H), 3.17 (d, J=4.6 Hz, 2H), 1.66 (t, J=5.9 Hz, 2H), 0.97 (d, J=4.9 Hz, 3H). ¹³C NMR (200 MHZ; CDCl₃): δ 169.5, 140.8, 138.8, 129.3, 127.7, 61.7, 46.3, 34.1, 22.6, 11.3. LRMS 337.1, HRMS (C₁₂H₁₈ClN₂O₃S₂) [M+H]⁺ found m/z 337.0443, calcd 337.0447.

### Example 7: Sulfonylselenylurea Formation and Alkylation

General Procedure F for Sulfonylselenylurea Formation and Alkylation: To a mixture of sulfonylurea compound (e,g 1a) (0.51 g, 1.00 mmol) in toluene (5 mL), POCl₃ (0.14 mL, 1.5 mmol), was added, followed by the addition of N,N-diisopropylethylamine (DIPEA) (0.26 mL, 1.5 mmol) and the mixture was heated to 95° C. for 1.5 h under N₂ atmosphere. The reaction mixture was then cooled, and the excess reagents in toluene were evaporated in vacuo. The imidoyl chloride intermediate was dissolved in dioxane (10 mL), to this solution was added dropwise, premade aq. Na₂SeSO₃ (2 eq) and the reaction was heated to 90° C. Upon consumption of chloro-intermediate (TLC/LCMS), alkylating agent was added dropwise to the reaction mixture reaction continued until all the “selenourea” was consumed. The reaction was cooled to room temperature and dioxane was evaporated. The organic mixture was the extracted into dichloromethane, washed with brine, and dried over Na₂SO₄. The solvent was removed in vacuo and dried thoroughly to afford seleno-compounds. The resultant solid was purified by flash chromatography (20% hexanes in EtOAc) to yield novel seleno-compounds as powders.

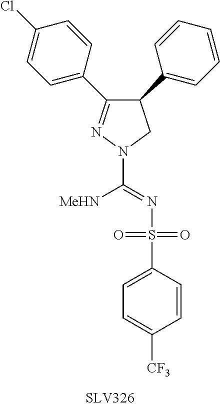 [SMILES: CN/C(=N\S(=O)(=O)c1ccc(C(F)(F)F)cc1)N1C[C@H](c2ccccc2)C(c2ccc(Cl)cc2)=N1]

SLV326 (S-enantiomer): To a solution of Compound 31 in DCM (5 mL) (20 mg, 0.03 mmol) cooled to 0° C., methylamine (1.5 eq) dissolved in DCM: MeOH (9:1) was added followed by Et₃N (1.5 eq). The reaction was warmed to room temperature and allowed to run until completion of the reaction (4 h). The organic layer was then extracted in DCM washed with water, dried over Na₂SO₄. Purification on a silica gel column using hexanes-ethyl acetate (1:1) yielded compound SLV326 as a pale white solid (14 mg, 84%). SLV326 was confirmed as the desired product with 97.9% ee by the comparison of all spectroscopic data with racemic sample. 1H NMR (800 MHZ, CDCl₃): δ 8.04 (d, J=7.8 Hz, 2H), 7.68 (d, J=8.1 Hz, 2H), 7.52 (d, J=8.5 Hz, 2H), 7.30 (d, J=7.5 Hz, 2H), 7.25 (s, 3H), 7.12 (d, J=7.1 Hz, 2H), 4.65 (d, J=1.1 Hz, 1H), 4.54-4.53 (m, 1H), 4.10 (s, 1H), 3.23 (s, 3H). LRMS 531.1, HRMS (C₂₄H₂₁ClN₄O₂F₃S₂) [M+H]⁺ found m/z 521.1031, calcd 531.1026. (R,R)-Whelk-O® 1 chiral column (250 mm×4.6 mm/5 μm, available from Regis Technologies, Inc., Morton Grove, IL): 100% EtOH (0.5 mL/min) (254 nm) Peak 1=11.1 min, Peak 2=NA (11.8 min). A racemic sample was used for ee calculations.

A generic scheme for this and related syntheses is presented below:

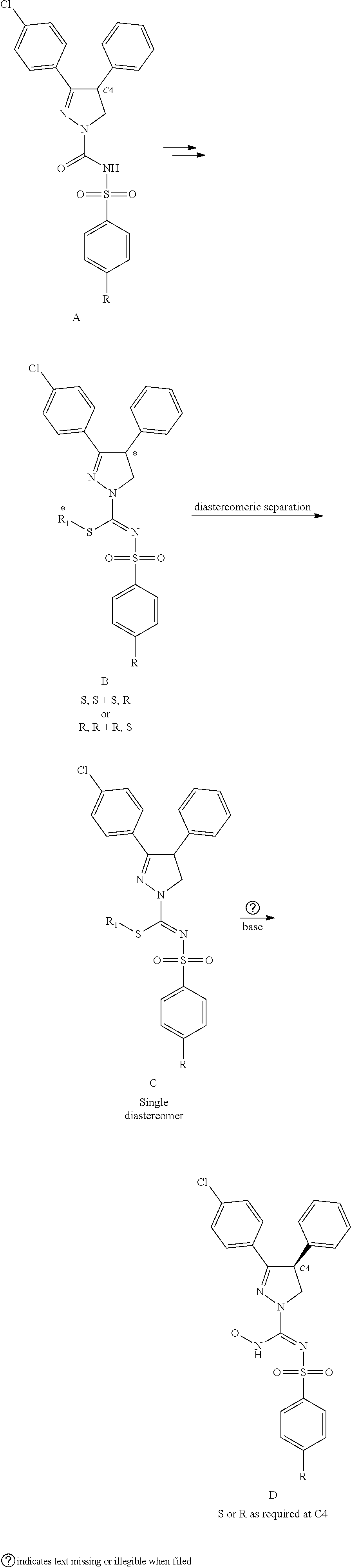

### Example 8: X-Ray Crystal Structures

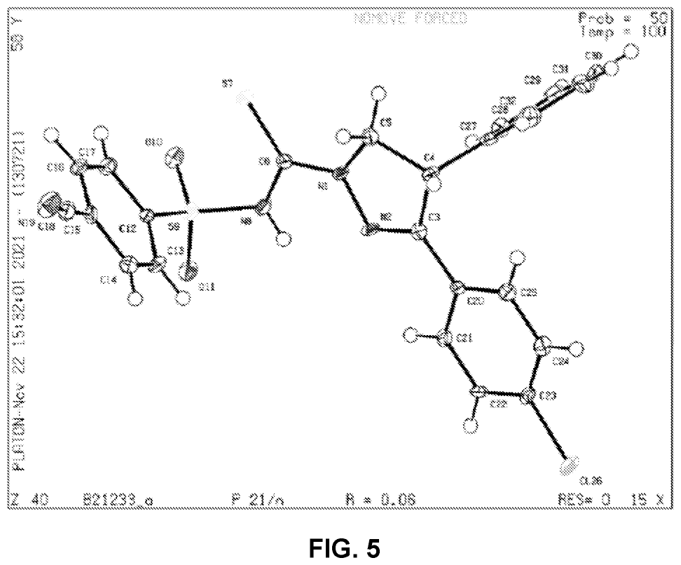

Compound 3m: A single crystal of 3m (CCDC 2158144) was mounted under mineral oil on a Mitegen micromount and immediately placed in a cold nitrogen stream at 100 (2) K prior to data collection. Data were collected on a Bruker D8 Quest equipped with a Photon100 CMOS detector and Mo ImS source. A series of 0.5° φ- and ω-scans were collected with monochromatic Mo Kα radiation, λ=0.71073 Å and integrated with the Bruker SAINT program. Structure solution and refinement was performed using the SHELXTL/PC suite and ShelXle. Intensities were corrected for Lorentz and polarization effects and an empirical absorption correction was applied using Blessing's method as incorporated into the program SADABS. Non-hydrogen atoms were refined with anisotropic thermal parameters. Further comments on each compound: 3m. The molecule crystallized in the Monoclinic P2₁/n space group. The amine H atom was located in the difference map and its position was allowed to freely refine. Remaining H atoms were included as riding idealized contributors. Amine H atom U's were assigned as 1.5 times U_{eq} of the carrier atom; remaining H atom U's were assigned as 1. 2 times carrier U_{eq}. See FIG. 5. Crystal data and structure refinement are presented in Table 5 below:

TABLE 5
Crystal Data and Structure Refinement for Compound 3m

| Identification code | MRI3208 | |
|---|---|---|
| Empirical formula | C23 H17 Cl N4 O2 S2 | |
| Formula weight | 480.98 | |
| Temperature | 100(2) | K |
| Wavelength | 0.71073 | Å |
| Crystal system | Monoclinic | |
| Space group | P2₁/n | |
| Unit cell dimensions | a = 5.7807(5) Å α = 90°. | |
| | b = 24.0953(18) Å β = 97.670(3)°. | |
| | c = 15.7069(12) Å γ = 90°. | |
| Volume | 2168.2(3) | Å3 |
| Z | 4 | |
| Density (calculated) | 1.473 | Mg/m³ |
| Absorption coefficient | 0.399 | mm⁻¹ |
| F(000) | 992 | |
| Crystal size | 0.622 × 0.056 × 0.018 | mm³ |

| | |
|---|---|
| Theta range for data collection | 2.138 to 25.437°. |
| Index ranges | −6 <= h <= 6, −29 <= |
| | k <= 29, −18 <= l <= 18 |
| Reflections collected | 50440 |
| Independent reflections | 3971 [R(int) = 0.2226] |
| Completeness to theta = 25.242° | 99.9% |
| Absorption correction | Semi-empirical from equivalents |
| Max. and min. transmission | 0.99558 and 0.87253 |
| Refinement method | Full-matrix least-squares on F² |
| Data/restraints/parameters | 3971/0/292 |
| Goodness-of-fit on F² | 1.062 |
| Final R indices [I > 2sigma(I)] | R1 = 0.0563, wR2 = 0.1083 |
| R indices (all data) | R1 = 0.0919, wR2 = 0.1204 |
| Extinction coefficient | n/a |
| Largest diff. peak and hole | 0.325 and −0.460 e · Å⁻³ |

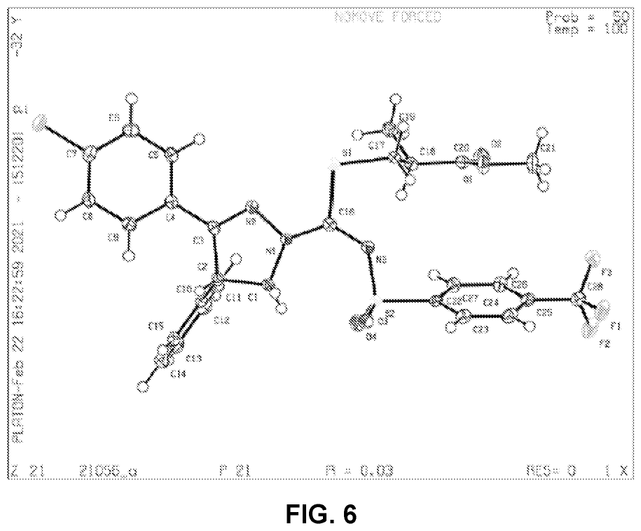

Compound 31: Single crystals of each compound, 31 (CCDC 2158143) were mounted under mineral oil on a Mitegen micromount and immediately placed in a cold nitrogen stream at 100 (2) K prior to data collection. Data were collected on a Bruker DUO equipped with an APEXII CCD detector and Cu ImS source. A series of 0.5° φ- and ω-scans were collected with monochromatic Cu Kα radiation, λ=1.54178 Å and integrated with the Bruker SAINT program. Structure solution and refinement was performed using the SHELXTL/PC suite and ShelXle. Intensities were corrected for Lorentz and polarization effects and an empirical absorption correction was applied using Blessing's method as incorporated into the program SADABS. Non-hydrogen atoms were refined with anisotropic thermal parameters. Further comments on each compound: 31. The molecule crystallized in the Monoclinic P2₁ space group. Methyl H atom positions, R—CH₃, were optimized by rotation about R—C bonds with idealized C—H, R—H and H—H distances. Remaining H atoms were included as riding idealized contributors. Methyl H atom U's were assigned as 1.5 times U_{eq} of the carrier atom; remaining H atom U's were assigned as 1.2 times carrier U_{eq} See FIG. 6. Crystal data and structure refinement are presented in Table 6 below:

TABLE 6
Crystal Data and Structure Refinement for Compound 3m

| Identification code | MRI2941E1 | |
|---|---|---|
| Empirical formula | C28 H25 Cl F3 N3 O4 S2 | |
| Formula weight | 624.08 | |
| Temperature | 100(2) | K |
| Wavelength | 1.54178 | Å |
| Crystal system | Monoclinic | |
| Space group | P2₁ | |
| Unit cell dimensions | a = 9.0363(3) Å α = 90°. | |
| | b = 8.4186(3) Å β = 92.351(2)°. | |
| | c = 18.2914(7) Å γ = 90°. | |
| Volume | 1390.31(9) | Å³ |
| Z | 2 | |
| Density (calculated) | 1.491 | Mg/m₃ |
| Absorption coefficient | 3.157 | mm⁻¹ |
| F(000) | 644 | |
| Crystal size | 0.209 × 0.114 × 0.030 | mm³ |

| | |
|---|---|
| Theta range for data collection | 2.417 to 68.337°. |
| Index ranges | −10 <= h <= 10, −9 <= |
| | k <= 9, −22 <= l <= 22 |
| Reflections collected | 28434 |
| Independent reflections | 4933 [R(int) = 0.0503] |
| Completeness to theta = 67.679° | 98.8% |
| Absorption correction | Semi-empirical from equivalents |
| Max. and min. transmission | 0.94315 and 0.68238 |
| Refinement method | Full-matrix least-squares on F² |
| Data/restraints/parameters | 4933/1/372 |
| Goodness-of-fit on F² | 1.054 |
| Final R indices [I > 2sigma(I)] | R1 = 0.0295, wR2 = 0.0770 |
| R indices (all data) | R1 = 0.0308, wR2 = 0.0780 |
| Absolute structure parameter | −0.006(9) |
| Extinction coefficient | n/a |
| Largest diff. peak and hole | 0.286 and −0.452 e · Å⁻³ |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Alex, J. M. et al. 4,5-Dihydro-1H-pyrazole: an indispensable scaffold. J Enzyme Inhib Med Chem 29, 427-442 (2014).
2. Al-Wahaibi, L. H., et al. Adamantane-Isothiourea Hybrid Derivatives: Synthesis, Characterization, In Vitro Antimicrobial, and In Vivo Hypoglycemic Activities. Molecules 22, (2017).
3. Amoroso, S.; et al. Glucose, Sulfonylureas, and Neurotransmitter Release: Role of ATP-Sensitive K+ Channels. Science 1990, 247, 852-854.
4. Appleby, A. P.; et al. Weed Control. In Ullmann's encyclopedia of industrial chemistry; Wiley-VCH Verlag Gmbh & Co. KGaA, Ed.; Wiley-VCH Verlag Gmbh & Co. KGaA: Weinheim, Germany, 2000.
5. Baruah, P. et al. Sulfonylurea Class of Antidiabetic Drugs Inhibit Acetylcholinesterase Activity: Unexplored Auxiliary Pharmacological Benefit toward Alzheimer's Disease. ACS Pharmacol. Transl. Sci. (2021).
6. Beno, B. R.; et al A Survey of the Role of Noncovalent Sulfur Interactions in Drug Design. J. Med. Chem. 2015, 58, 4383-4438.
7. Biswas, K.; et al. Bunte Salts and Congeners as Efficient Sulfur Surrogates: Recent Advances. COCAT 2019, 5, 182-195.
8. Brown, H. M. Mode of Action, Crop Selectivity, and Soil Relations of the Sulfonylurea Herbicides. Pestic. Sci. 1990, 29, 263-281.
9. Burch, J. D. et al. Naphthalene/quinoline amides and sulfonylureas as potent and selective antagonists of the EP4 receptor. Bioorg. Med. Chem. Lett. 21, 1041-1046 (2011).
10. Chorvat, R. J. Peripherally restricted CB1 receptor blockers. Bioorg. Med. Chem. Lett. 23, 4751-4760 (2013).
11. Cinar, R.; et al. Hybrid Inhibitor of Peripheral Cannabinoid-1 Receptors and Inducible Nitric Oxide Synthase Mitigates Liver Fibrosis. JCI Insight 2016, 1.
12. Cinar, R., et al. The therapeutic potential of second and third generation CB1R antagonists. Pharmacol. Ther. 208, 107477 (2020).
13. Coll, R. C.; et al. A Small-Molecule Inhibitor of the NLRP3 Inflammasome for the Treatment of Inflammatory Diseases. Nat. Med. 2015, 21, 248-255.
14. Crich, D., et al. Allylic selenosulfide rearrangement: a method for chemical ligation to cysteine and other thiols. J. Am. Chem. Soc. 128, 2544-2545 (2006).
15. De Ventura, T.; et al. Recent Advances in the Synthesis of Sulfonylureas. European J. Org. Chem. 2021, 2021, 1201-1214.
16. Ding, C. et al. One-step construction of unsymmetrical thioureas and oxazolidinethiones from amines and carbon disulfide via a cascade reaction sequence. RSC Adv. 9, 26768-26772 (2019).
17. Distler, H. The chemistry of bunte salts. Angew. Chem. Int. Ed. Engl. 6, 544-553 (1967).
18. Faidallah, H. M.; et al. Synthesis and Characterization of a Novel Series of Benzenesulfonylurea and Thiourea Derivatives of 2H-Pyran and 2H-Pyridine-2-Ones as Antibacterial, Antimycobacterial and Antifungal Agents. Eur. J. Chem. 2011, 2, 243-250.
19. Foye, W. O.; et al. A Direct Synthesis of Heterocyclic Thiols. Phosphorus and Sulfur and the Related Elements 1980, 8, 205-207.
20. Grosscurt, A. C. et al. 1-Phenylcarbamoyl-2-pyrazolines, a new class of insecticides. 3. Synthesis and insecticidal properties of 3,4-diphenyl-1-phenylcarbamoyl-2-pyrazolines. J. Agric. Food Chem. 27, 406-409 (1979).
21 Hay, J. V. Chemistry of Sulfonylurea Herbicides. Pestic. Sci. 1990, 29, 247-261.
22. Hermanson, G. T. in Bioconjugate Techniques 229-258 (Elsevier, 2013).
23. Iyer, M. R.; et al. Design, Synthesis, and Biological Evaluation of Novel, Non-Brain-Penetrant, Hybrid Cannabinoid CB1R Inverse Agonist/Inducible Nitric Oxide Synthase (INOS) Inhibitors for the Treatment of Liver Fibrosis. J. Med. Chem. 2017, 60, 1126-1141.
24. Iyer, M. R. et al. Synthesis, Biological Evaluation, and Molecular Modeling Studies of 3,4-Diarylpyrazoline Series of Compounds as Potent, Nonbrain Penetrant Antagonists of Cannabinoid-1 (CB1R)Receptor with Reduced Lipophilicity. J. Med. Chem. 65, 2374-2387 (2022).
25. Iyer, M. R., et al. Synthesis of 13 C6-labeled, dual-target inhibitor of cannabinoid-1 receptor (CB1 R) and inducible nitric oxide synthase (iNOS). J. Labelled Comp. Radiopharm. (2018). doi: 10.1002/jlcr.3639
26. Iyer, M. R., et al. Synthesis of S-2-((S)-3-(4-chlorophenyl)-N′-((4-chlorophenyl) sulfonyl)-4-phenyl-4,5-dihydro-1H-pyrazole-1-carboximidamido)-3-(methyl-d3) butanamide-d5, octadeuterated JD5037. J. Labelled Comp. Radiopharm. 60, 460-465 (2017).
27. Kulik, K.; et al. Different Oxidation Pathways of 2-Selenouracil and 2-Thiouracil, Natural Components of Transfer RNA. Int. J. Mol. Sci. 2020, 21.
28. Lange, J. H. M.; et al. A Novel Atom-Efficient, One-Pot Synthesis of Sulfonylguanidines and Sulfamoylguanidines. Tetrahedron Lett. 2011, 52, 3198-3200.
29 Lange, J. H. M. et al. Novel 3,4-diarylpyrazolines as potent cannabinoid CB1 receptor antagonists with lower lipophilicity. Bioorg. Med. Chem. Lett. 15, 4794-4798 (2005).
30. Lange, J. H. M., et al. Synthesis and SAR of 1,4,5,6-tetrahydropyridazines as potent cannabinoid CB1 receptor antagonists. Bioorg. Med. Chem. Lett. 19, 5675-5678 (2009).
31. Lange, J. H. M.; et al. Synthesis, Biological Properties, and Molecular Modeling Investigations of Novel 3,4-Diarylpyrazolines as Potent and Selective CB (1) Cannabinoid Receptor Antagonists. J. Med. Chem. 2004, 47, 627-643.
32. Liu, F. et al. A thiol-free synthesis of alkynyl chalcogenides by the copper-catalyzed C—X (X═S, Se) cross-coupling of alkynyl carboxylic acids with Bunte salts. Org. Chem. Front. (2018).
33. Liu, Z. et al. Functional Selectivity of a Biased Cannabinoid-1 Receptor (CB1R) Antagonist. ACS Pharmacol. Transl. Sci. 4, 1175-1187 (2021).
34. Loos, J. A.; et al. Anthelminthic Activity of Glibenclamide on Secondary Cystic Echinococcosis in Mice. PLOS Negl. Trop. Dis. 2017, 11, e0006111.
35 Mao, Z. et al. Anti-aging effects of chlorpropamide depend on mitochondrial complex-II and the production of mitochondrial reactive oxygen species. Acta Pharm. Sin. B 12, 665-677 (2022).
36. Masereel, B., et al. Anticonvulsant activity of pyrid-3-yl-sulfonyl ureas and thioureas. Epilepsia 38, 334-337 (1997).
37 Mishra, D. et al. A Unified Approach for the Synthesis of Isourea and Isothiourea from Isonitrile and N,N-Dibromoarylsulfonamides. J. Org. Chem. 86, 17581-17593 (2021).
38. Mustafa, M. et al. The importance of sulfur-containing motifs in drug design and discovery. Expert Opin. Drug Discov. 1-12 (2022).
39 Ohgami, N. et al. Glibenclamide acts as an inhibitor of acyl-CoA: cholesterol acyltransferase enzyme. Biochem. Biophys. Res. Commun. 277, 417-422 (2000).
40. Ozturk, T., et al. A Berzelius reagent, phosphorus decasulfide (P4S10), in organic syntheses. Chem. Rev. 110, 3419-3478 (2010).
41. Ozturk, T., et al. Use of Lawesson's reagent in organic syntheses. Chem. Rev. 107, 5210-5278 (2007).
42. Pala, G., et al. Hypoglycaemic activity of 1,1′-biadamantylaryl sulphonylureas. JPP 20, 559-563 (2011).
43. Pang, S. S., et al. Molecular basis of sulfonylurea herbicide inhibition of acetohydroxyacid synthase. J. Biol. Chem. 278, 7639-7644 (2003).
44. Parry, R. J. Biosynthesis of Some Sulfur-Containing Natural Products Investigations of the Mechanism of Carbon-Sulfur Bond Formation. Tetrahedron 1983, 39, 1215-1238.
45. Patlak, M. New Weapons to Combat an Ancient Disease: Treating Diabetes. FASEB J. 2002, 16, 1853.
46. Perlovich, G. L. et al. Novel isothiourea derivatives as potent neuroprotectors and cognition enhancers: synthesis, biological and physicochemical properties. J. Med. Chem. 52, 1845-1852 (2009).
47. Perregaux, D. G. et al. Identification and characterization of a novel class of interleukin-1 post-translational processing inhibitors. J. Pharmacol. Exp. Ther. 299, 187-197 (2001).
48. Proks, P.; et al. Sulfonylurea Stimulation of Insulin Secretion. Diabetes 2002, 51 Suppl 3, S368-76.
49 Ronchetti, R., et al. Recent advances in urea- and thiourea-containing compounds: focus on innovative approaches in medicinal chemistry and organic synthesis. RSC Med. Chem. 12, 1046-1064 (2021).
50 Sączewski, F.; et al. A New Imidazoline-Containing Bunte Salt: Synthesis, Molecular and Electronic Structure. Heterocycl. Comm. 2017, 23.
51. Sarmah, A. K.; et al. Hydrolysis of Sulfonylurea Herbicides in Soils and Aqueous Solutions: A Review. J. Agric. Food Chem. 2002, 50, 6253-6265.
52. Sharma, S. Thiophosgene in organic synthesis. Synthesis (Stuttg) 1978, 803-820 (1978).
53 Seino, S. Cell Signalling in Insulin Secretion: The Molecular Targets of ATP, CAMP and Sulfonylurea. Diabetologia 2012, 55, 2096-2108.
54. Sola, D.; et al. Sulfonylureas and Their Use in Clinical Practice. Arch. Med. Sci. 2015, 11, 840-848.
55. Solvay Pharmaceuticals B. V. 4,5-dihydro-1h-pyrazole derivatives having CB1-antagonistic activity. U.S. Pat. No. 6,476,060 (2002).
56 Southan, G. J., et al. Isothioureas: potent inhibitors of nitric oxide synthases with variable isoform selectivity. Br. J. Pharmacol. 114, 510-516 (1995).
57 Zhang, X., et al. Synthesis of chiral sulfinate esters by asymmetric condensation. Nature (2022).

1. A method for synthesizing a sulfonyl chalcogenide urea, the method comprising
(a) admixing a sulfonyl urea with a source of a leaving group to produce a first composition; and
(b) admixing the first composition with a compound having the formula M₂X₂O₃·mH₂O or M₂XJO₃·mH₂O to produce the sulfonyl chalcogenide urea,
wherein M is selected from K, Na, or Li;
wherein X and J are independently selected from S, Se, or Te; and wherein
m is from 0 to 6.

2. The method of claim 1, wherein the leaving group comprises a halide, wherein the halide is chloride, bromide, or iodide.

3. (canceled)

4. The method of claim 1 comprising:
(a) admixing a sulfonyl urea of Formula I with a source of a leaving group to form the first composition: 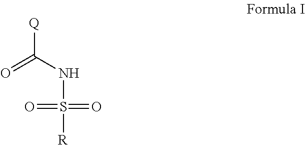
and
(b) admixing the first composition with the compound having the formula M₂X₂O₃·mH₂O or M₂XJO₃·mH₂O to form the sulfonyl chalcogenide urea having Formula II: 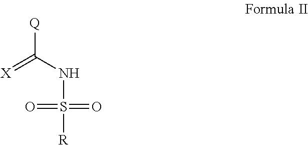
wherein X and J are independently selected from sulfur, selenium, or tellurium;
wherein Q is selected from a substituted or unsubstituted amine, substituted or unsubstituted heterocycloalkyl group, or a substituted or unsubstituted heteroaryl group, or any combination thereof; and
wherein R is selected from a substituted or unsubstituted amine, a substituted or unsubstituted alkyl group, cycloalkyl group, heterocycloalkyl group, or a substituted or unsubstituted aromatic group, or any combination thereof.

5. The method of claim 1, wherein the source of leaving group comprises a halogenating agent.

6. The method of claim 5, wherein the halogenating agent comprises a phosphorus halide, a dialkyl chloroiminium salt, a diaryl chloroiminium salt, an oxalyl halide, a carbonyl dihalide, or any combination thereof.

7. The method of claim 6, wherein the phosphorus halide has the formula PW₃, POW₃, or PW₅, wherein W is selected from Cl, Br, or I.

8. The method of claim 1, wherein step (a) further comprises admixing a base with the sulfonyl urea and the source of the leaving group.

9. The method of claim 8, wherein the base comprises N,N-diisopropylethylamine (DIPEA), triethylamine, 4-dimethylaminopyridine (DMAP), imidazole, benzimidazole, guanidine, ammonium hydroxide, pyridine, lutidine, sodium hydroxide, potassium hydroxide, 1,8-diazabicyclo[5.4.0]undec-7-ene (DBU), or any combination thereof.

10.-11. (canceled)

12. The method of claim 1, wherein the compound having the formula M₂X₂O₃·mH₂O or M₂XJO₃·mH₂O comprises Na₂S₂O₃19 5H₂O, Na₂S₂O₃, K₂S₂O₃, K₂Se₂O₃ or Na₂SeSO₃.

13. The method of claim 12, wherein X is sulfur and the compound having the formula M₂X₂O₃·mH₂O comprises Na₂S₂O₃ or K₂S₂O₃ or wherein X is selenium and the compound having the formula M₂X₂O₃·mH₂O or M₂XJO₃·mH₂O comprises K₂Se₂O₃ or Na₂SeSO₃.

14. (canceled)

15. The method of claim 1, wherein step (b) further comprises admixing an additive with the first composition and the compound having the formula M₂X₂O₃·mH₂O or M₂XJO₃·mH₂O, wherein the additive comprises triethylamine, Li₂CO₃, Na₂CO₃, K₂CO₃, MgCO₃, CaCO₃, BaCO₃, LiHCO₃, NaHCO₃, KHCO₃, Mg(HCO₃)₂, Ca(HCO₃)₂, tetrabutylammonium bromide, tetramethylammonium chloride, or any combination thereof.

16.-19. (canceled)

20. The method of claim 1, wherein Q is selected from:

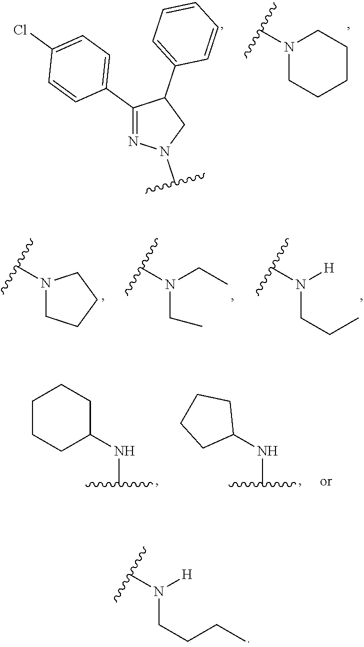

21. The method of claim 1, wherein R is selected from: 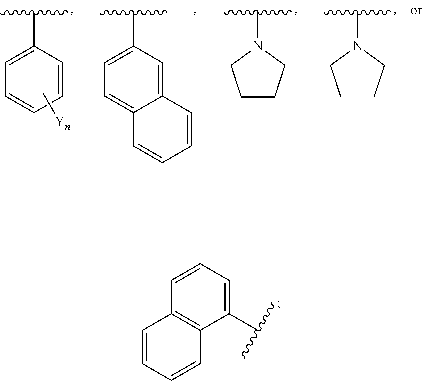
wherein each Y is independently selected from hydrogen, substituted or unsubstituted linear or branched C₁-C₁₀ alkyl, nitro, substituted or unsubstituted amino, halogen, cyano, alkoxy, thiol, phosphonate, haloalkyl, or haloalkoxy;
and wherein n is an integer from 1 to 5.

22. The method of claim 1, wherein prior to step (a), the sulfonyl urea is produced in situ by reacting a sulfonyl carbamate or an isocyanate with a substituted or unsubstituted amine.

23. The method of claim 22, wherein the sulfonyl carbamate is a compound of Formula III or Formula IIIa and the substituted or unsubstituted amine is a compound having formula Q-H: 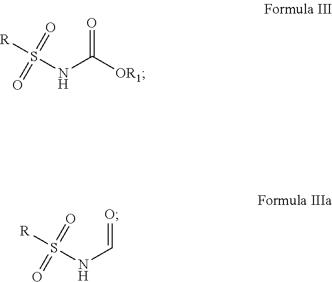
wherein R₁, when present, comprises a substituted or unsubstituted linear or branched alkyl, aryl, or aralkyl group; and
wherein R is selected from a substituted or unsubstituted amine, a substituted or unsubstituted alkyl group, cycloalkyl group, heterocycloalkyl group, or a substituted or unsubstituted aromatic group, or any combination thereof.

24. The method of claim 23, wherein R₁ is methyl, tert-butyl, or phenyl.

25. (canceled)

26. A sulfonyl chalcogenide urea having Formula II: 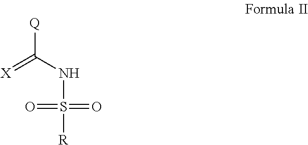
wherein X is sulfur, selenium, or tellurium;
wherein Q is selected from a substituted or unsubstituted amine, substituted or unsubstituted heterocycloalkyl group, or a substituted or unsubstituted heteroaryl group, or any combination thereof;
wherein R is selected from a substituted or unsubstituted amine, a substituted or unsubstituted alkyl group, cycloalkyl group, heterocycloalkyl group, or a substituted or unsubstituted aromatic group, or any combination thereof; and
wherein, when the compound has one or more stereocenters, the compound of Formula II can be present as an (R) enantiomer at the one or more stereocenters, an (S) enantiomer at the one or more stereocenters, a mixture of (R) and (S) enantiomers at the one or more stereocenters, or any combination thereof.

27. The sulfonyl chalcogenide urea of claim 26, wherein Q is selected from:

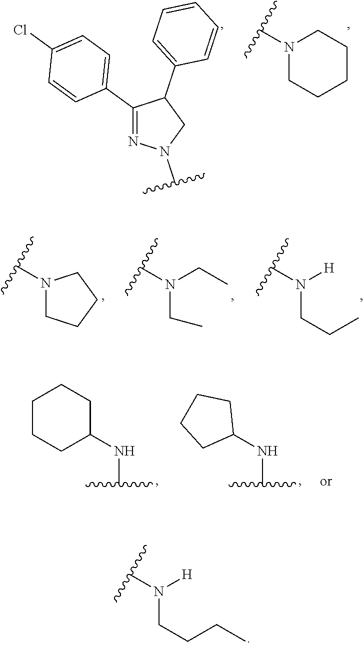

28. The sulfonyl chalcogenide urea of claim 26 or 27, wherein R is selected from: 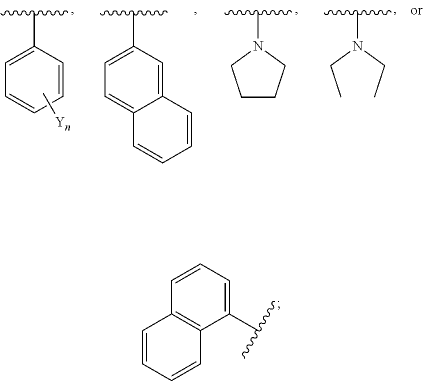
wherein each Y is independently selected from hydrogen, substituted or unsubstituted linear or branched C₁-C₁₀ alkyl, nitro, substituted or unsubstituted amino, halogen, cyano, alkoxy, thiol, phosphonate, haloalkyl, or haloalkoxy; and
wherein n is an integer from 1 to 5.

29. The sulfonyl chalcogenide urea of claim 26, wherein the compound of Formula II is selected from: 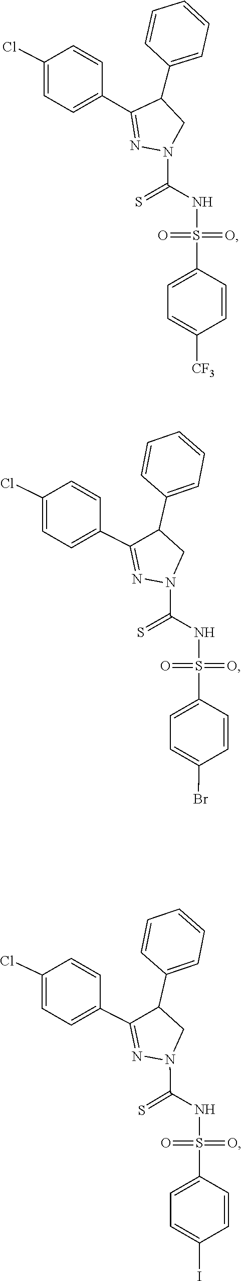 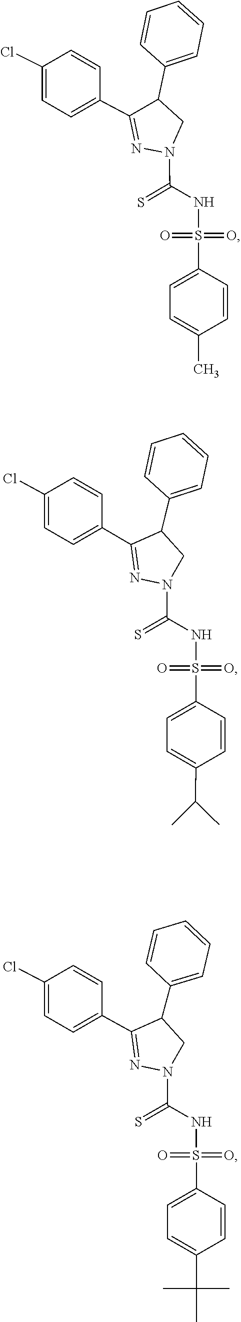 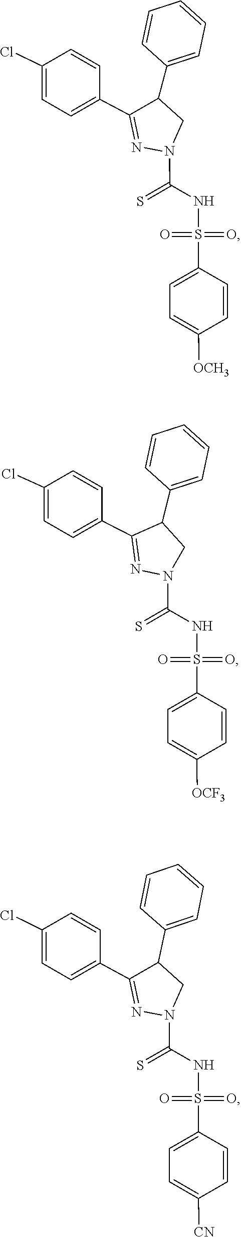 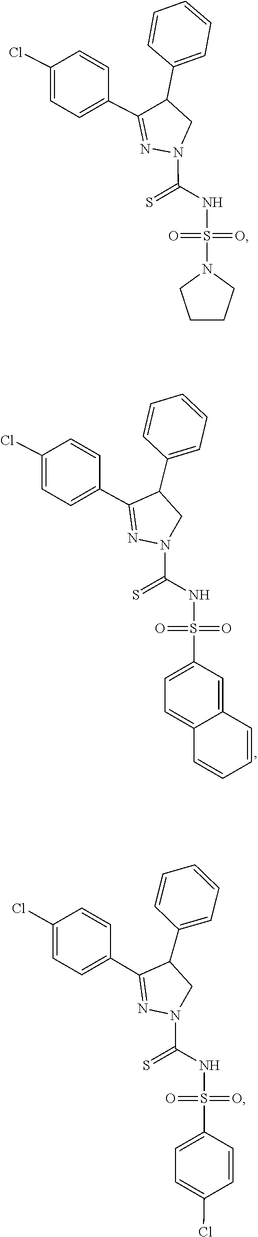 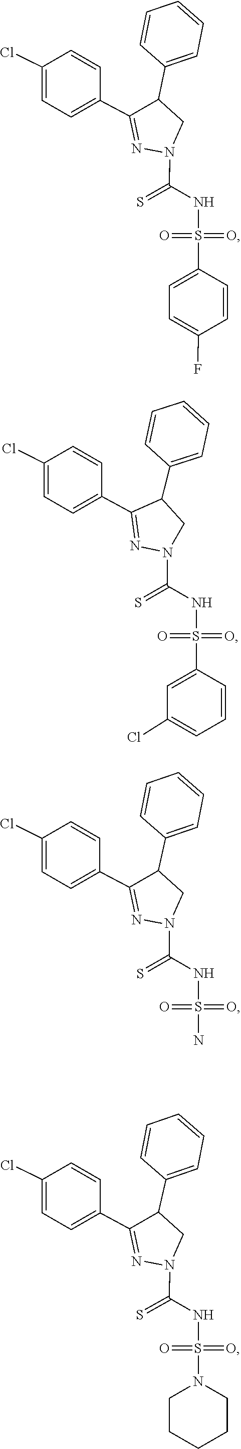 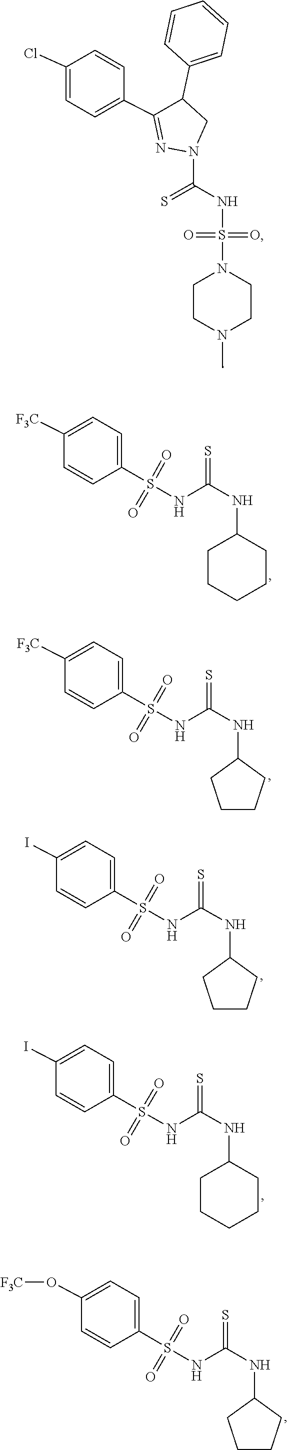 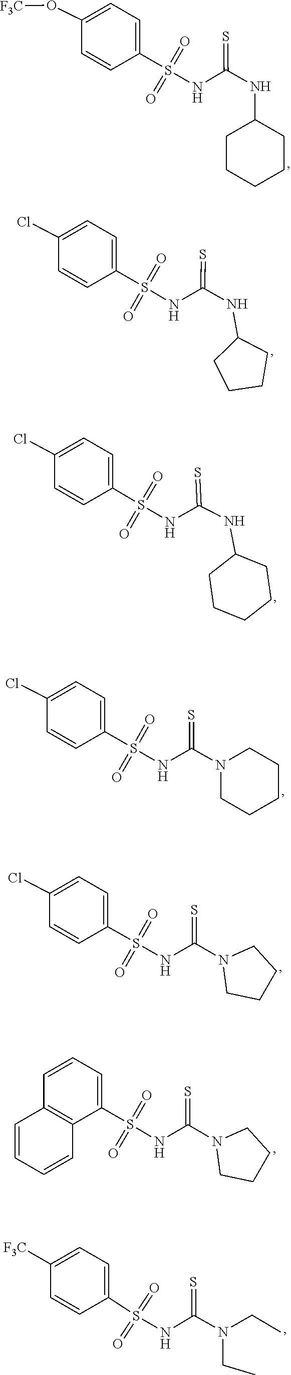 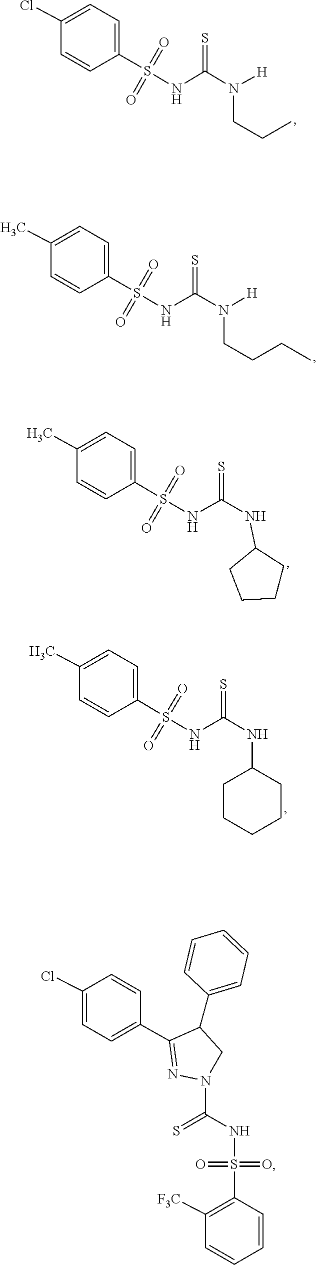 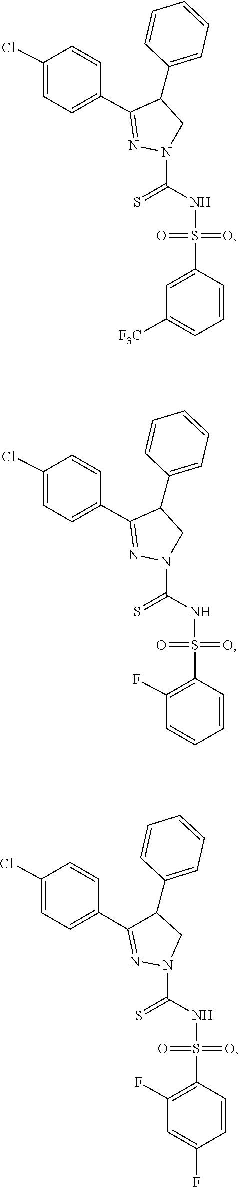 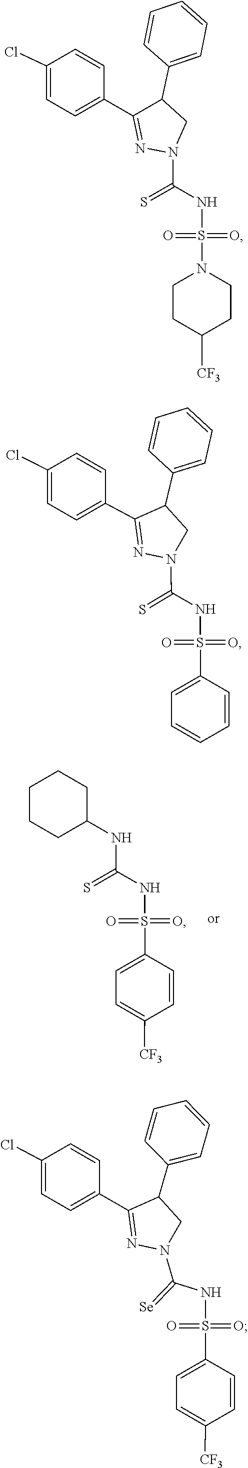
wherein, when the compound has one or more stereocenters, the compound of Formula II can be present as an (R) enantiomer at the one or more stereocenters, an (S) enantiomer at the one or more stereocenters, a mixture of (R) and (S) enantiomers at the one or more stereocenters, or any combination thereof.

30.-39. (canceled)
